(19) United States
(12) Patent Application Publication
(10) Pub. No.: US 2026/0130942 A1
(43) Pub. Date: May 14, 2026

(54) A METHOD OF PRODUCING EXTRACELLULAR VESICLES

(71) Applicant: AMNIOTICS AB, Lund (SE)

(72) Inventor: Jan Talts, Staffanstorp (SE)

(21) Appl. No.: 18/861,030

(22) PCT Filed: Apr. 28, 2023
(86) PCT No.: PCT/EP2023/061254

§ 371 Date: Oct. 28, 2024

(30) Foreign Application Priority Data
Apr. 29, 2022 (SE) 2250521-8

(51) Int. Cl. A61K 35/28; A61K 45/06; A61P 17/02; C12N 5/00; C12N 5/0775

(52) U.S. Cl. CPC A61K 35/28; A61K 45/06; A61P 17/02; C12N 5/0081; C12N 5/0668

(57) ABSTRACT

The present invention relates to extracellular vesicles derived from mesenchymal stem cells and their use in medicine.

### FIELD OF INVENTION

The present invention relates to the field of extracellular vesicles derived from stem cells and their use in medicine.

### BACKGROUND

Cells secrete extracellular vesicles (EVs) that may have an endosomal origin, or they are derived from evaginations of the plasma membrane. The former type is called exosomes and range in size from 50-100 nm. These EVs contain molecules such as nucleic acids and other proteins. The composition of the EVs depends on the cell type and its physiological conditions. These factors modulate various metabolic and signalling pathways. Thereby, EVs can be applied as diagnostic and therapeutic tools in medicine and preferentially within skin wound healing therapy.

Mesenchymal stem cells (MSCs) can be found in nearly all tissues and are mostly located in perivascular niches. As will be understood by one of skill in the art, MSCs are multipotent stromal cells capable of differentiating into numerous cell types, and possess anti-inflammatory, angiogenic properties for directing tissue repair processes, thereby making MSCs valuable for therapeutic treatments. Term amniotic fluid (TAF) collected during a caesarean section contains several valuable cells, including MSCs. Moreover, specific subpopulations of MSCs are likely to be particularly well suited to use for production of therapeutic drugs. Previously, MSCs sourced from adult bone marrow, adult adipose tissue or neonatal birth-associated tissues including placenta, umbilical cord and cord blood were extensively used to obtain MSCs. MSCs from these neonatal tissues may have additional capacities in comparison to MSCs derived from adult sources. Indeed, several studies have reported superior biological properties such as improved proliferative capacity, life span and differentiation potential of MSCs from birth-associated tissues over adult derived MSCs.

MSCs have immunomodulatory properties and a high regenerative capacity. It has been shown that EVs derived from MSCs have immunosuppressive and immunomodulatory properties. In addition, they can activate angiogenesis, proliferation, migration and differentiation of the main cell types involved in skin regeneration, such as endothelial cells, fibroblasts and keratinocytes.

EVs are lipid-bilayer spheroid structures, without replicating capacity, that are released from cells. These structures are preserved by evolution and have a function in intercellular communication. The EVs are traditionally classified into four subtypes (Table A).

TABLE A
Traditional classification of extracellular vesicles.

| Extracellular vesicle type | Size |
|---|---|
| Endosomal exosomes | 50-100 nm |
| Microvesicles from the plasma membrane | 20-1000 nm |
| Membrane particles | 50-600 nm |
| Apoptotic vesicles | 1000-5000 nm |

MSCs from different sources have been used in wound healing. Several studies have demonstrated that conditioned media from MSC cultures have a similar—or even higher—regenerative capacity than the MSCs themselves, when applied to wounds. Thus, the regenerative capacity of MSCs could be due to their paracrine activity.

The skin is the largest organ, accounting for 16% of the body weight. It is made up of three main layers: epidermis, dermis and hypodermis. The epidermis has five sublayers, which from the outside is: stratum corneum, stratum lucidum, stratum granulosum, stratum spinosum and stratum basale. The epidermis is made up of keratinocytes to 95%. The keratinocytes are proliferative in the stratum basale and differentiating and replacing the ones of the other sublayers.

There are four overlapping stages during skin wound healing: haemostasis, inflammation, proliferation and maturation/remodelling. It has been shown that EV derived from bone marrow MSC increases migration and proliferation of dermal fibroblasts and angiogenesis in human umbilical-vein endothelial cells (HUVEC) (Casada-Díaz et al., 2020).

One problem associated with applying e.g. bone marrow derived MSC's is to obtain a sufficient amount of starting material for commercial manufacture. Therefore, it is very difficult to get bone marrow derived cells or EVs from these cells to the market.

Based on the above there is a need to solve the problems in the art and progress the commercial use of extracellular vesicles (EVs) from MSCs.

The listing or discussion of an apparently prior-published document in this specification should not necessarily be taken as an acknowledgement that the document is part of the state of the art or is common general knowledge.

### SUMMARY OF INVENTION

Accordingly, it is an object of the present invention to advance the assessment of extracellular vesicles (EVs) from MSCs and their uses in therapy. It is a further object of the present invention to identify alternative and improved MSC sources of EVs.

The type of MSCs used herein to obtain EVs are derived from term amniotic fluid (TAF MSCs), which have been further characterised by panels of markers to identify organ-specific subsets of TAF MSCs (such as skin TAF MSCs). EVs derived from TAF MSCs have not previously been studied. TAF collected during a cesarean section contains several valuable cells, including MSCs. Amniotic fluid is today considered medical waste that is discarded. Therefore, both the ethical and practical incentive to harvest such an untapped resource is clear.

Certain disclosed examples relate to devices, cells, methods, uses, and systems for amniotic mesenchymal stem cells from amniotic fluid and cells derived thereof. It will be understood by one of skill in the art that application of the devices, methods, uses, and systems described herein are not limited to a particular cell or tissue type. Further examples are described below.

A first aspect of the invention relates to a method for obtaining extracellular vesicles from term amniotic fluid mesenchymal stem cells (TAF MSCs), comprising: providing term amniotic fluid (TAF); removing particulate material from the TAF to obtain purified TAF cells; performing adherence selection on the purified TAF cells to obtain TAF adherence cells; passaging the TAF adherence cells to obtain a population of cells comprising TAF MSCs; propagating the TAF MSCs; and obtaining extracellular vesicles from the TAF MSCs.

A second aspect of the invention relates to isolated extracellular vesicles, wherein: (a) the isolated extracellular vesicles are produced by the method according to the first aspect of the invention; (b) the purity is a ratio based on an ExoView/ZetaView ratio based on CD63, CD81 and/or CD9; and/or (c) the isolated extracellular vesicles express at least one surface marker associated with skin TAF MSCs, lung TAF MSCs, kidney TAF MSCs, or neural TAF MSCs.

A third aspect of the invention relates to a decellularised composition comprising the isolated extracellular vesicles according to the second aspect of the invention.

A fourth aspect of the invention relates to the isolated extracellular vesicles according to the second aspect of the invention, or the decellularised composition according to the third aspect of the invention, for use in medicine.

A fifth aspect of the invention relates to isolated extracellular vesicles according to the second aspect of the invention, or the decellularised composition according to the third aspect of the invention, for use in preventing and/or treating: (a) a skin condition (such as chronic skin ulcers, also referred to as chronic cutaneous ulcers, diabetic skin ulcers, bedsores); (b) fibrotic diseases (such as scar formation); (c) cancer or tumours, multiple sclerosis, amyotrophic lateral sclerosis, cardiovascular diseases (e.g. stroke, acute and chronic heart failure, atherosclerosis), diabetes, arthritis (e.g. rheumatoid arthritis and osteoarthritis), osteonecrosis, lumbar intervertebral disc degeneration, bowel disease (e.g. Crohn's disease), kidney and liver chronic disease, sepsis, spinal cord contusions, critical limb ischemia, neurodegenerative diseases, atherosclerosis; (d) skin, renal, liver, and neural injuries; (e) an adverse immune response, such as inflammation (e.g. by modulating NFkB signalling); and/or (f) transplantation/GVHD.

A sixth aspect of the invention relates to the isolated extracellular vesicles according to the second aspect of the invention, or the decellularised composition according to the third aspect of the invention, for use in wound healing.

A seventh aspect of the invention relates to a non-therapeutic use of the isolated extracellular vesicles according to the second aspect of the invention, or the decellularised composition according to the third aspect of the invention, in antiaging.

An eighth aspect of the invention relates to a device comprising and/or embedded with the isolated extracellular vesicles according to the second aspect of the invention, or the decellularised composition according to the third aspect of the invention.

### DESCRIPTION OF THE FIGURES

FIG. 1: A flow diagram showing the steps in the purification, culturing and selection of MSC subpopulations.

FIG. 2: Control images of TAF MSC confluence for the 24 hour (A), 48 hour (B) and 72 hour (C) flasks. Images were obtained at time zero (TO) for each flask at 4× magnification.

FIG. 3: Confluence following incubation of TAF MSCs for 24 hours (A), 48 hours (B) and 72 hours (C). Images were obtained at the respective times at 4× magnification.

FIG. 4: (A) Human TAF MSC were collected before planned Caesarean sections. Term Amniotic Fluid (TAF) collection. (B) Colony formation day 13 post seeding. (C) Passage 2. (D) Accumulative cell growth of TAF-MSCs.

FIG. 5: Photographic fimage of MACS Quant Tyto cell sorting apparatus.

FIG. 6: Schematic image of MACS Quant Tyto cell sorting apparatus.

FIG. 7: Flow cytometry analysis of unstained (A), unsorted (B), sorted positive (C) and sorted negative (D) for TAF-skin marker. Doublets and dead cells are excluded.

FIG. 8: Unsorted, sorted positive and negative sorted one passage after sorting shown by immunocytochemistry. DAPI stains the nuclei (ImaGene-IT) (Scale bars: 20 μm).

FIGS. 9: (A) and (B) MSC positive and negative markers confirmed the identity of the cells as MSCs. Flow cytometry analysis of MSC markers of skin TAF MSCs. Doublets and dead cells are excluded.

FIG. 10: Flow cytometry analysis of MSC markers of fetal skin MSCs (positive control for FIG. 9).

FIG. 11: Schematic of ZetaView (Particle Metrix™) with the Nanoparticle Tracking Analysis (NTA) technology.

FIG. 12: Schematic of ExoView analysis method summary.

FIG. 13: Tetraspanin analysis by ExoView: spot montage at 24 hours.

FIG. 14: Tetraspanin analysis by ExoView: spot montage at 48 hours.

FIG. 14: Tetraspanin analysis by ExoView: spot montage at 72 hours.

FIG. 16: Examples of disabled spots in CD81 at 48 hours (top panel) and CD9 at 72 hours (bottom panel).

FIG. 17: (A) Particle counts—24 h—All; numbers above the top of each quartet of bars are 3/3, 3/3, 3/3 and 3/3 spots; Y-axis=0, 2000, 4000, 6000, 8000, 10,000 and 12,000. (B) Particle counts—48 h—All; numbers above the top of each quartet of bars are 3/3, 2/3, 2/3 and 3/3 spots; Y-axis (×10⁴)=0, 0.2, 0.4, 0.6, 0.8, 1, 1.2, 1.4, 1.6, 1.8 and 2. (C) Particle counts 72 h-All; numbers above the top of each quartet of bars are 3/3, 3/3, 2/3 and 3/3 spots; Y-axis=0, 2000, 4000, 6000, 8000, 10,000 and 12,000. In all graphs (A-C), the quartets of bars, from left to right, correspond to Total, CD63, CD81 and CD9, respectively; and the X-axis categorises each quartet of bars, from left to right, as CD63, CD81, CD9 and MIgG, respectively.

FIG. 18: Colocalization analysis—24 h—All; CD63 total=8342, spots 3/3; CD81 total=10330, spots 3/3; CD9 total=7186, spots 3/3; MIgG total=52, spots 3/3. Colocalization analysis—48 h—All; CD63 total=8631, spots 3/3; CD81 total=11516, spots 2/3; CD9 total=8355, spots 2/3; MIgG total=285, spots 3/3. Colocalization analysis—72 h—All; CD63 total=7820, spots 3/3; CD81 total=9911, spots 3/3; CD9 total=7527, spots 2/3; MIgG total=244, spots 3/3. Channels: R (red)=CD63; G (green)=CD81; B (blue)=CD9; IM=Mouse IgG negative control.

FIG. 19: Schematic of the MACSPlex protocol and Bradford Assay. 1. Capture beads coupled to 37 exosomal epitope antibodies (CD3, CD4, CD19, CD8, HLA-DRDPDQ, CD56, CD105, CD2, CD1c, CD25, CD49e, ROR1, CD209, CD9, SSEA-4, HLA-ABC, CD63, CD40, CD62P, CD11c, CD81, MCSP, CD146, CD41b, CD42a, CD24, CD86, CD44, CD326, CD133/1, CD29, CD69, CD142, CD45, CD31, CD20, CD14). 2. EVs bind to specific antibodies on Capture Beads. 3. EVs bound to specific Capture Beads are labelled with APC-conjugated anti-EVs antibodies (CD9, CD63, CD81). Circles with schematic antibodies on the surface correspond to “Beads”. Circles to which schematic antibodies bind correspond to EVs. The bottom right of the schematic shows a graph with PE-A on the y-axis and FITC-A on the x-axis, with representative cells skewing in a positive correlation, wherein exemplary cells are labelled as CD49e, CD63, CD29, CD81 and CD9 for the Beads detection.

FIG. 20: Schematic of extracellular vesicles (EV) released from a cell.

FIG. 21: Left graph=Mean size of particles from EV preparation of medium selected at 0 h, 24 h, 48 h and 72 h of starvation. Right graph=Concentration of particles in the different time points.

FIG. 22: Skin TAF MSC-EVs (72 h) decrease inflammation as assessed by NFκB activation relative to LPS activation in a THP1-dual immunomodulation assay. (WS=whole secretome, UC-EV ultracentrifuged EVs). The y-axis represents “Normalised fluorescence”.

FIG. 23: Expression level of EV markers of 72 h EVs assessed by MACSPlex, a multiplexed marker analysis by flow cytometry. Dotted line represents significant expression level. (WS=whole secretome, UC-EV ultracentrifuged EVs) (EVerZom). “Exosome” indicates exosome markers. “MSC” indicates MSC and endothelial markers. “Blood” indicates platelets and Mk markers

FIG. 24: ZetaView analysis. (A) to flask 24 h measure 1; (B) to flask 24 h measure 2. GRUBBS_NUMBER=Deviation of the average number of particles is too large compared to other positions. Heterogeneous distribution of particles throughout the cell. MIN_TRACES=A particle must be followed for at least 5 or more traces. This message typically comes up when only few particles are in the sample. Due to Too few particles; concentration too low.

FIG. 25: ZetaView analysis. (A) to flask 48 h measure 1; (B) to flask 48 h measure 2. GRUBBS_NUMBER=Deviation of the average number of particles is too large compared to other positions. Heterogeneous distribution of particles throughout the cell. GRUBBS_SIZE=Deviation of the size of the particles is too large compared to other positions. Heterogeneous distribution of large particles throughout the cell.

FIG. 26: ZetaView analysis. (A) to flask 72 h measure 1; (B) to flask 72 h measure 2. GRUBBS_NUMBER=Deviation of the average number of particles is too large compared to other positions. Heterogeneous distribution of particles throughout the cell. Range_Num=The average number of particles is lower than 10 or larger than 800. Too few or too many particles.

FIG. 27: ZetaView analysis. (A) 24 h measure 1; (B) 24 h measure 2; (C) 24 h measure 3. GRUBBS_NUMBER=Deviation of the average number of particles is too large compared to other positions. Heterogeneous distribution of particles throughout the cell. GRUBBS_SIZE=Deviation of the size of the particles is too large compared to other positions. Heterogeneous distribution of large particles throughout the cell. GRUBBS_MI=Deviation of the mean intensity of the particles is too large compared to other positions. Causes: Air bubble, large particles, High scattering contaminations in the sample, Background from free fluorescence dye or protein monomers, Optics too close to one cell wall (in case of misalignment).

FIG. 28: ZetaView analysis. (A) 48 h measure 1; (B) 48 h measure 2. GRUBBS_NUMBER=Deviation of the average number of particles is too large compared to other positions. Heterogeneous distribution of particles throughout the cell.

FIG. 29: ZetaView analysis. (A) 72 h measure 1; (B) 72 h measure 2.

### DETAILED DESCRIPTION OF THE INVENTION

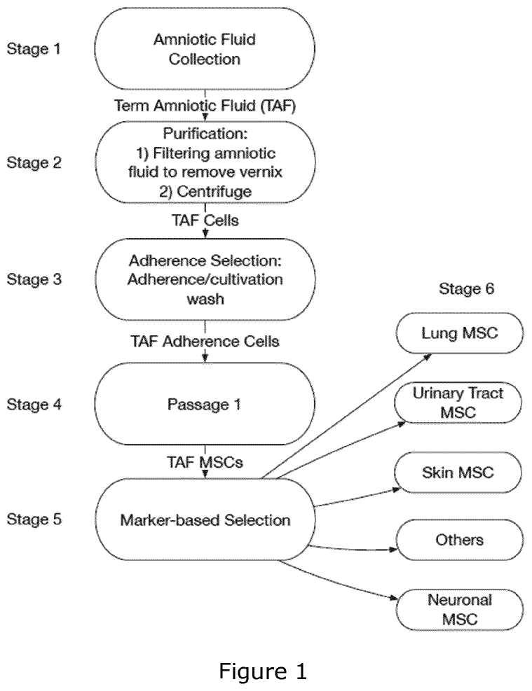

Methods of purifying, culturing and selecting MSC subpopulations with neonatal quality and adult tissue specificity are summarized in FIG. 1 and described in detail below. Examples disclosed herein relate to methods for collecting, purifying, isolating, expanding, differentiating, and maturing amniotic fluid-derived cells. The examples disclosed herein are not limited to collection of a certain type of amniotic-derived cell and the technologies disclosed herein are broadly applicable to different cells and tissues. Amniotic fluid may be collected to produce term amniotic fluid (TAF) and processed according to the methods described in U.S. patent application Ser. No. 14/776,499 (corresponding to US2016/0030489), the entire content of which is incorporated by reference.

The provision of TAF, removal of particulate material from the TAF to obtain purified TAF cells, adherence selection, and passaging of the TAF adherence cells may be in accordance with WO 2021/076042 A1 and/or WO 2021/076043 A1, the entire contents of which are incorporated by reference.

### Purification

Term amniotic fluid (TAF) is purified by filtering term amniotic fluid to remove vernix. Although the term ‘term amniotic fluid’ is employed here and elsewhere in the present disclosure, it is understood that methods, processes, and devices of the present disclosure may be applied to all amniotic fluids and not just term amniotic fluid. Term amniotic fluid may be amniotic fluid collected at term caesarean section deliveries using, for example, a closed catheter-based system. For the purposes of the present description, ‘term amniotic fluid’ may be amniotic fluid collected at planned cesarean sections after 37 completed weeks of pregnancy or later, or at planned cesarean section close to term, for example after 36 completed weeks of pregnancy. Preferably, term amniotic fluid is taken at planned caesarean sections during week 37 of pregnancy or later.

The amniotic fluid contains amniotic cells originating from the fetus or the amniotic sac such as mesenchymal stem cells (MSCs). The amniotic fluid also contains other materials chafed off the skin such as hair and vernix. Material other than the amniotic cells are here referred to as particulate matter and may also comprise meconium, blood clots, etc. Particulate matter may be considered as anything larger than 20 μm. For the purposes of filtering, it may be particularly advantageous to treat anything larger than 30 μm or even 50 μm as particulate matter. Optionally, anything larger than the targeted amniotic cells may be treated as particulate matter. The amniotic fluid thus generally contains a mixture of amniotic cells and particulate matter.

Removing particulate material from the TAF to obtain purified TAF cells may be done by applying any known method in the art such as filtration, centrifugation, etc. The TAF may be filtered through a filter having a pore size at or above 20 μm. The filter may be made from any synthetic material including but not limited to cellulose acetate, cellulose nitrate (collodion), polyamide (nylon), polycarbonate, polypropylene and polytetrafluoroethylene (Teflon).

### Adherence Selection

Various terms known to one skilled in the art have been and will be used throughout the specification, for example, the terms “express, expression, and/or expressing” in the context of a cell surface marker are meant to indicate the presence of a particular marker on the surface of a cell, said surface marker having been produced by the cell. Surface marker expression may be used to select between different cell populations, for example, positively selecting for surface marker expression indicates the selection of a cell population that more strongly expresses a particular surface marker as compared to another cell population. Conversely, negatively selecting for cell surface marker expression indicates the selection of a cell population that more weakly expresses a particular surface marker as compared to another cell population.

As explained above and elsewhere in the specifications, TAF contains various progenitor cell types. In certain examples, particular progenitor cell types may be isolated and propagated via adherence selection. For example, a vitronectin substrate, Synthemax (Merck, CORNING®, Synthemax®, II-SC SUBSTRATE, CLS3535-1EA) may be used as a coating to create a more in vivo-like environment for stem cell culture, thereby limiting maturation of the TAF-derived progenitor cells and maintaining plasticity. Synthemax is an animal-component free, synthetic, flexible vitronectin-based peptide substrate for serum or serum-free expansion of human progenitor/stem cells and other adult stem cell types. One of skill in the art will understand that the vitronectin-based peptide substrate may include a portion of a vitronectin protein, such as a particular peptide sequence of vitronectin. Alternatively, intact vitronectin protein may be used. Synthemax vitronectin substrate offers a synthetic, xeno-free alternative to biological coatings and/or feeder cell layers commonly used in cell culture and known in the art. Briefly, standard tissue-culture treated flasks may be coated with about 0.2 mL Synthemax/cm² at 10 μg/mL giving a surface density of 2 μg/cm², and incubated at 37° C. for about 1 h, 1.5 h, 2 h, 4 h, 8 h, or more than 8 h or at room temperature for about 2 h, 1 h, 4 h, 8 h or more than 8 h with surplus solution optionally being removed and replaced. In certain examples, Synthemax may be coated at a surface density of about: 1 to 5 μg/cm², such as 2 μg/cm², 1 to 10 μg/cm², 1.5 to 4 μg/cm², 1 to 3 μg/cm², or about 1.5 to 2.5 μg/cm².

In other embodiments, adherence selection can be performed using a surface coated with, for example, Collagen, Fibronectin. Alternatively, adherence selection can be performed using an uncoated surface comprising a tissue-culture treated plastic.

Cells purified from TAF fluid may be gently re-suspended in prewarmed xeno-free cell culture media, with the cell suspension is then added to the Synthemax-coated flasks. Media may be changed at various times after addition to the flasks, for example, after about: 2 h to 168 h, 12 h to 96 h, 24 h to 72 h, 36 h to 60 h, 42 h to 56 h, or 48 h, and then subsequently changed about: every day, every other day, every third day, every fifth day, once a week, once every two weeks or about less than once every two weeks. Through repeated removal of spent medium, the non-attached cells may be removed, thereby selecting the MSCs by their affinity for attachment to the Synthemax-treated surface. The cells may be cultured for a period of time, such as about, for example, 4 d, 7 d, 10 d, 11 d, 12 d, 13 d, 14 d, 18 d, 21 d, 28 d or longer than 21 d. Optionally, the cells may be cultured under hypoxic conditions: hypoxia priming may alter cell metabolism during expansion, increase resistance to oxidative stress, and thereby improve the engraftment, survival in ischemic microenvironments, and angiogenic potential of transplanted MSCs. After culturing, the PO colonies (Colony forming Units-CFUs) that have formed may be dissociated and pooled. After pooling, the remaining cells may be predominantly non-tissue specific MSCs. In certain examples, the pooled PO cells may be gently re-suspended in pre-warmed xeno-free cell culture media and re-plated on tissue-culture treated flasks without Synthemax for passaging. The pooled cells may be seeded at a seeding density of from between about: 100 to 10000 cells/cm², 500 to 8000 cells/cm², 1000 to 5000 cells/cm², or about 2000 to 4000 cells/cm². The media may be changed about every 1 d, 2 d, 4 d, or more than four days. After a period of time, such as about 2 d, 4 d, 7 d, or more than 7 d, the cells may be dissociated and harvested. Further selective MSC isolation may be achieved as described below.

### Identification of Markers

When comparing the genetic expression profiles of TAF MSCs and adult-type MSCs derived from adipose tissue or bone marrow by RNAseq, TAF MSCs tend to express more of some genes present in adult-type MSCs and less of others. Identification of both positive and negative TAF MSC specific neonatal cell-surface markers can allow for sorting of the MSCs with neonatal quality from those that have differentiated further and are of less importance as progenitor cells using e.g. ligands such as antibodies and aptamers or other selection techniques.

The cell surface markers distinguishing tissue relevant cells from other MSCs may be elucidated via a bioinformatics process utilizing a tissue-specificity score algorithm (as described in more detail in WO 2021/076042 A1 and WO 2021/076043 A1 supra, see FIG. 14 therein). Tissue-specificity may be measured as a combination of two components: a ‘tissue transcriptional similarity’ also known as a similarity score and a “tissue-specific gene expression program” also known as a gene set score. In certain examples, the similarity score may be an Average Spearman correlation to each MSC tissue reference sample (for example a fetal lung MSC sample). In examples, the gene set score may be the average expression of genes in a tissue-specific gene set. After normalizing the similarity and gene set scores using a Z-transform to convert the input values, which is a sequence of real or complex numbers, into a complex frequency-domain representation, then combining them assigning equal weight to each score and transforming combined values using a Z-transform, the resulting output is an MSC tissue specificity score. The MSC tissue-specificity score measures the relative tissue-specificity among the input samples by measuring how many standard deviations a sample is more or less specific to a given tissue compared to the average input sample. For example, an MSC tissue-specificity score may indicate how much more a clone sample appears to have a tissue specific phenotype, such as a lung phenotype, as compared to an average clone. Such an approach allows for identification of the top X % percentile scores using a normal distribution function, effectively the top X % of clones that are most tissue-specific to the relevant tissue.

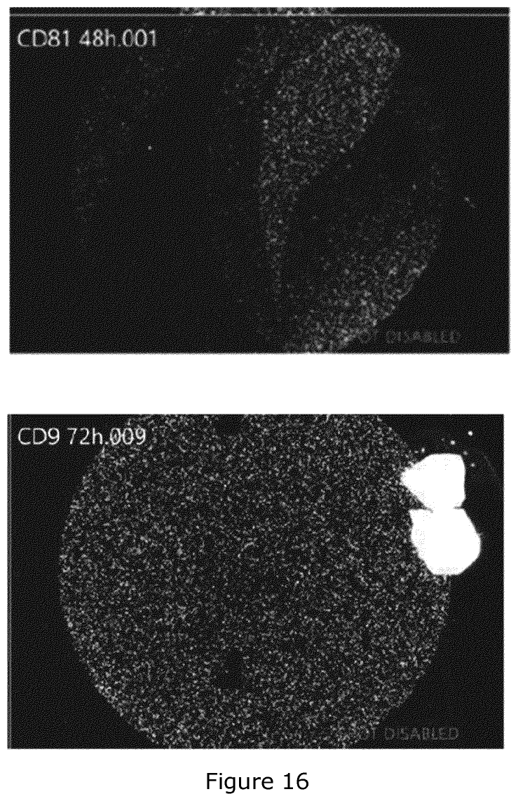

In one example, for a given tissue, tissue-prioritized clones can be defined as any clone belonging to the top X % percentile score, where X is any percentage within a range having a lower end from about 0.1 to 25, such as about 1, 5, 10, 15 and 20, and an upper end from about 30 to 75, such as about: 35, 40, 45, 50, 55, 60, 65 or 70. Having prioritized tissue-specific clones, candidate surface marker genes may then be identified. For each tissue, two groups may be defined: tissue-prioritized and tissue-distal. A suitable analysis program may be used to make this determination, for example DEseq2 from Bioconductor.org. The tissue-prioritized group may include clones with a score in the top 15% percentile. The tissue-distal group may include clones in the bottom Y % percentile in which Y is any percentage within the range having a lower end from about 25 to 70, such as about: 30, 35, 40, 45, 50, 55, 60 or 65 and an upper end from 75 to 99.9, such as about: 80, 85, 90, 95 or 99. FIG. 16 of WO 2021/076043 A1 shows an example of such analysis on kidney tissue. Next, differentially expressed genes between the tissue-prioritized and tissue-distal groups may be identified. Finally, the differential expression results may be annotated with surface marker gene information.

In certain examples, to identify tissue-specific cell surface markers, surface marker genes with a more than a Z-fold increase, where Z is at least about: 1.5-fold, 2-fold, 2.5-fold, 3-fold, 3.5-fold, 4-fold, 5-fold, 8-fold, 10-fold, 12-fold, 15-fold or even more-fold increase in expression (log 2FoldChange) in prioritized clones compared to an average clone and a Transcripts Per Kilobase Million (TPM) of more than about 500, such as more than about: 1000, 1500, 2000, 2500, 3000, 5000 or even higher may be selected to give the top tissue-specific marker candidates, such as approximately the top: 5, 10, 20, 30, 40, 50, 60, 70, 100 or more, for example such as those shown below in Tables 3-6 and further described in more detail below. Suitable log 2FoldChange and TPM values may vary even further depending on tissue type specificities depending on the abundance/absence of good markers.

Applying the tissue specificity algorithms described above to identify surface markers, after adhesion selection and passaging, the TAF MSCs cells may express various identified surface markers as shown below in Table 1, indicative of non-tissue specific TAF MSCs. One of skill in the art will understand that such surface markers may be present at various surface densities and may be upregulated or downregulated in comparison to other cell types. Therefore, such surface markers may be used to identify and isolate particular cell types. In some instances, the surface markers listed in Table 1 below may be at least 8-fold more highly expressed for TAF MSCs on average compared to other MSC cell types, particularly as compared to adult MSCs derived from bone marrow or adipose tissue. The thresholds used to generate Table 1 are as follows: X was selected as 15%, Y was selected as 50%, Z was selected as 8-fold and a TPM of more 3000 was selected. One of skill in the art will understand that the numbering used in Table 1 and all tables herein is merely used to indicate a total number of identified markers and not to indicate that one particular marker is more strongly expressed and/or preferred compared to another marker.

The groups of markers, including tissue-specific markers, for the present invention are as described in WO 2021/076042 A1 supra.

TABLE 1
Group A markers.

| | | |
|---|---|---|
| 1. | TBC1D3K | TBC1 domain family member 3K |
| 2. | AIF1L | allograft inflammatory factor 1 like |
| 3. | CDHR1 | cadherin related family member 1 |
| 4. | NKAIN4 | sodium/potassium transporting ATPase interacting 4 |
| 5. | ABCB1 | ATP binding cassette subfamily B member 1 |
| 6. | PLVAP | plasmalemma vesicle associated protein |
| 7. | MSLN | mesothelin |
| 8. | L1CAM | L1 cell adhesion molecule |
| 9. | HAVCR1 | hepatitis A virus cellular receptor 1 |
| 10. | MAL2 | mal, T cell differentiation protein 2 (gene/pseudogene) |
| 11. | SLAMF7 | SLAM family member 7 |
| 12. | DOC2B | double C2 domain beta |
| 13. | ESAM | endothelial cell adhesion molecule |
| 14. | GABRB1 | gamma-aminobutyric acid type A receptor beta1 ∝ |
| | | subunit |
| 15. | CDH16 | cadherin 16 |
| 16. | IGSF3 | immunoglobulin superfamily member 3 |
| 17. | DSC3 | desmocollin 3 |
| 18. | RHEX | regulator of hemoglobinization and erythroid cell |
| | | expansion |
| 19. | KCNIP1 | potassium voltage-gated channel interacting protein 1 |
| 20. | CD70 | CD70 molecule |
| 21. | GFRA1 | GDNF family receptor alpha 1 |
| 22. | CRB3 | crumbs cell polarity complex component 3 |
| 23. | CLDN1 | claudin 1 |
| 24. | AC118754.1 | novel transcript |
| 25. | SCN5A | sodium voltage-gated channel alpha subunit 5 |
| 26. | FGFR4 | fibroblast growth factor receptor 4 |
| 27. | KCNK3 | potassium two pore domain channel subfamily K |
| | | member 3 |
| 28. | DYSF | dysferlin |
| 29. | EFNA1 | ephrin A1 |
| 30. | KCNJ16 | potassium inwardly rectifying channel subfamily J |
| | | member 16 |
| 31. | MARCHF1 | membrane associated ring-CH-type finger 1 |
| 32. | SYTL1 | synaptotagmin like 1 |
| 33. | CLSTN2 | calsyntenin 2 |
| 34. | ITGB4 | integrin subunit beta 4 |
| 35. | VAMP8 | vesicle associated membrane protein 8 |
| 36. | GPRC5C | G protein-coupled receptor class C group 5 member C |
| 37. | CD24 | CD24 molecule |
| 38. | CELSR2 | cadherin EGF LAG seven-pass G-type receptor 2 |
| 39. | CDH8 | cadherin 8 |
| 40. | GRIP1 | glutamate receptor interacting protein 1 |
| 41. | DMTN | dematin actin binding protein |
| 42. | F11R | F11 receptor |
| 43. | CADM1 | cell adhesion molecule 1 |
| 44. | CDH6 | cadherin 6 |
| 45. | F2RL2 | coagulation factor II thrombin receptor like 2 |
| 46. | LYPD1 | LY6/PLAUR domain containing 1 |
| 47. | SLC6A6 | solute carrier family 6 member 6 |
| 48. | DSG2 | desmoglein 2 |
| 49. | ADGRG1 | adhesion G protein-coupled receptor G1 |
| 50. | CCKAR | cholecystokinin A receptor |
| 51. | OXTR | oxytocin receptor |
| 52. | ITGA3 | integrin subunit alpha 3 |
| 53. | AMIGO2 | adhesion molecule with Ig like domain 2 |
| 54. | CELSR1 | cadherin EGF LAG seven-pass G-type receptor 1 |
| 55. | EPHB2 | EPH receptor B2 |

As will be understood by one of skill in the art, suitable combinations of the markers listed in Table 1 may be used to separate TAF-MSCs from adult MSCs by selecting for specific markers from Table 1 or combinations of two, three, four, five, six or more markers from Table 1. In certain examples, TAF MSCs can be more specifically identified by identifying a combination of stronger expression, such as 8-fold or more stronger expression of any combination of the foregoing markers, e.g., TBC1D3K and/or AIF1L and/or CDHR1 and/or NKAIN4 and/or ABCB1 and/or PLVAP as compared to adult MSCs. When using combinations of markers, identification may be achieved with a lower threshold of stronger expression, such as 2-fold or more, 4-fold or more, or 6-fold or more expression of each of the markers.

In contrast to the above surface markers that may be more strongly expressed on the surface of TAF-MSCs (positive markers) compared to adult MSCs, in certain examples, the below surface markers in Table 2 may be more weakly expressed on TAF-MSCs as compared to other cell types (negative markers), such as ⅛-fold or less expression (optionally with TPM threshold >500) of any combination of the foregoing markers versus adult MSCs: IL13RA2, CLU, TMEM119, CEMIP, and LSP1. When using combinations of negative markers, identification may be achieved with a lower threshold of weaker expression, such as ½-fold or less, ¼-fold or less, or ⅙-fold or less expression of each of the markers.

Combinations of two or more these negative markers can also be used to more specifically isolate TAF MSCs. In addition, those skilled in the art will also recognize that combinations including both negative and positive markers, such as at any of the thresholds described above, can also be effective to more specifically isolate TAF MSCs.

TABLE 2
Markers that have reduced expression in TAF MSCs.

| | | |
|---|---|---|
| 1. | IL13RA2 | Interleukin-13 receptor subunit alpha-2 |
| 2. | CLU | Clusterin |
| 3. | TMEM119 | Transmembrane Protein 119 |
| 4. | CEMIP | Cell Migration Inducing Hyaluronidase 1 |
| 5. | LSP1 | Lymphocyte Specific Protein 1 |
| 6. | GPNMB | Glycoprotein Nmb |
| 7. | FAP | Fibroblast Activation Protein Alpha |
| 8. | CRLF1 | Cytokine Receptor Like Factor 1 |
| 9. | MME | Membrane Metalloendopeptidase |
| 10. | CLMP | CXADR Like Membrane Protein |
| 11. | BGN | Biglycan |
| 12. | DDR2 | Discoidin Domain Receptor Tyrosine Kinase 2 |

### Marker-Based Selection

Amniotic fluid contains heterogenous cells in a homogenous fluid. Hence, a marker-based selection may be needed. One example of marker-based selection is via the use of Fluorescence activated cell sorting (FACS). FACS may be used to purify the cell population of TAF-MSCs, FACS allows for a very high purity of the desired cell population, even when the target cell type expresses very low levels of identifying markers and/or separation is needed based on differences in marker density. FACS allows the purification of individual cells based on size, granularity and fluorescence. As will be understood by one of skill in the art, FACS may be used to select for certain cell populations that express one cell surface marker more than another cell population and vice-versa. In some examples of methods of purification, bulk methods of purification such as panning, complement depletion and magnetic bead separation, may be used in combination with FACS or as an alternative to FACS. In brief, to purify cells of interest via FACS, they are first stained with fluorescently-tagged monoclonal antibodies (mAbs), which recognize specific surface markers on the desired cell population. Negative selection of unstained cells may also allow for separation. For GMP production of cells according to some examples, FACS may be run using a closed system sorting technology such as MACSQuant® Tyto®. Samples may be kept contamination-free within the disposable, fully closed MACSQuant Tyto Cartridge. Further, filtered air may drive cells through a microchannel into the microchip at very low pressure (<3 PSI). However, before entering the microchannel, potential cell aggregates may be held back by a filter system guaranteeing a smooth sorting process. The fluorescence detection system may detect cells of interest based on predetermined fluorescent parameters of the cells. Based on their fluorescent and scatter light signatures, target cells may be redirected by a sort valve located within the microchannel. For certain examples of methods of purification, the success of staining and thereby sorting may depend largely on the selection of the identifying markers and the choice of mAb. Sorting parameters may be adjusted depending on the requirement of purity and yield. Unlike on conventional droplet sorters, cells sorted by the MACSQuant Tyto may not experience high pressure or charge, and may not get decompressed. Therefore, such a gentle sorting approach may result in high viability and functionality of cells. Alternatively, other marker-based selection techniques may be known to the skilled person and employed here. These include, but are not limited to, Magnetic-activated cell sorting, Microfluidic based sorting, Buoyancy activated cell sorting, mass cytometry etc.

### Tissue Specific Cells and Usage

### Lung TAF Cell Markers

As explained above, analysis of RNAseq data from TAF-MSC clones, adult and neonatal MSC reference material as well as fetal fibroblasts and publicly available expression datasets may be used to identify and characterize TAF-MSC cells. For example, sub-populations of TAF-MSCs may be established by clustering their expression data (RNAseq) with neonatal reference samples. Such sub-populations include, but are not limited to, lung MSC, urinary tract MSC (described also as kidney MSCs in the present disclosure), and skin MSC. Gene lists of highly and lowly expressed genes for each cluster of expression data may allow for identification of surface maker genes for each cluster. Using such data comparison, sub-populations of TAF cells were compared to adult MSC cells based on their gene expressions (RNAseq) resulting in a list of neonatal-specific surface marker genes for each cluster. A number of surface markers of interest associated with lung TAF cells were identified. For example, a non-exclusive list of preferred surface markers used to identify and separate lung TAF cells are provided below. Moreover, as the number of different MSC-subtypes in TAF is limited, the selection of the tissue specific MSC may be done by firstly characterization, thereafter a stepwise negative selection/sorting of the material by taking into account the combined (multivariate) surface marker profile of the different tissue specific MSC's. One of skill in the art will understand that any such combination of these surface markers may be used for identifying and isolation of lung TAF cells from the general population of TAF-derived cells and/or TAF-MSC cells. In some examples, the below non-exclusive list of surface markers may be more highly expressed on the surface of Lung-TAF cells as compared to other cell types, such as other TAF-derived cells and/or TAF-MSC cells.

As explained above, bioinformatics techniques may be used to identify tissue-specific surface markers, therefore, the surface markers identified in Table 3 may have at least a 10-fold increase in expression on prioritized clones compared to the average TAF-MSC clone (optionally with TPM threshold >2000).

TABLE 3
Group B markers.

| | | |
|---|---|---|
| 1. | PCDH19 | protocadherin 19; |
| 2. | DDR1 | discoidin domain receptor tyrosine kinase 1 |
| 3. | MME (CD10) | membrane metalloendopeptidase |
| 4. | IFITM10 | interferon induced transmembrane protein 10; |
| 5. | BGN | biglycan |
| 6. | NOTCH3 | notch receptor 3; |
| 7. | SULF1 | sulfatase 1; |
| 8. | TNFSF18 | TNF superfamily member 18; |
| 9. | BDKRB1 | bradykinin receptor B1; |
| 10. | FLT1 | fms related tyrosine kinase 1 |
| 11. | PDGFRA | platelet derived growth factor receptor alpha; |
| 12. | TNFSF4 | TNF superfamily member 4; |
| 13. | UNC5B | unc-5 netrin receptor B; |
| 14. | FAP | fibroblast activation protein alpha |
| 15. | CASP1 | caspase 1; |
| 16. | CD248 | Endosialin; |
| 17. | DDR2 | discoidin domain receptor tyrosine kinase 2 |
| 18. | PCDH18 | protocadherin 18; and/or |
| 19. | CRLF1 | cytokine receptor like factor 1; |

In contrast to the above surface markers that may be more strongly expressed on the surface of lung TAF MSCs, in certain examples, the below surface markers may be more weakly expressed on lung TAF MSCs as compared to other cell types, such as other TAF-derived cells and/or TAF-MSCs: CD24, ITGB4, TNFSF10, GFRA1, CD74, FGFR4, HAVCR1, and OSCAR. As will be understood by one of skill in the art, one, two, three, four, or more of the aforementioned more weakly expressed surface markers may be used to separate lung TAF cells from other cell types such as other TAF-derived cells and/or TAF-MSCs.

In certain examples, the cell surface marker CD248 (Endosialin) may be used to sort lung TAF MSCs from a population of TAF MSCs. Further surface markers that may be used to sort lung TAF MSCs include DDR-1 (discoidin domain receptor tyrosine kinase 1) as well as LRRC38 (Leucine Rich Repeat Containing Protein 38), all three of which have been identified via antibodies as useful markers for separation. In some examples, Endosialin, DDR-1, and/or LRRC38 alone or in combination with other markers may be used to sort. Endosialin may be combined with DDR-1 or LRRC38 to sort, or DDR-1 and LRRC38 may be combined without Endosialin.

As will be understood by one of skill in the art, suitable combinations of the markers listed in Table 3 and CD248, DDR-1, and LRR38 may be used to separate lung TAF MSCs from TAF MSCs by selecting for specific markers from Table 3 or combinations of two, three, four, five, six or more markers from Table 3 and/or CD248 and/or DDR-1 and/or LRR38. In certain examples, lung TAF MSCs can be more specifically identified by identifying a combination of stronger expression, such as 10-fold or more stronger expression (optionally with TPM threshold >2000) of any combination of the foregoing markers, e.g., PCDH19 and/or DDR1 and/or MME and/or IFITM10 and/or BGN and/or NOTCH3 and/or CD248 and/or DDR-1 and/or LRR38 as compared to TAF MSCs. When using combinations of markers, identification may be achieved with a lower threshold of stronger expression, such as 4-fold or more, 6-fold or more, or 8-fold or more expression of each of the markers.

In contrast to the above surface markers that may be more strongly expressed on the surface of lung TAF MSCs (positive markers) compared to TAF MSCs, in certain examples, the below surface markers may be more weakly expressed on lung TAF-MSCs as compared to other cell types (negative markers), such as ⅛-fold or less expression (optionally with TPM>500) of any combination of the foregoing markers versus TAF MSCs: CD24, ITGB4, TNFSF10, GFRA1, CD74, FGFR4, HAVCR1, and OSCAR. When using combinations of negative markers, identification may be achieved with a lower threshold of weaker expression, such as ½-fold or less, ¼-fold or less, or ⅙-fold or less expression of each of the markers.

Combinations of two or more these negative markers can also be used to more specifically isolate lung TAF MSCs. In addition, those skilled in the art will also recognize that combinations including both negative and positive markers, such as at any of the thresholds described above, can also be effective to more specifically isolate lung TAF MSCs.

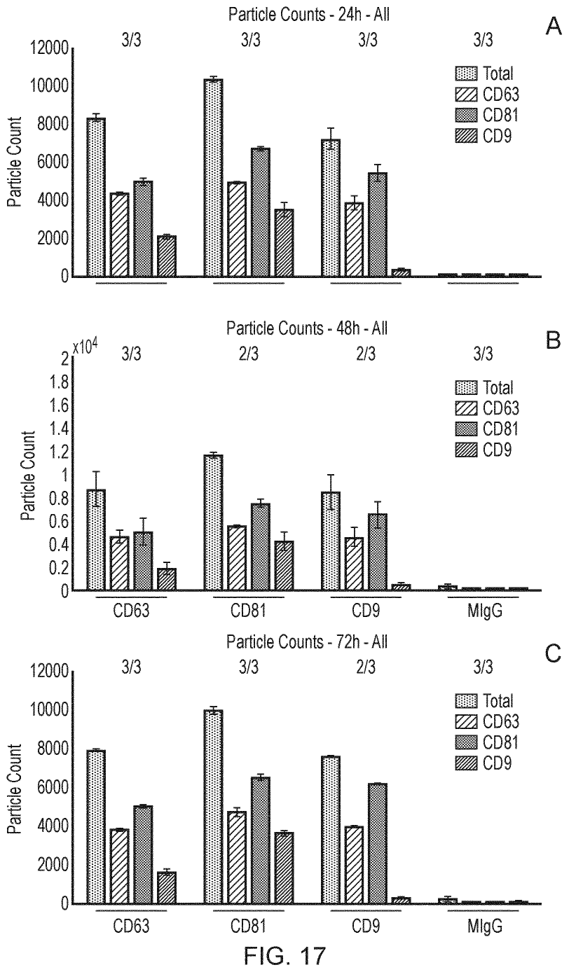

FIGS. 17A-17D of WO 2021/076043 A1 show an example of the results from a proof-of-principle study on the potential use of Lung TAF MSCs for treatment, performed using neonatally sorted TAF MSCs expressing MSC lung cell surface markers including CD248, DDR1, and LRRC38 (called “LBX-THX-001 cells”). The purpose of the study was to investigate the effects of LBX-THX-001 cells in a bleomycin induced lung fibrosis model in male rats. Two cell concentrations (2 M cell/kg and 5 M cells/kg) and two types of vehicles for the cells were tested (PBS and CryoStor CS-10).

The development of fibrosis in rat lung after exposure to bleomycin is well documented in the literature and a frequently used model for studying the pathology of lung fibrosis and also the effect of different treatments. The number of LBX-THX-001 cells injected were chosen to be relevant for a possible human therapy. The number of cells were therefore chosen to reflect cell numbers used in previous studies on rats (8-20 M cells/kg) and humans (0.5-2 M cells/kg).

An intra-tracheal instillation of bleomycin (1000 U/rat) to 34 male SD-rats was used to induce lung fibrosis in the rats. During the first week, the rats were monitored and weighed daily and thereafter twice/week until termination of the study. At day 4 post bleomycin challenge, the LBX-THX-001 cells were administered by an intravenous (i.v.) injection. The injection volume was 194-535 μL (maximal tolerated injection volume 1 mL/kg). The response to the intra-tracheal instillation of bleomycin was as expected based on previous experience for the model with weight loss during the first days after instillation and thereafter recovery. There were no significant differences in weight loss between the bleomycin group and the treatment groups.

### Kidney TAF Cell Markers

Similar to the lung TAF MSC cell markers identified above, a number of surface markers of interest associated with kidney TAF cells were identified. For example, a non-exclusive list of surface markers used to identify and separate kidney TAF MSCs are provided below in Table 4. Similar to the lung TAF MSC markers, the surface markers identified in Table 4 may have at least a 12-fold increase in expression on prioritized kidney TAF clones compared to the average TAF-MSC clone (optionally with TPM threshold >2000). Moreover, as the number of different MSC-subtypes in TAF is limited, the selection of the tissue specific MSCs may be done first by characterization, and thereafter by a stepwise negative selection/sorting of the material by taking into account the combined (multivariate) surface marker profile of the different tissue specific MSC's. One of skill in the art will understand that any such combination of these surface markers may be used for identifying and isolation of kidney TAF cells from the general population of TAF-derived cells and/or TAF-MSC cells. In some examples, the below non-exclusive list of surface markers may be more highly expressed on the surface of kidney-TAF cells as compared to other cell types, such as other TAF-derived cells and/or TAF-MSC cells:

TABLE 4
Group C markers.

| | | |
|---|---|---|
| 1. | HAVCR1 | hepatitis A virus cellular receptor 1; |
| 2. | CD24 | CD24 molecule |
| 3. | CLDN6 | claudin 6; |
| 4. | ABCB1 | ATP binding cassette subfamily B member 1; |
| 5. | SHISA9 | shisa family member 9; |
| 6. | CRB3 | crumbs cell polarity complex component 3 |
| 7. | AC118754.1 | Arachidonate 15-lipoxygenase, ALOX15, Smoothelin- |
| | | like protein 2, SMTNL2, Glutathione hydrolase 6, |
| | | GGT6, Myb-binding protein 1A, MYBBP1A, Protein |
| | | spinster homolog 2, SPNS2 |
| 8. | ITGB6 | integrin subunit beta 6; |
| 9. | CDH1 | cadherin 1 |
| 10. | LSR | lipolysis stimulated lipoprotein receptor |
| 11. | EPCAM | epithelial cell adhesion molecule; |
| 12. | AJAP1 | adherens junctions associated protein 1; |
| 13. | ANO9 | anoctamin 9 |
| 14. | CLDN7 | claudin 7; |
| 15. | EFNA1 | ephrin A1; |
| 16. | MAL2 | mal, T cell differentiation protein 2 (gene/pseudogene) |
| 17. | F11R | F11 receptor |
| 18. | L1CAM | L1 cell adhesion molecule; |
| 19. | GFRA1 | GDNF family receptor alpha 1; |
| 20. | IGSF3 | immunoglobulin superfamily member 3; |
| 21. | TNF | tumor necrosis factor |
| 22. | MMP7 | matrix metallopeptidase 7; |
| 23. | FOLR1 | folate receptor alpha; |
| 24. | TGFA | transforming growth factor alpha |
| 25. | C3 | complement C3 |
| 26. | TNFSF10 | TNF superfamily member 10; |
| 27. | PDGFB | platelet derived growth factor subunit B; and/or |
| 28. | WWC1 | WW and C2 domain containing 1 |

As will be understood by one of skill in the art, suitable combinations of the markers listed in Table 4 may be used to separate kidney TAF cells from TAF-MSCs by selecting for specific markers from Table 4 or combinations of two, three, four, five, six or more markers from Table 4. In certain examples, kidney TAF MSCs can be more specifically identified by identifying a combination of stronger expression, such as 12-fold or more stronger expression (optionally with TPM threshold >2000) of any combination of the foregoing markers, e.g., HAVCR1 and/or CD24 and/or CLDN6 and/or ABCB1 and/or SHISA9 and/or CRB3 as compared to TAF-MSCs. When using combinations of markers, identification may be achieved with a lower threshold of stronger expression, such as 4-fold or more, 6-fold or more, or 8-fold or more expression of each of the markers.

In contrast to the above surface markers that may be more strongly expressed on the surface of kidney TAF MSCs (positive markers), in certain examples, the below surface markers may be more weakly expressed on kidney TAF cells as compared to other cell types (negative markers), such as such as ⅛-fold or less expression (optionally with TPM threshold >500) of any combination of the foregoing markers other TAF-derived cells and/or TAF-MSC cells: GREM1, PDGFRB, BGN, FAP, CXCL12, CCKAR, CD248. When using combinations of negative markers, identification may be achieved with a lower threshold of weaker expression, such as ½-fold or less, ¼-fold or less, or ⅙-fold or less expression of each of the markers.

Combinations of two or more these negative markers can also be used to more specifically isolate kidney TAF MSCs. In addition, those skilled in the art will also recognize that combinations including both negative and positive markers, such as at any of the thresholds described above, can also be effective to more specifically isolate kidney TAF MSCs.

### Skin TAF Cell Markers

Similar to the lung and kidney TAF MSC markers identified above, a number of surface markers of interest associated with skin TAF cells were identified. For example, a non-exclusive list of surface markers used to identify and separate skin TAF cells are provided below in Table 5. The skin TAF MSC markers identified in Table 5 may have at least a 12-fold increase in expression on prioritized clones compared to the average TAF-MSC clone (optionally with TPM threshold >2000). Moreover, as the number of different MSC-subtypes in TAF is limited, the selection of the tissue specific MSC may be done by firstly characterization, thereafter a stepwise negative selection/sorting of the material by taking into account the combined (multivariate) surface marker profile of the different tissue specific MSC's. One of skill in the art will understand that any such combination of these surface markers may be used for identifying and isolation of skin TAF cells from the general population of TAF-derived cells and/or TAF-MSC cells. In some examples, the below non-exclusive list of surface markers may be more highly expressed on the surface of skin-TAF cells as compared to other cell types, such as other TAF-derived cells and/or TAF-MSC cells:

TABLE 5
Group D markers.

| | | |
|---|---|---|
| 1. | TNFSF18 | TNF superfamily member 18; |
| 2. | PCDH19 | protocadherin 19; |
| 3. | NCAM2 | neural cell adhesion molecule 2; |
| 4. | TNFSF4 | TNF superfamily member 4; |
| 5. | CD248 | Endosialin; |
| 6. | DDR2 | discoidin domain receptor tyrosine kinase 2 |
| 7. | HTR2B | 5-hydroxytryptamine receptor 2B; |
| 8. | PCDH18 | protocadherin 18; |
| 9. | SULF1 | sulfatase 1; |
| 10. | MME | membrane metalloendopeptidase |
| 11. | ADGRA2 | adhesion G protein-coupled receptor A2; |
| 12. | DCSTAMP | dendrocyte expressed seven transmembrane protein; |
| 13. | PDGFRA | platelet derived growth factor receptor alpha; |
| 14. | UNC5B | unc-5 netrin receptor B; |
| 15. | SCUBE3 | signal peptide, CUB domain and EGF like domain |
| | | containing 3; |
| 16. | CEMIP | cell migration inducing hyaluronidase 1; |
| 17. | BDKRB1 | bradykinin receptor B1; |
| 18. | FLT1 | fms related tyrosine kinase 1 |
| 19. | BDKRB2 | bradykinin receptor B2; |
| 20. | FAP | fibroblast activation protein alpha |
| 21. | CASP1 | caspase 1; and/or |
| 22. | SRPX2 | sushi repeat containing protein X-linked 2 |

As will be understood by one of skill in the art, suitable combinations of the markers listed in Table 5 may be used to separate skin TAF MSCs from TAF-MSCs by selecting for specific markers from Table 5 or combinations of two, three, four, five, six or more markers from Table 5. In certain examples, skin TAF MSCs can be more specifically identified by identifying a combination of stronger expression, such as 12-fold or more stronger expression (optionally with TPM>2000) of any combination of the foregoing markers, e.g., TNFSF18 and/or PCDH19 and/or NCAM2 and/or TNFSF4 and/or CD248 and/or DDR2 as compared to TAF-MSCs. When using combinations of markers, identification may be achieved with a lower threshold of stronger expression, such as 4-fold or more, 6-fold or more, or 8-fold or more expression of each of the markers.

In contrast to the above surface markers that may be more strongly expressed on the surface of skin TAF cells (positive markers), in certain examples, the below surface markers may be more weakly expressed on skin TAF cells as compared to other cell types (negative markers), such as such as ⅛-fold or less expression (optionally with TPM threshold >500) of any combination of the foregoing markers other TAF-derived cells and/or TAF-MSC cells: CD24, TNFSF10, ITGB4, ABCB1. When using combinations of negative markers, identification may be achieved with a lower threshold of weaker expression, such as ½-fold or less, ¼-fold or less, or ⅙-fold or less expression of each of the markers.

Combinations of two or more these negative markers can also be used to more specifically isolate skin TAF MSCs. In addition, those skilled in the art will also recognize that combinations including both negative and positive markers, such as at any of the thresholds described above, can also be effective to more specifically isolate skin TAF MSCs.

### Neural TAF Cell Markers

Similar to the lung, kidney, and skin TAF MSC markers identified above, a number of surface markers of interest associated with neural TAF cells were identified. For example, a non-exclusive list of surface markers used to identify and separate neural TAF cells are provided below. The neural TAF MSC surface markers identified in Table 6 may have at least a 3-fold increase in expression on prioritized clones compared to the average TAF-MSC clone (optionally with TPM threshold >500). Moreover, as the number of different MSC-subtypes in TAF is limited, the selection of the tissue specific MSC may be done by firstly characterization, thereafter a stepwise negative selection/sorting of the material by taking into account the combined (multivariate) surface marker profile of the different tissue specific MSC's. One of skill in the art will understand that any such combination of these surface markers may be used for identifying and isolation of neural TAF cells from the general population of TAF-derived cells and/or TAF-MSC cells. In some examples, the below non-exclusive list of surface markers may be more highly expressed on the surface of neural-TAF cells as compared to other cell types, such as other TAF-derived cells and/or TAF-MSC cells:

TABLE 6
Group E markers.

| | | |
|---|---|---|
| 1. | HAVCR1 | hepatitis A virus cellular receptor 1; |
| 2. | ACKR3 | atypical chemokine receptor 3; |
| 3. | OSCAR | osteoclast associated Ig-like receptor; |
| 4. | C3 | complement C3 |
| 5. | SIRPB1 | signal regulatory protein beta 1; |
| 6. | SLC6A6 | solute carrier family 6 member 6; |
| 7. | CCKAR | cholecystokinin A receptor; |
| 8. | TNFSF10 | TNF superfamily member 10; |
| 9. | CLSTN2 | calsyntenin 2; |
| 10. | TENM2 | teneurin transmembrane protein 2; |
| 11. | SFRP1 | secreted frizzled related protein 1; |
| 12. | PIK3IP1 | phosphoinositide-3-kinase interacting protein 1; |
| 13. | SCNN1D | sodium channel epithelial 1 delta subunit; |
| 14. | CLDN11 | claudin 11; |
| 15. | ALDH3B1 | aldehyde dehydrogenase 3 family member B1; and/or |
| 16. | ITGB4 | integrin subunit beta 4 |

As will be understood by one of skill in the art, suitable combinations of the markers listed in Table 6 may be used to separate neural TAF MSCs from TAF-MSCs by selecting for specific markers from Table 6 or combinations of two, three, four, five, six or more markers from Table 6. In certain examples, neural TAF MSCs can be more specifically identified by identifying a combination of stronger expression, such as 3-fold or more stronger expression (optionally with TPM threshold >500) of any combination of the foregoing markers, e.g., HAVCR1 and/or ACKR3 and/or OSCAR and/or C3 and/or SIRPB1 and/or SLC6A6 as compared to TAF-MSCs. When using combinations of markers, identification may be achieved with a lower threshold of stronger expression, such as 2-fold or more or a higher threshold such as 6-fold or more, 8-fold or more, or 12-fold or more expression of each of the markers. In addition, those skilled in the art will also recognize that combinations including both negative and positive markers, such as at any of the thresholds described above, can also be effective to more specifically isolate neural TAF MSCs.

Extracellular vesicles (EVs) from mesenchymal stem cells may be obtained from amniotic fluid by a method comprising: providing term amniotic fluid (TAF); removing particulate material from the TAF to obtain purified TAF cells; performing adherence selection on the purified TAF cells to obtain TAF adherence cells; passaging the TAF adherence cells to obtain TAF mesenchymal stem cells (TAF MSCs); propagating the TAF MSCs; and obtaining EVs from the TAF MSCS.

In some embodiments, the method further comprises a selecting step, wherein the TAF MSCs are selected based on the expression of a surface marker. Accordingly, in some embodiments, the method further comprises selecting TAF MSCs that express a marker selected from the group consisting of TBC1 domain family member 3K (TBC1D3K), allograft inflammatory factor 1 like (AIF1L), cadherin related family member 1 (CDHR1), sodium/potassium transporting ATPase interacting 4 (NKAIN4), ATP binding cassette subfamily B member 1 (ABCB1), plasmalemma vesicle associated protein (PLVAP), mesothelin (MSLN), L1 cell adhesion molecule (L1CAM), hepatitis A virus cellular receptor 1 (HAVCR1), mal, T cell differentiation protein 2 (gene/pseudogene) (MAL2), SLAM family member 7 (SLAMF7), double C2 domain beta (DOC2B), endothelial cell adhesion molecule (ESAM), gamma-aminobutyric acid type A receptor beta1 subunit (GABRB1), cadherin 16 (CDH16), immunoglobulin superfamily member 3 (IGSF3), desmocollin 3 (DSC3), regulator of hemoglobinization and erythroid cell expansion (RHEX), potassium voltage-gated channel interacting protein 1 (KCNIP1), CD70 molecule (CD70), GDNF family receptor alpha 1 (GFRA1), crumbs cell polarity complex component claudin 3 (CRB3), 1 (CLDN1), novel transcript (AC118754.1), sodium voltage-gated channel alpha subunit 5 (SCN5A), fibroblast growth factor receptor 4 (FGFR4), potassium two pore domain channel subfamily K member 3 (KCNK3), dysferlin (DYSF), ephrin A1 (EFNA1), potassium inwardly rectifying channel subfamily J member 16 (KCNJ16), membrane associated ring-CH-type finger 1 (MARCHF1), synaptotagmin like 1 (SYTL1), calsyntenin 2 (CLSTN2), integrin subunit beta 4 (ITGB4), vesicle associated membrane protein 8 (VAMP8), G protein-coupled receptor class C group 5 member C (GPRC5C), CD24 molecule (CD24), cadherin EGF LAG seven-pass G-type receptor 2 (CELSR2), cadherin 8 (CDH8), glutamate receptor interacting protein 1 (GRIP1), dematin actin binding protein (DMTN), F11 receptor (F11R), cell adhesion molecule 1 (CADM1), cadherin 6 (CDH6), coagulation factor II thrombin receptor like 2 (F2RL2), LY6/PLAUR domain containing 1 (LYPD1), solute carrier family 6 member 6 (SLC6A6), desmoglein 2 (DSG2), adhesion G protein-coupled receptor G1 (ADGRG1), cholecystokinin A receptor (CCKAR), oxytocin receptor (OXTR), integrin subunit alpha 3 (ITGA3), adhesion molecule with Ig like domain 2 (AMIGO2), cadherin EGF LAG seven-pass G-type receptor 1 (CELSR1), EPH receptor B2 (EPHB2). In another aspect, the isolated EVs are obtainable by the method according to the present disclosure, said cells expressing a surface marker selected from the group comprising of TBC1 domain family member 3K (TBC1D3K), allograft inflammatory factor 1 like (AIF1L), cadherin related family member 1 (CDHR1), sodium/potassium transporting ATPase interacting 4 (NKAIN4), ATP binding cassette subfamily B member 1 (ABCB1), plasmalemma vesicle associated protein (PLVAP), mesothelin (MSLN), L1 cell adhesion molecule (L1CAM), hepatitis A virus cellular receptor 1 (HAVCR1), mal, T cell differentiation protein 2 (gene/pseudogene) (MAL2), SLAM family member 7 (SLAMF7), double C2 domain beta (DOC2B), endothelial cell adhesion molecule (ESAM), gamma-aminobutyric acid type A receptor beta1 subunit (GABRB1), cadherin 16 (CDH16), immunoglobulin superfamily member 3 (IGSF3), desmocollin 3 (DSC3), regulator of hemoglobinization and erythroid cell expansion (RHEX), potassium voltage-gated channel interacting protein 1 (KCNIP1), CD70 molecule (CD70), GDNF family receptor alpha 1 (GFRA1), crumbs cell polarity complex component 3 (CRB3), claudin 1 (CLDN1), novel transcript (AC118754.1), sodium voltage-gated channel alpha subunit 5 (SCN5A), fibroblast growth factor receptor 4 (FGFR4), potassium two pore domain channel subfamily K member 3 (KCNK3), dysferlin (DYSF), ephrin A1 (EFNA1), potassium inwardly rectifying channel subfamily J member 16 (KCNJ16), membrane associated ring-CH-type finger 1 (MARCHF1), synaptotagmin like 1 (SYTL1), calsyntenin 2 (CLSTN2), integrin subunit beta 4 (ITGB4), vesicle associated membrane protein 8 (VAMP8), G protein-coupled receptor class C group 5 member C (GPRC5C), CD24 molecule (CD24), cadherin EGF LAG seven-pass G-type receptor 2 (CELSR2), cadherin 8 (CDH8), glutamate receptor interacting protein 1 (GRIP1), dematin actin binding protein (DMTN), F11 receptor (F11R), cell adhesion molecule 1 (CADM1), cadherin 6 (CDH6), coagulation factor II thrombin receptor like 2 (F2RL2), LY6/PLAUR domain containing 1 (LYPD1), solute carrier family 6 member 6 (SLC6A6), desmoglein 2 (DSG2), adhesion G protein-coupled receptor G1 (ADGRG1), cholecystokinin A receptor (CCKAR), oxytocin receptor (OXTR), integrin subunit alpha 3 (ITGA3), adhesion molecule with Ig like domain 2 (AMIGO2), cadherin EGF LAG seven-pass G-type receptor 1 (CELSR1), EPH receptor B2 (EPHB2). Alternatively, or additionally, a method for obtaining TAF MSCs from term amniotic fluid may comprise: providing term amniotic fluid (TAF); removing particulate material from the TAF to obtain purified TAF cells; performing adherence selection on the purified TAF cells to obtain TAF adherence cells; passaging the TAF adherence cells to obtain a population of cells comprising the TAF MSCs; and selecting the TAF MSCs from the population as cells that express at least one Group A surface marker selected from the group consisting of TBC1 domain family member 3K, allograft inflammatory factor 1 like, cadherin related family member 1, sodium/potassium transporting ATPase interacting 4, ATP binding cassette subfamily B member 1, plasmalemma vesicle associated protein, mesothelin, L1 cell adhesion molecule, hepatitis A virus cellular receptor 1, mal, T cell differentiation protein 2 (gene/pseudogene), SLAM family member 7, double C2 domain beta, endothelial cell adhesion molecule, gamma-aminobutyric acid type A receptor beta1 subunit, cadherin 16, immunoglobulin superfamily member 3, desmocollin 3, regulator of hemoglobinization and erythroid cell expansion, potassium voltage-gated channel interacting protein 1, CD70 molecule, GDNF family receptor alpha 1, crumbs cell polarity complex component 3, claudin 1, novel transcript sodium voltage-gated channel alpha subunit 5, fibroblast growth factor receptor 4, potassium two pore domain channel subfamily K member 3, dysferlin, ephrin A1, potassium inwardly rectifying channel subfamily J member 16, membrane associated ring-CH-type finger 1, synaptotagmin like 1, calsyntenin 2, integrin subunit beta 4, vesicle associated membrane protein 8, G protein-coupled receptor class C group 5 member C, CD24 molecule, cadherin EGF LAG seven-pass G-type receptor 2, cadherin 8, glutamate receptor interacting protein 1, dematin actin binding protein, F11 receptor, cell adhesion molecule 1, cadherin 6, coagulation factor II thrombin receptor like 2, LY6/PLAUR domain containing 1, solute carrier family 6 member 6, desmoglein 2, adhesion G protein-coupled receptor G1, cholecystokinin A receptor, oxytocin receptor, integrin subunit alpha 3, adhesion molecule with Ig like domain 2, cadherin EGF LAG seven-pass G-type receptor 1, and EPH receptor B2, thereby obtaining the TAF MSCs.

In some embodiments, selecting TAF MSCs may comprise selecting TAF MSCs that have a reduced expression of markers selected from the group consisting of IL13RA2, CLU, TMEM119, CEMIP, LSP1, GPNMB, FAP, MME (CD10), CRLF1, CLMP, BGN, DDR2. Removing particulate matter may comprise filtering and centrifuging the TAF. Performing adherence selection on the purified TAF cells may comprise adhering the purified TAF cells to a surface coated with a vitronectin-based substrate. As previously mentioned, the selecting step may be performed using FACS. The selecting step may be performed with antibodies directed to any of the markers or surface markers. The selecting step may comprise selecting TAF MSCs that express at least two markers from the Group A surface markers. The selecting step may comprise selecting TAF MSCs that express at least three markers from the Group A surface markers. The selecting step may comprise selecting TAF MSCs that express at least four markers from the Group A surface markers. The selecting step may comprise a plurality of sorting steps, each sorting step comprising directing TAF MSCs into a first output group or a second output group in dependence on a set of markers expressed or not expressed by the respective TAF MSCs.

In some embodiments, the selecting step may comprise a first sorting step to direct TAF MSCs that express a Group A surface marker into a first output group, and a second sorting step to direct TAF MSCs from the first output group that express a second set of markers into a second output group.

In certain embodiments, a method for obtaining EVs from term amniotic fluid lung mesenchymal stem cells (lung TAF MSCs) from term amniotic fluid, may comprise: providing term amniotic fluid (TAF); removing particulate material from the TAF to obtain purified TAF cells; performing adherence selection on the purified TAF cells to obtain TAF adherence cells; passaging the TAF adherence cells to obtain a population of cells comprising the lung TAF MSCs; selecting the TAF lung MSCs from the population as cells that express at least one Group B surface marker selected from the group consisting of PCDH19, DDR1, MME (CD10), IFITM10, BGN, NOTCH3, SULF1, TNFSF18, BDKRB1, FLT1, PDGFRA, TNFSF4, UNC5B, FAP, CASP1, CD248, DDR2, PCDH18, LRRC38, and CRLF1, thereby obtaining lung TAF MSCs; propagating the lung TAF MSCs; and obtaining EVs from the lung TAF MSCs.

Selecting lung TAF MSCs may comprise excluding MSCs that express a marker selected from the group consisting of CD24, ITGB4, TNFSF10, GFRA1, CD74, FGFR4, HAVCR1, and OSCAR. The selecting step may comprise selecting TAF MSCs that express at least two surface markers from the Group B surface markers. The selecting step may comprise selecting TAF MSCs that express at least three surface markers from the Group B surface markers. The selecting step may comprise selecting TAF MSCs that express at least four surface markers from the Group B surface markers. The selecting step may comprise selecting TAF MSCs that express a surface marker selected from the group of CD248, DDR1, and LRRC38. The selecting step may comprise selecting TAF MSCs that express CD248. The selecting step may comprise selecting TAF MSCs that express CD248 in combination with a marker selected from the group of DDR1 and LRRC38. The selecting step may comprise selecting TAF MSCs that express CD248, DDR1, and LRRC38. In some examples, isolated EVs may be obtainable by the methods described above, said cells expressing at least one Group A surface marker.

In some embodiments, EVs from an isolated population of TAF MSCs, may express at least one Group A surface marker selected from the group comprising of TBC1 domain family member 3K, allograft inflammatory factor 1 like, cadherin related family member 1, sodium/potassium transporting ATPase interacting 4, ATP binding cassette subfamily B member 1, plasmalemma vesicle associated protein, mesothelin, L1 cell adhesion molecule, hepatitis A virus cellular receptor 1, mal, T cell differentiation protein 2 (gene/pseudogene), SLAM family member 7, double C2 domain beta, endothelial cell adhesion molecule, gamma-aminobutyric acid type A receptor beta1 subunit, cadherin 16, immunoglobulin superfamily member 3, desmocollin 3, regulator of hemoglobinization and erythroid cell expansion, potassium voltage-gated channel interacting protein 1, CD70 molecule, GDNF family receptor alpha 1, crumbs cell polarity complex component 3, claudin 1, novel transcript sodium voltage-gated channel alpha subunit 5, fibroblast growth factor receptor 4, potassium two pore domain channel subfamily K member 3, dysferlin, ephrin A1, potassium inwardly rectifying channel subfamily J member 16, membrane associated ring-CH-type finger 1, synaptotagmin like 1, calsyntenin 2, integrin subunit beta 4, vesicle associated membrane protein 8, G protein-coupled receptor class C group 5 member C, CD24 molecule, cadherin EGF LAG seven-pass G-type receptor 2, cadherin 8, glutamate receptor interacting protein 1, dematin actin binding protein, F11 receptor, cell adhesion molecule 1, cadherin 6, coagulation factor II thrombin receptor like 2, LY6/PLAUR domain containing 1, solute carrier family 6 member 6, desmoglein 2, adhesion G protein-coupled receptor G1, cholecystokinin A receptor, oxytocin receptor, integrin subunit alpha 3, adhesion molecule with Ig like domain 2, cadherin EGF LAG seven-pass G-type receptor 1, and EPH receptor B2.

In some embodiments, a composition may comprise EVs from the isolated TAF MSCs described above and a pharmaceutically acceptable carrier for the TAF MSC-EVs. EVs from isolated lung TAF MSCs obtainable by a method described above may express at least one Group B surface marker selected from the group consisting of PCDH19, DDR1, MME (CD10), IFITM10, BGN, NOTCH3, SULF1, TNFSF18, BDKRB1, FLT1, PDGFRA, TNFSF4, UNC5B, FAP, CASP1, CD248, DDR2, PCDH18 and CRLF1. In certain examples, EVs from isolated lung TAF MSCs may express at least one Group B surface marker.

In some embodiments, a method for obtaining EVs from term amniotic fluid kidney mesenchymal stem (kidney TAF MSCs) cells, may comprise: providing term amniotic fluid (TAF); removing particulate material from the TAF to obtain purified TAF cells; performing adherence selection on the purified TAF cells to obtain TAF adherence cells; passaging the TAF adherence cells to obtain a population of cells comprising the TAF kidney MSCs; selecting the TAF kidney MSCs from the population as cells that express at least one Group C surface marker selected from the group consisting of HAVCR1, CD24, CLDN6, ABCB1, SHISA9, CRB3, AC118754.1, ITGB6, CDH1, LSR, EPCAM, AJAP1, ANO9, CLDN7, EFNA1, MAL2, F11R, L1CAM, GFRA1, IGSF3, TNF, MMP7, FOLR1, TGFA, C3, TNFSF10, PDGFB and WWC1, thereby obtaining kidney TAF MSCs; propagating the kidney TAF MSCs; and obtaining EVs from the kidney TAF MSCs.

In certain embodiments, EVs from isolated kidney TAF MSCs may express at least one Group C surface marker selected from the group consisting of HAVCR1, CD24, CLDN6, ABCB1, SHISA9, CRB3, AC118754.1, ITGB6, CDH1, LSR, EPCAM, AJAP1, ANO9, CLDN7, EFNA1, MAL2, F11R, L1CAM, GFRA1, IGSF3, TNF, MMP7, FOLR1, TGFA, C3, TNFSF10, PDGFB and WWC1. A composition may comprise EVs from the isolated kidney TAF MSCs described above and a pharmaceutically acceptable carrier for the kidney TAF MSC-EVs.

In some embodiments, a method for obtaining EVs from term amniotic fluid skin mesenchymal stem cells (skin TAF MSCs) may comprise: providing term amniotic fluid (TAF); removing particulate material from the TAF to obtain purified TAF cells; performing adherence selection on the purified TAF cells to obtain TAF adherence cells; passaging the TAF adherence cells to obtain a population of cells comprising the TAF skin MSCs; selecting the skin TAF MSCs from the population as cells that express at least one Group D surface marker selected from the group consisting of MME (CD10), TNFSF18, PCDH19, NCAM2, TNFSF4, CD248, DDR2, HTR2B, PCDH18, SULF1, ADGRA2, DCSTAMP, PDGFRA, UNC5B, SCUBE3, CEMIP, BDKRB1, FLT1, BDKRB2, FAP, CASP1, and SRPX2; and obtaining skin TAF MSCs; propagating the skin TAF MSCs; and obtaining EVs from the skin TAF MSCs. In some embodiments, the skin TAF MSCs are selected based on CD10 alone. In some embodiments, the EVs from skin TAF MSCs express CD10.

In certain embodiments, EVs from isolated skin TAF MSCs may express at least one Group D surface marker selected from the group consisting of TNFSF18, PCDH19, NCAM2, TNFSF4, CD248, DDR2, HTR2B, PCDH18, SULF1, MME (CD10), ADGRA2, DCSTAMP, PDGFRA, UNC5B, SCUBE3, CEMIP, BDKRB1, FLT1, BDKRB2, FAP, CASP1, and SRPX2. A composition may comprise EVs from the isolated skin TAF MSCs described above and a pharmaceutically acceptable carrier for the skin TAF MSC-EVs.

In some embodiments, a method for obtaining EVs from neural TAF MSCs may comprise: providing term amniotic fluid (TAF); removing particulate material from the TAF to obtain purified TAF cells; performing adherence selection on the purified TAF cells to obtain TAF adherence cells; passaging the TAF adherence cells to obtain a population of cells comprising the TAF neural MSCs; selecting the TAF neural MSCs from the population as cells that express at least one Group E surface marker selected from the group consisting of HAVCR1, ACKR3, OSCAR, C3, SIRPB1, SLC6A6, CCKAR, TNFSF10, CLSTN2, TENM2, SFRP1, PIK3IP1, SCNN1D, CLDN11, ALDH3B1, and ITGB4; thereby obtaining neural TAF MSCs; propagating the neural TAF MSCs; and obtaining EVs from the neural TAF MSCs.

In some embodiments, EVs from isolated neural TAF MSCs may express at least one Group E surface marker selected from the group consisting of HAVCR1, ACKR3, OSCAR, C3, SIRPB1, SLC6A6, CCKAR, TNFSF10, CLSTN2, TENM2, SFRP1, PIK3IP1, SCNN1D, CLDN11, ALDH3B1 and ITGB4. A composition may comprise EVs from the isolated neural TAF MSCs described above and a pharmaceutically acceptable carrier for the neural TAF MSC-EVs. In some embodiments, the isolated TAF MSCs have been pre-sorted or enriched to contain markers of interest using the techniques described herein. For example, the selecting step may enrich the population of TAF MSCs to comprise at least 10%, 20%, 30%, 40%, 50%, 60%, 70%, 80%, 90%, 95%, 96%, 97%, 98%, 99% or 100% skin TAF MSCs, lung TAF MSCs, kidney TAF MSCs, or neural TAF MSCs. In some embodiments, the remaining percentage of cells may be a mixture with another type of TAF MSCs, and/or with unsorted TAF MSCS.

In some embodiments, the isolated TAF MSCs have been passaged multiple times. For example, the isolated TAF MSCs may have been passaged 1, 2, 3, 4, 5, 6, or more times.

In some embodiments, the selecting step is performed after the passaging step and/or before the propagating step.

By “propagating” we include the meaning that the MSCs are cultured to allow their growth, thereby increasing the number of cells present in the culture. Due to the limited yield of EVs per MSC, it is advantageous, or even necessary, to include a propagating step to enhance the yield of EVs. The yield of EVs varies depending on the type of MSC used as a starting material, and so careful selection must be made to ensure an appropriate amount of EVs can be obtained.

In some embodiments, the method further comprises preconditioning the TAF MSCs. Preconditioning may be by serum-starvation, inflammation and/or hypoxia. In some embodiments, the preconditioning is by serum-starvation. Serum-starvation can be achieved by culturing cells in media that lacks supplementary serum (e.g. foetal calf serum, human serum albumin, and human platelet lysate).

In some embodiments, the extracellular vesicles are exosome vesicles and/or microvesicles. In some embodiments, the extracellular vesicles are not apoptotic vesicles. In some embodiments, the extracellular vesicles are between 20-1000 nm in diameter, for example between 50-600 nm or between 50-100 nm. In some embodiments, the extracellular vesicles are less than 1000 nm in diameter. In some embodiments, the extracellular vesicles are greater than 20 nm in diameter. In some embodiments, the diameter of the extracellular vesicles is assessed by Nanoparticle Tracking Analysis (NTA).

In some embodiments, the isolated extracellular vesicles are produced by any one or more of the methods described herein in accordance with the first aspect of the invention. In some embodiments, the isolated extracellular vesicles have a purity ratio that is based on an ExoView/ZetaView ratio. In some embodiments, the purity ratio is higher in comparison with isolated EVs from an adult stem cell source, for example adipose stem cells (ASCs), as based on an ExoView/ZetaView ratio. The ExoView/ZetaView ratio may be established based on an analysis of CD63, CD81 and/or CD9 (as demonstrated in the Examples herein). In some embodiments, the ExoView/ZetaView ratio of the isolated EVs may be:
1. with respect to CD63, at least 2.0E-03, such as 2.1E-03, 2.2E-03, 2.3E-03, 2.4E-03, 2.5E-03 or greater after 24 hours, 48 hours and/or 72 hours of serum-starvation for the TAF MSCs;
2. with respect to CD81, at least 2.7E-03, such as 2.8E-03, 2.9E-03, 3.0E-03, 3.1E-03, 3.2E-03, 3.3E-03 or greater after 24 hours, 48 hours and/or 72 hours of serum-starvation for the TAF MSCs; and/or
3. with respect to CD9, at least 2.0E-03, such as 2.1E-03, 2.2E-03, 2.3E-03, 2.4E-03 or greater after 24 hours, 48 hours and/or 72 hours of serum-starvation for the TAF MSCs.

In some embodiments, the isolated extracellular vesicles express at least one surface marker associated with skin TAF MSCs, lung TAF MSCs, kidney TAF MSCs, or neural TAF MSCs, as described herein.

In some embodiments, compositions, such as the decellularised compositions, comprising isolated EVs further comprises at least one pharmaceutically acceptable carrier, excipient or further component such as at least one therapeutic and/or prophylactic ingredient. A “pharmaceutically acceptable carrier” as referred to herein, is any known compound or combination of known compounds that are known to those skilled in the art to be useful in formulating pharmaceutical compositions. The carrier may include one or more excipients or diluents. Pharmaceutical compositions of the invention can be placed into dosage forms, such as in the form of unit dosages. Pharmaceutical compositions include those suitable for any route of administration (as discussed further below).

In some embodiments, the adverse immune response is inflammation. The isolated EVs or decellularised compositions described herein may be anti-inflammatory by reducing NFκB pathway signalling; reducing T helper cell activation (or reducing the number of activated T helper cells); increasing regulatory Treg numbers and/or activity; reducing total number of T cells or effector T cells; and/or reducing macrophage activation or the number of activation macrophages. Alternatively, or additionally, the isolated EVs or decellularised compositions described herein may be anti-inflammatory by modulating innate immune cells (such as neutrophils, macrophages, monocytes, fibrocytes, mast cells, innate lymphoid cells (ILCs; e.g. type 2 ILCs), and/or dendritic cells); and/or adaptive immune cells (such as Th1 cells, Th2 cells, Th9 cells, Th17 cells, Tregs, and/or B cells); and/or any of the inflammatory processes associated with each.

In some embodiments, the skin condition is a chronic skin ulcer (also referred to as a chronic cutaneous ulcer), a diabetic skin ulcer, or a bedsore.

In some embodiments, the fibrotic disease is scar formation (such as following a wound in the skin), liver cirrhosis, pulmonary fibrosis, renal interstitial fibrosis, myocardial infarction, systemic sclerosis (SSc), or graft-versus-host disease (GVHD).

In some embodiments, the cancer or tumour is selected from the group consisting of: Acute Lymphoblastic Leukemia (ALL); Acute Myeloid Leukemia (AML); Adrenocortical Carcinoma; AIDS-Related Cancers, also including for example Kaposi Sarcoma (Soft Tissue Sarcoma), AIDS-Related Lymphoma (Lymphoma), Primary CNS Lymphoma (Lymphoma); Anal Cancer; Appendix Cancer; Astrocytomas (Brain Cancer); Basal Cell Carcinoma of the Skin; Bile Duct Cancer; Bladder Cancer; Bone Cancer (for example, Ewing Sarcoma, Osteosarcoma or Malignant Fibrous Histiocytoma); Brain Tumours (including, for example, glioma or glioblastoma); Breast Cancer; Bronchial Tumours; Burkitt Lymphoma; Carcinoid Tumour (Gastrointestinal); Cardiac (Heart) Tumours; Cervical Cancer; Cholangiocarcinoma; Chordoma; Chronic Lymphocytic Leukemia (CLL); Chronic Myelogenous Leukemia (CML); Chronic Myeloproliferative Neoplasms; Colorectal Cancer; Endometrial Cancer (Uterine Cancer); Oesophageal Cancer; Esthesioneuroblastoma (Head and Neck Cancer); Eye Cancer; Gallbladder Cancer; Gastric (Stomach) Cancer; Gastrointestinal Carcinoid Tumour; Ovarian Germ Cell Tumours; Testicular Cancer; Kidney (Renal Cell) Cancer; Liver Cancer; Lung Cancer (e.g. Non-Small Cell and Small Cell); Melanoma; Prostate Cancer; Rectal Cancer; Vaginal Cancer; and Wilms Tumour.

In some embodiments, the cardiovascular disease is selected from the group consisting of: stroke, acute and chronic heart failure, atherosclerosis. In some embodiments, the arthritis is selected from the group consisting of: rheumatoid arthritis and osteoarthritis. In some embodiments, the bowel disease is selected from the group consisting of: Crohn's disease, inflammatory bowel disease, and inflammatory bowel syndrome.

In some embodiments, the condition to be treated or prevented is associated with the NFκB signalling pathway.

In some embodiments, the isolated EVs or decellularised composition comprising isolated EVs is for use in wound healing. For example, the isolated EVs or decellularised composition may accelerate the rate of wound healing following injury, wherein the acceleration is in comparison with an untreated control. The term “wound” or “wounding” includes, but is not limited to, injury, trauma, surgery, compromised skin and burns. The term “compromised skin” refers to skin which exhibits characteristics distinct from normal skin. Compromised skin may occur in association with a dermatological condition. Several non-limiting examples of dermatological conditions include rosacea, common acne, seborrheic dermatitis, perioral dermatitis, acneform rashes, transient acantholytic dermatosis, and acne necrotica miliaris. In some instances, compromised skin may comprise a wound and/or scar tissue. In some instances, methods and compositions associated with the invention may be used to promote wound healing, prevention, reduction or inhibition of scarring, and/or promotion of re-epithelialisation of wounds.

A therapeutically effective amount of isolated EVs or a decellularised composition described herein may in some embodiments be an amount sufficient to prevent the formation of compromised skin and/or improve the condition of compromised skin and/or to treat or prevent a fibrotic disorder. In some embodiments, improvement of the condition of compromised skin may correspond to promotion of wound healing and/or inhibition of scarring and/or promotion of epithelial regeneration. The extent of prevention of formation of compromised skin and/or improvement to the condition of compromised skin may in some instances be determined by, for example, a doctor or clinician.

The ability of isolated EVs or a decellularised compositions associated with the invention to prevent the formation of compromised skin and/or improve the condition of compromised skin may in some instances be measured with reference to properties exhibited by the skin. In some instances, these properties may include rate of epithelialisation and/or decreased size of an area of compromised skin compared to control skin at comparable time points.

As used herein, prevention of formation of compromised skin, for example prior to a surgical procedure, and/or improvement of the condition of compromised skin, for example after a surgical procedure, can encompass any increase in the rate of healing in the compromised skin as compared with the rate of healing occurring in a control sample. In some instances, the condition of compromised skin may be assessed with respect to either comparison of the rate of re-epithelialisation achieved in treated and control skin, or comparison of the relative areas of treated and control areas of compromised skin at comparable time points. In some embodiments, isolated EVs or decellularised compositions that prevent formation of compromised skin or promote healing of compromised skin may be isolated EVs or decellularised compositions that, upon administration, causes the area of compromised skin to exhibit an increased rate of re-epithelialisation and/or a reduction of the size of compromised skin compared to a control at comparable time points. In some embodiments, the healing of compromised skin may give rise to a rate of healing that is 5%, 10%, 20%, 30%, 40%, 50%, 60%, 70%, 80%, 90% or 100% greater than the rate occurring in controls.

In some embodiments, subjects to be treated by methods and compositions associated with the invention may be subjects who will undergo, are undergoing or have undergone a medical procedure such as a surgery. In some embodiments, the subject may be prone to defective, delayed or otherwise impaired re-epithelialisation, such as dermal wounds in the aged. Other non-limiting examples of conditions or disorders in which wound healing is associated with delayed or otherwise impaired re-epithelialisation include patients suffering from diabetes, patients with polypharmacy, post-menopausal women, patients susceptible to pressure injuries, patients with venous disease, clinically obese patients, patients receiving chemotherapy, patients receiving radiotherapy, patients receiving steroid treatment, and immuno-compromised patients. In some instances, defective re-epithelialisation response can contribute to infections at the wound site, and to the formation of chronic wounds such as ulcers.

In some embodiments, methods or uses associated with the invention may promote the re-epithelialisation of compromised skin in chronic wounds, such as ulcers, and may also inhibit scarring associated with wound healing. In other embodiments, methods or uses associated with the invention are applied to prevention or treatment of compromised skin in acute wounds in patients predisposed to impaired wound healing developing into chronic wounds. In other aspects, methods or uses associated with the invention are applied to promote accelerated healing of compromised skin while preventing, reducing or inhibiting scarring for use in general clinical contexts. In some embodiments, this can involve the treatment of surgical incisions and application of such methods may result in the prevention, reduction or inhibition of scarring that may otherwise occur on such healing. Such treatment may result in the scars being less noticeable and exhibiting regeneration of a more normal skin structure. In other embodiments, the compromised skin that is treated is not compromised skin that is caused by a surgical incision. The compromised skin may be subject to continued care and continued application of medicaments to encourage re-epithelialisation and healing.

In some embodiments, methods or uses associated with the invention may also be used in the treatment of compromised skin associated with grafting procedures. This can involve treatment at a graft donor site and/or at a graft recipient site. Grafts can in some embodiments involve skin, artificial skin, or skin substitutes. Methods associated with the invention can also be used for promoting epithelial regeneration. As used herein, promotion of epithelial regeneration encompasses any increase in the rate of epithelial regeneration as compared to the regeneration occurring in a control-treated or untreated epithelium. The rate of epithelial regeneration attained can in some instances be compared with that taking place in control-treated or untreated epithelia using any suitable model of epithelial regeneration known in the art. Promotion of epithelial regeneration may be of use to induce effective re-epithelialisation in contexts in which the re-epithelialisation response is impaired, inhibited, retarded or otherwise defective. Promotion of epithelial regeneration may be also affected to accelerate the rate of defective or normal epithelial regeneration responses in patients suffering from epithelial damage.

Some instances where re-epithelialisation response may be defective include conditions such as pemphigus, Hailey-Hailey disease (familial benign pemphigus), toxic epidermal necrolysis (TEN)/Lyell's syndrome, epidermolysis bullosa, cutaneous leishmaniasis and actinic keratosis. Defective re-epithelialisation of the lungs may be associated with idiopathic pulmonary fibrosis (IPF) or interstitial lung disease. Defective re-epithelialisation of the eye may be associated with conditions such as partial limbal stem cell deficiency or corneal erosions. Defective re-epithelialisation of the gastrointestinal tract or colon may be associated with conditions such as chronic anal fissures (fissure in ano), ulcerative colitis or Crohn's disease, and other inflammatory bowel disorders.

In some embodiments, methods or uses associated with the invention are used to prevent, reduce or otherwise inhibit compromised skin associated with scarring. This can be applied to any site within the body and any tissue or organ, including the skin, eye, nerves, tendons, ligaments, muscle, and oral cavity (including the lips and palate), as well as internal organs (such as the liver, heart, brain, abdominal cavity, pelvic cavity, thoracic cavity, guts and reproductive tissue). In the skin, treatment may change the morphology and organization of collagen fibers and may result in making the scars less visible and blend in with the surrounding skin. As used herein, prevention, reduction or inhibition of scarring encompasses any degree of prevention, reduction or inhibition in scarring as compared to the level of scarring occurring in a control-treated or untreated wound.

Prevention, reduction or inhibition of compromised skin, such as compromised skin associated with dermal scarring, can be assessed and/or measured with reference to microscopic and/or macroscopic characteristics. Macroscopic characteristics may include color, height, surface texture and stiffness of the skin. In some instances, prevention, reduction or inhibition of compromised skin may be demonstrated when the color, height, surface texture and stiffness of the skin resembles that of normal skin more closely after treatment than does a control that is untreated. Microscopic assessment of compromised skin may involve examining characteristics such as thickness and/or orientation and/or composition of the extracellular matrix (ECM) fibers, and cellularity of the compromised skin. In some instances, prevention, reduction or inhibition of compromised skin may be demonstrated when the thickness and/or orientation and/or composition of the extracellular matrix (ECM) fibers, and/or cellularity of the compromised skin resembles that of normal skin more closely after treatment than does a control that is untreated.

In some embodiments, methods or uses associated with the invention are for cosmetic purposes, at least in part to contribute to improving the cosmetic appearance of compromised skin. In some embodiments, methods associated with the invention may be used to prevent, reduce or inhibit compromised skin such as scarring of wounds covering joints of the body. In other embodiments, methods associated with the invention may be used to promote accelerated wound healing and/or prevent, reduce or inhibit scarring of wounds at increased risk of forming a contractile scar, and/or of wounds located at sites of high skin tension. Such methods or uses may also be involved with antiaging, for example promoting skin integrity in such a way that it has an appearance enhanced youth.

In some embodiments, methods or uses associated with the invention can be applied to promoting healing of compromised skin in instances where there is an increased risk of pathological scar formation, such as hypertrophic scars and keloids, which may have more pronounced deleterious effects than normal scarring. In some embodiments, methods or uses described herein for promoting accelerated healing of compromised skin and/or preventing, reducing or inhibiting scarring are applied to compromised skin produced by surgical revision of pathological scars.

Keloids are a particularly aggressive form of dermal scars that do not regress. Keloid scars are raised, irregular-shaped, pink to dark red in color and characteristically extend beyond the boundaries of the original wound. Keloids are commonly tender or painful and may itch intensely. While keloids are more prevalent in darker skinned individuals and often run in families, keloids can occur in people with all skin types. Current treatments are not satisfactory and include corticosteroid injections, cryotherapy, skin needling, pressure or silicone dressings, laser or radiation treatments and surgical removal. Since keloids form at the site of inflammation or injury, keloid treatments or removal may result in an even larger keloid.

In some embodiments, the isolated EVs or decellularised composition are/is administered between 72 hours prior to a wound and/or 24 hours after a wound. For example, the isolated EVs or decellularised composition may be administered approximately 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 or less than 1 hour before a wound. In other embodiments, the isolated EVs or decellularised composition may be administered approximately 1, 2, 3, 4, 5, 6, 7, 8, 9, 10, 11, 12, 13, 14, 15, 6, 17, 18, 19, 20, 21, 22, 23, 24 or more than 24 hours after a wound.

In some embodiments, administration or treatment is delayed. For example, the isolated EVs or decellularised composition may be administered 48 hours or more after a wound. In some embodiments, the isolated EVs or decellularised composition may be administered 48 hours (2 days), 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days or more than 30 days after a wound.

In some embodiments, the inflammation is selected from the group consisting of tissue-specific inflammation and organ-specific inflammation. In some embodiments, the inflammation may be in a tissue and/or organ selected from the group consisting of lung, kidney, neural, skin, liver, heart (and heart valves), trachea, body parts (such as limbs/digits), pancreas, intestine, colon and combinations thereof. Preferably, the inflammation to be treated is skin inflammation. In some embodiments, the inflammation may be systemic inflammation. In some embodiments, multiple types of inflammation may be occurring simultaneously. For example, the inflammation may be skin inflammation and heart inflammation, or skin inflammation and systemic inflammation.

In some embodiments, use in transplantation involve a donor tissue and/or donor organ obtained from a donor treated with isolated EVs. For example, the donor may have received isolated EVs or a composition comprising isolated EVs before (e.g. immediately before) the tissue or organ was retrieved from the donor. In some embodiments, the isolated EVs or composition comprising isolated EVs may be the same that the donor received as those used for the recipient of the donor tissue or organ. Alternatively, or additionally, the donor tissue and/or the donor organ may have been pre-treated ex-vivo with isolated EVs or a composition comprising isolated EVs. For example, the donor tissue and/or the donor organ may be removed from the donor and subsequently exposed to isolated EVs or a composition comprising isolated EVs prior to the donor tissue and/or the donor organ being transplanted into a recipient.

In some embodiments, the donor tissue and/or the donor organ has been transported ex-vivo in a conditioning media. Preferably, the conditioning media is a physiological conditioning media. The conditioning media may comprise isolated EVs and/or a composition comprising isolated EVs. In some embodiments, the conditioning media may further comprise one or more of the following components: dextran (e.g. dextran 40), red blood cells, and albumin (for example, human albumin). It may be appreciated that the conditioning media haematocrit (also referred to as the erythrocyte volume fraction) is at a concentration from 10 v/v % to 25v/v %, for example from 15v/v % to 25v/v %, or 10v/v %, 11v/v %, 12v/v %, 13v/v %, 14v/v %, 15v/v %, 16v/v %, 17v/v %, 18v/v %, 19v/v %, 20v/v %, 21v/v %, 22v/v %, 23v/v %, 24v/v %, or 25v/v %. In a preferred embodiment the conditioning media haematocrit is 14v/v %). Values considered normal for red blood cells in the blood are about 45v/v % for males and about 40v/v % for females. In some embodiments, albumin (e.g. human albumin, also referred to as human serum albumin (HSA)) is at a concentration from 1-25v/v %, for example 5-25v/v %, 1v/v %, 5v/v %, 10v/v %, 15v/v %, 20v/v %, or 25v/v %. In some embodiments, the conditioning media may further comprise one or more of the following components: at least one glucocorticoid (e.g. prednisolone and/or methylprednisolone), at least one anticoagulant (e.g. heparin), and at least one antibiotic. Conditioning media known in the art may be adapted to include TAF MSC-EVs as a supplement. For example, solutions used in EVLP, such as Steen™ solution.

In some embodiments, the isolated EVs comprise tissue-specific markers and/or organ-specific markers, preferably wherein the tissue-specific markers and/or the organ-specific markers correspond to said donor tissue or donor organ. In some embodiments, the isolated EVs may be a mixed population of multiple subtypes of isolated EVs, in which case a portion of the isolated EVs comprise tissue-specific markers and/or organ-specific markers while another portion comprise different tissue-specific markers and/or organ-specific markers, preferably wherein at least one of the portions of tissue-specific markers and/or the organ-specific markers correspond to said donor tissue or donor organ.

In some embodiments, the donor tissue and/or donor organ is from a non-living subject. Preferably, the non-living subject is the same species as the intended recipient of the donor tissue and/or donor organ. For example, the tissue and/or organ may be obtained from a non-living human (also referred to as a corpse or cadaver) and is for transplantation in a human in need thereof. In some embodiments, the donor tissue and/or donor organ is from a living subject. Preferably, the living subject is the same species as the intended recipient of the donor tissue and/or donor organ. A donor tissue and/or donor organ provided by a living subject is limited to a donor tissue and/or donor organ that can be parted with from the donor without resulting in cessation of the donor's life. For example, a single kidney of a functional pair of kidneys could be donated, or a skin graft taken from an excess of skin.

In some embodiments, ex-vivo donor tissue and/or ex-vivo donor organ may be selected from the group consisting of skin, a lung, kidney, neural, liver, heart (and heart valves), trachea, pancreas, intestine, colon and body parts. Body parts may be any body part such as limbs (e.g. arms and legs) or digits. In a preferred embodiment, the ex-vivo donor tissue and/or ex-vivo donor organ is skin. The ex-vivo donor tissue and/or ex-vivo donor organ may also be a portion of ex-vivo donor organs selected from the group consisting of skin, a lung, kidney, neural, liver, heart (and heart valves), trachea, pancreas, intestine, colon and body parts.

In the present context a tissue is a group of cells with a similar structure, organised to carry out specific functions. An organ is a collection of tissues that structurally form a functional unit specialised to perform a particular function. Accordingly, the term “portion thereof” with respect to an organ may refer to a tissue. Within the context of limbs and digits (in reattachment and/or re-enervation), the tissue and/or organ in question may be skin and/or a part of the nervous system. For example, reattachment of a digit may be a finger that has been separated from a subject, wherein the skin of the finger is reattached to the subject at the site from where it is lost. Alternatively, the digit may be from a donor, in which case it is attached in replacement of a limb or digit that a recipient has lost.

Even a small increase in the ex-vivo life of a donor organ and/or a donor tissue positively impacts the number of available transplantable organs as new geographical areas may be applied to supply donor organs and/or donor tissue. Thus, it may be preferred that the ex-vivo life of the ex-vivo donor tissue and/or ex-vivo donor organ is prolonged by at least 10 minutes, e.g. 20 minutes, such as 30 minutes, e.g. 40 minutes, such as 50 minutes, e.g. 1 hour, such as 2 hours compared to a control wherein the control is an ex-vivo donor tissue and/or ex-vivo donor organ not subjected to isolated EVs or a composition comprising isolated EVs.

In some embodiments, the ex-vivo donor tissue and/or ex-vivo donor organ remains viable outside the body for at least 1 hour, such as 2 hours, e.g. 4 hours, such as 6 hours, e.g. 8 hours, such as 10 hours, e.g. 12 hours, such as 14 hours, e.g. 16 hours, such as 18 hours, e.g. 20 hours, such as 22 hours, e.g. 1 days, such as 2 days. In the present context the term viability is to be understood as how long an ex-vivo donor tissue and/or ex-vivo donor organ can stay outside the body before the cell function begins to fail and the likelihood that the ex-vivo organ and/or ex-vivo tissue will malfunction in the recipient will increase. Transplant organ failure, known as primary graft dysfunction (PGD), is the “most feared complication” associated with organ transplants. Alternatively, or additionally, transplant organ failure may be associated with graft versus host disease (GVHD), in which the donor tissue and/or donor organ contains immune cells that react against the host recipient. Accordingly, the isolated EVs or compositions described herein may treat, prevent, or reduce the negative effects of PGD and/or GVHD.

The assessment of viability of a donor tissue and/or donor organ following transplantation depends on the tissue and/or organ. For example, the viability of the lung can be assessed based on the level of oxygenation achieved by the recipient following transplantation. Accordingly, an organ-specific assessment can be compared with the clinically accepted criteria for said organ-specific assessment. For example, oxygenation is an accepted standard for assessing lung function, so can be analysed in a recipient following lung transplantation and compared with relevant population data for the expected oxygenation for the subject, or in comparison to oxygenation achievable by the recipient prior to transplantation. Techniques for assessing organ function are known to the skilled person. In some embodiments, the assessment of viability may be characterised by improved organ graft function in the long term (e.g. 1, 2, 3, 4, 5, 6, 7, 8, 9, 10, 11 or 12 months post-transplantation) compared with organ function within one week following transplantation of the subject, or to a control subject having undergone a tissue and/or organ transplant and experiencing delayed graft function who has not been exposed to isolated EVs. Alternatively, or additionally, the control for comparison may be a tissue and/or organ that has not been exposed to isolated EVs. Viability may also be referred to as preservation.

In some embodiments, the isolated EVs and/or compositions described herein have been introduced to an ex-vivo donor tissue and/or ex-vivo donor organ before and/or are introduced during transplantation to the recipient. For example, the isolated EVs and/or compositions described herein may have been introduced to an ex-vivo donor tissue and/or ex-vivo donor organ during perfusion of the donated tissue and/or organ. In lung transplantation, the isolated EVs and/or compositions described herein may have been introduced to an ex-vivo donor tissue and/or ex-vivo donor organ during EVLP.

Alternatively, or additionally, the isolated EVs and/or compositions described herein may be introduced to the ex-vivo donor tissue and/or ex-vivo donor organ at the time of transplantation and/or at an interval of time following completion of transplantation. For example, the isolated EVs and/or compositions described herein may be introduced to the ex-vivo donor tissue and/or ex-vivo donor organ 1 hour following transplantation. As a further example, the isolated EVs and/or compositions described herein may be introduced to the ex-vivo donor tissue and/or ex-vivo donor organ 12 hours following transplantation. As a further example, the isolated EVs and/or compositions described herein may be introduced to the ex-vivo donor tissue and/or ex-vivo donor organ 1 hour and 12 hours following transplantation. Each of these examples may be in addition to or replacement of the isolated EVs and/or compositions described herein being introduced at the time of transplantation. A subsequent administration of isolated EVs and/or compositions described herein may be to ‘top-up’ the levels of EVs or activity thereof. For example, a serum or biopsy sample from the donated tissue or organ may reveal that the concentration of an inflammatory cytokine has recovered from the EV-dependent reduction in its expression, which may be used to assess whether the recipient needs a top-up of EVs. Accordingly, subsequent administrations of isolated EVs and/or compositions described here may be in a subject in need thereof.

In some embodiments, the isolated EVs or composition comprising isolated EVs is administered in combination with a further agent, sequentially, simultaneously and/or subsequently. For example, the further agent may be administered as part of the composition comprising isolated EVs. In some embodiments, the further agent is selected from the group consisting of anti-inflammatory agents, immunosuppressive agents, anti-rejection agents/drugs (e.g. prednisone, tacrolimus, etc) and any combinations thereof. The term “anti-inflammatory agent” indicates that the agent or drug reduces or prevent an immune response that causes inflammation. The term “immunosuppressive agents” indicates that the agent or drug blocks or reduces the activity of an immune response, which may be a proinflammatory or anti-inflammatory response. By “anti-rejection composition” we include the term “anti-rejection drug”. This term is commonly used in the art to refer to immunosuppressants, particularly those used to treat, prevent and/or reduce transplant rejection. Therefore, the term “anti-rejection composition” includes the meaning of an immunosuppressant that prevents and/or reduces pathologies associated with transplant rejection. The isolated EVs and compositions described herein may be used to replace or supplement (i.e. used in combination) other anti-rejection drugs that have failed to treat, prevent, and/or reduce transplant rejection. An agent or drug may fall within the definition of any one or more of these terms, and so the terms may be used herein interchangeably.

In some embodiments, the isolated EVs or composition comprising isolated EVs are administered more than once. For example, administration may occur 2, 3, 4, 5, 6, 7, 8, 9, 10 or more times.

As used herein, the term “administering” or “administration”, refers to the placement of isolated EVs or a composition as disclosed herein into a subject by a method or route which results in at least partial localisation of the agents or composition at a desired site. “Route of administration” may refer to any administration pathway known in the art, including but not limited to oral, topical, aerosol, nasal, via inhalation, anal, intra-anal, peri-anal, transmucosal, transdermal, parenteral, enteral, or local. “Parenteral” refers to a route of administration that is generally associated with injection, including intratumoral, intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous, intraarterial, subarachnoid, subcapsular, subcutaneous, transmucosal, or transtracheal. Via the parenteral route, the agent or composition may be in the form of solutions or suspensions for infusion or for injection. Via the enteral route, the agent or composition can be in the form of capsules, gel capsules, syrups, suspensions, solutions, emulsions, or lipid vesicles or polymer vesicles allowing controlled release. Via the topical route, the agent or composition can be in the form of aerosol, lotion, cream, gel, ointment, suspensions, solutions or emulsions.

In some embodiments, the isolated EVs or composition comprising isolated EVs are administered intravenously, intraarterially, intravascularly and/or intrabronchially. In a preferred embodiment, the EVs are administered intravenously. The site of intravenous administration is preferably upstream of the site at which a pathology exists, for example upstream of an inflamed tissue or a transplantation site.

It will be appreciated that administration may be before, during and/or after transplantation is performed. For example, administration before transplantation may be intravenous (IV) to the donor before the tissue or organ has been removed from the donor, either directly into the tissue or organ of interest and/or into the blood stream of the donor, preferably wherein the administration is directly into the tissue or organ of interest. Alternatively, or additionally, administration may be directly into the donor tissue or donor organ after it has been removed from the donor, and/or by submerging the donor tissue or donor organ into a conditioning media during transportation. For example, in EVLP, administration may be via IV administration directly into the donor tissue or donor organ (e.g. donor lung or donor lung tissue). In a particularly preferred embodiment, the IV administration is directly into the donor tissue or donor organ after its removal from the donor.

In some embodiments, the organ is subjected to an effective amount of isolated EVs about 30-36 hours, about 25-30 hours, about 20-25 hours, about 15-20 hours, about 10-15 hours, about 5-10 hours, about 1-5 hour or combinations thereof, prior to implantation of the organ in the subject. In some embodiments, the organ is treated with an effective amount of isolated EVs about 1 hour, 2 hours, 3 hours, 4 hours, 5 hours, 6 hours, 7 hours, 8 hours, 9 hours, 10 hours, 10-15 hours, 15-20 hours, 20-24 hours or combinations thereof, prior to implantation of the organ in the subject.

Administration during transplantation may be IV administration into the donor tissue or donor organ upon its transplantation into the tissue or organ recipient. In this context, “during” includes at any point during which a surgeon considers the transplantation process to be ongoing. For example, administration may be prior to the donor tissue or donor organ being inserted into a recipient but after the donor tissue or donor organ has been removed from a perfusion system or storage container. As a further example, administration may be simultaneous to the donor tissue or donor organ being grafted to a recipient or immediately after engraftment. Alternatively, or additionally, administration during transplantation may be IV administration into the bloodstream of the recipient while they are undergoing a transplantation procedure.

Administration after transplantation may be IV administration directly into the donor tissue or donor organ that has been grafted into the recipient, following a transplantation procedure. Alternatively, or additionally, administration after transplantation may be IV administration into the bloodstream of the recipient at any time following termination of a transplantation procedure. For example, this may be a continuation of the administration to the bloodstream that occurs during the transplantation procedure, immediately after the transplantation procedure, or hours after the transplantation procedure. In some embodiments, administration after transplantation may be at least 1 hour after transplantation, for example at least 2, 3, 4, 5, 6, 7, 8, 9, 10, 11, 12, 13, 14, 15, 16, 17, 18, 19, 20, 21, 22, 23 and/or 24 hours after transplantation. In a preferred embodiment, the administration may be 1 hour and 12 hours after transplantation. In various embodiments, the administration is up to any one or more of one month, two months, six months, twelve months, 18 months, 24 months or 30 months after transplant.

In some embodiments, delayed graft function (DGF) is observed in the subject that has undergone organ (e.g. skin) transplant. Known clinical interventions may be needed in the case of DGF, which may vary depending on the organ, e.g. dialysis may be needed in the subject within seven days of transplant for a kidney. In various embodiments, a reduction in the need for the intervention is observed about 2 weeks, 3 weeks or 4 weeks after the transplant. In further embodiments, the reduction in the need for the intervention is observed about 2-4 weeks, 1-3 months, 3-6 months, 6-9 months, 9-12 months or 12-15 months after the transplant.

In situations where administration occurs multiple times, any of the readouts described herein may be with respect to any of the administrations.

Graft dysfunction as described herein may be selected from the group consisting of primary graft dysfunction (PGD), cardiac allograft rejection and cardiac allograft vasculopathy.

In some embodiments, the use or method of treatment may be for a condition that occurs downstream of graft dysfunction and/or GVHD. Accordingly, by preventing and/or treating the upstream condition, one provides a use or method that prevents and/or treats the downstream condition.

The assessment of viability of a donor tissue and/or donor organ following transplantation depends on the tissue and/or organ. For example, the viability of the lung can be assessed based on the level of oxygenation achieved by the recipient following transplantation. Accordingly, an organ-specific assessment can be compared with the clinically accepted criteria for said organ-specific assessment. For example, oxygenation is an accepted standard for assessing lung function, so can be analysed in a recipient following lung transplantation and compared with relevant population data for the expected oxygenation for the subject, or in comparison to oxygenation achievable by the recipient prior to transplantation. Techniques for assessing organ function are known to the skilled person. In some embodiments, the assessment of viability may be characterised by improved organ graft function in the long term (e.g. 1, 2, 3, 4, 5, 6, 7, 8, 9, 10, 11 or 12 months post-transplantation) compared with organ function within one week following transplantation of the subject, or to a control subject having undergone a tissue and/or organ transplant and experiencing delayed graft function who has not been exposed to isolated EVs of the present invention. Alternatively, or additionally, the control for comparison may be a tissue and/or organ that has not been exposed to isolated EVs. Viability may also be referred to as preservation. The control for comparison may also be a tissue and/or organ that has been exposed to isolated EVs from a source other than TAF MSCs, for example ASC-EVs.

By “conditioning media” we refer to a media comprising isolated EVs that is suitable for conditioning a donor tissue and/or donor organ, optionally wherein the media is decellularised. A conditioning media may be used in a donor prior to removal of a tissue and/or organ, in a separate vessel in which the donor tissue and/or donor organ is stored (e.g. an EVLP chamber), or both. By “conditioning” we include the meaning that the media acts upon a tissue and/or organ in a way that retains, restores and/or rejuvenates the tissue and/or organ to a state closer to being physiologically healthy. Alternatively, or additionally, “conditioning” may refer to the retention, restoration and/or rejuvenation of a tissue and/or organ to parameters that would pass a criterion for said tissue and/or organ being deemed suitable for transplantation. Transplantation criteria for a tissue and/or organ, which varies depending on the tissue and/or organ, are known to the skilled person.

By “perfusion fluid” we refer to a fluid that is suitable for use during perfusion. Types of perfusion fluid are known in the art and vary depending on the perfusion technique, i.e. the perfusion fluid may be one that is suitable for use in perfusing a specific tissue and/or organ. Accordingly, the perfusion fluid can be any known perfusion fluid for use in perfusing a tissue and/or organ of interest, wherein the perfusion fluid further comprising isolated EVs. Use of isolated EVs in perfusion fluid may be in addition to or replacement of isolated EVs being present in a preceding and/or foregoing conditioning media. In some embodiments, the perfusion fluid is comprised of the same components as the conditioning media. In some embodiments, the perfusion fluid is comprised of different components as the conditioning media. Preferably, the isolated EVs used in the perfusion fluid are the same as those used in the conditioning media.

By “injection fluid” we refer to a fluid that is suitable for being injected into a tissue and/or organ. The injection fluid may be for use prior to, during and/or after transplantation of a donor tissue and/or donor organ. Use of isolated EVs in injection fluid may be in addition to or replacement of isolated EVs being present in a preceding and/or foregoing conditioning media and/or perfusion fluid. In some embodiments, the injection fluid is comprised of the same components as the conditioning media. In some embodiments, the injection fluid is comprised of different components as the conditioning media. In some embodiments, the injection fluid is comprised of the same components as the perfusion fluid. In some embodiments, the injection fluid is comprised of different components as the perfusion fluid. Preferably, the isolated EVs used in the injection fluid are the same as those used in the conditioning media and/or perfusion fluid.

Accordingly, the terms “conditioning media”, “perfusion fluid”, and “injection fluid” are used herein interchangeably. Therefore, any component referred to with respect to one of these terms is equally applicable for inclusion in a composition referred to by another of these terms.

In some embodiments, the conditioning media further comprises at least one antibiotic, vitamin, prostaglandin, bicarbonate and/or anticoagulant (e.g. heparin).

In some embodiments, the perfusion fluid further comprises at least one antibiotic, vitamin, prostaglandin, bicarbonate and/or anticoagulant (e.g. heparin).

In some embodiments, the injection fluid further comprises at least one antibiotic, vitamin, prostaglandin, bicarbonate and/or anticoagulant (e.g. heparin).

In some embodiments, the isolated EVs or decellularised composition are/is incorporated into a device, such as a medical device. The device may be selected from the group consisting of a hydrogel, a dressing, a bandage, a suture, and a plaster. In some embodiments, the devices comprise the isolated EVs or decellularised composition. In some embodiments, the devices are impregnated with the isolated EVs or decellularised composition. The devices comprising or impregnated with the isolated EVs or decellularised composition incorporate such active agents in a way that allows them to be liberated from the device. For example, a hydrogel impregnated with the isolated EVs or decellularised composition may liberate the isolated EVs or decellularised composition when placed over a wound.

As used herein, the terms “treat”, “treatment”, “treating”, or “amelioration” when used in reference to a disease, disorder or medical condition, refer to both therapeutic treatment and prophylactic or preventative measures, wherein the object is to prevent, reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results or lower the chances of the individual developing the condition even if the treatment is ultimately unsuccessful. Those in need of treatment include those already with the condition as well as those prone to have the condition or those in whom the condition is to be prevented.

“Beneficial results” or “desired results” may include, but are in no way limited to, lessening or alleviating the severity of the disease condition, preventing the disease condition from worsening, curing the disease condition, preventing the disease condition from developing, lowering the chances of a patient developing the disease condition, decreasing morbidity and mortality, and prolonging a patient's life or life expectancy. As non-limiting examples, “beneficial results” or “desired results” may be alleviation of one or more symptom(s), diminishment of extent of the deficit, stabilised (i.e., not worsening) state of allograft function (e.g. skin allograft), delay or slowing of organ function, and amelioration or palliation of symptoms associated with end stage organ disease.

A donor or recipient may be referred to as a subject. As used herein, a “subject” means a human or animal. Usually, the animal is a vertebrate such as a primate, rodent, domestic animal, or game animal. Primates include chimpanzees, cynomologous monkeys, spider monkeys, and macaques, e.g., Rhesus. Rodents include mice, rats, woodchucks, ferrets, rabbits and hamsters. Domestic and game animals include pigs, cows, horses, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, pig, mouse, rat, dog, cat, horse, or cow, but are not limited to these examples. In an embodiment, the subject is human. In addition, the methods described herein can be used to treat domesticated animals and/or pets.

A subject can be one who has been previously diagnosed with or identified as suffering from or having a condition in need of treatment (e.g., skin wounds or inflammation, such as dermal inflammation) or one or more complications related to the condition, and optionally, have already undergone treatment for the condition or the one or more complications related to the condition. Alternatively, a subject can also be one who has not been previously diagnosed as having a condition or one or more complications related to the condition. For example, a subject can be one who exhibits one or more risk factors for a condition, or one or more complications related to the condition or a subject who does not exhibit risk factors. For example, a subject can be one who exhibits one or more symptoms for a condition, or one or more complications related to the condition or a subject who does not exhibit symptoms. A “subject in need” of diagnosis or treatment for a particular condition can be a subject suspected of having that condition, diagnosed as having that condition, already treated or being treated for that condition, not treated for that condition, or at risk of developing that condition.

A therapeutically or prophylactically significant reduction in a symptom is, e.g., at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject or the state of the subject prior to administering isolated EVs. Measured or measurable parameters include clinically detectable markers of disease, for example, elevated or depressed levels of a biological marker, as well as parameters related to a clinically accepted scale of symptoms or markers for fibrosis and/or inflammation. It will be understood, however, that the total daily usage of the compositions and formulations as disclosed herein will be decided by the attending physician within the scope of sound medical judgment. The exact amount required will vary depending on factors such as the type of disease being treated, gender, age, and weight of the subject.

All of the features disclosed in this specification (including any accompanying exhibits, claims, abstract and drawings), and/or all of the steps of any method or process so disclosed, may be combined in any combination, except combinations where at least some of such features and/or steps are mutually exclusive. The disclosure is not restricted to the details of any foregoing examples. The disclosure extends to any novel one, or any novel combination, of the features disclosed in this specification (including any accompanying claims, abstract and drawings), or to any novel one, or any novel combination, of the steps of any method or process so disclosed.

Those skilled in the art will appreciate that in some examples, the actual steps taken in the processes illustrated or disclosed may differ from those shown in the figures. Depending on the example, certain of the steps described above may be removed, others may be added. For example, the actual steps or order of steps taken in the disclosed processes may differ from those shown in the figure. Depending on the example, certain of the steps described above may be removed, others may be added. Furthermore, the features and attributes of the specific examples disclosed above may be combined in different ways to form additional examples, all of which fall within the scope of the present disclosure.

Conditional language, such as “can”, “could”, “might”, or “may”, unless specifically stated otherwise, or otherwise understood within the context as used, is generally intended to convey that certain examples include, while other examples do not include, certain features, elements, or steps. Thus, such conditional language is not generally intended to imply that features, elements, or steps are in any way required for one or more examples or that one or more examples necessarily include logic for deciding, with or without user input or prompting, whether these features, elements, or steps are included or are to be performed in any particular example. The terms “comprising”, “including”, “having”, and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Likewise, the term “and/or” in reference to a list of two or more items, covers all of the following interpretations of the word: any one of the items in the list, all of the items in the list, and any combination of the items in the list. Further, the term “each”, as used herein, in addition to having its ordinary meaning, can mean any subset of a set of elements to which the term “each” is applied. Additionally, the words “herein”, “above”, “below”, and words of similar import, when used in this application, refer to this application as a whole and not to any particular portions of this application.

Conjunctive language such as the phrase “at least one of X, Y, and Z”, unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately”, “about”, “generally”, and “substantially” as used herein represent a value, amount, or characteristic close to the stated value, amount, or characteristic that still performs a desired function or achieves a desired result. For example, the terms “approximately”, “about”, “generally”, and “substantially” may refer to an amount that is within less than 10% of, within less than 5% of, within less than 1% of, within less than 0.1% of, and within less than 0.01% of the stated amount. As another example, in certain examples, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

Various modifications to the implementations described in this disclosure may be readily apparent to those skilled in the art, and the generic principles defined herein may be applied to other implementations without departing from the spirit or scope of this disclosure. Thus, the disclosure is not intended to be limited to the implementations shown herein, but is to be accorded the widest scope consistent with the principles and features disclosed herein. Certain examples of the disclosure are encompassed in the claim set listed below or presented in the future.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The listing or discussion of an apparently prior published document in this specification should not necessarily be taken as an acknowledgement that the document is part of the state of the art or is common general knowledge. Preferences, options and embodiments for a given aspect, feature or parameter of the invention should, unless the context indicates otherwise, be regarded as having been disclosed in combination with any and all preferences, options and embodiments for all other aspects, features and parameters of the invention. Embodiments and features of the present invention are also outlined in the following items and illustrated by the following non-limiting examples.

### EXAMPLES

### Example 1—Extracellular Vesicle Production in 2D Starvation

### Materials and Methods

### Materials

Accutase (StemPro, 1110501).
MSC-Brew GMP Basal medium without phenol red (170-076-315) (Miltenyi Biotec).
MSC-Brew GMP Supplement 1 (170-076-323) (Miltenyi Biotec).
MSC-Brew GMP Supplement 2 (170-076-320) (Miltenyi Biotec).
T150 CellBIND (Corning).
CD10-sorted TAF042 p7 Batch 1 (Study 260).

### Methods

### Notes

A good volume of supernatant to initiate characterization is 10-30 ml, which means a TTS or T175 flask is preferred.

Note that “Collection medium” refers to normal culture medium without particles, meaning it is without serum/platelet lysate or with particle-deprived serum/platelet lysate.

### Protocol

1. Culture the cells as usual in three distinct flasks/dishes until they achieve 70% confluence.

2. Wash the cells 2 times with PBS to remove traces of serum.

3. Introduce collection medium and directly collect 1 ml of supernatant from each flask. Spin the collected media at 2,000×g during 10 min to remove any debris and store the supernatant at −80° C. (sample=Flask X, t=0).

4. After 24 h, collect the conditioned medium from one flask, spin it at 2,000×g during 10 min and store the CM at −80° C. (sample=StaNation 24 h). Detach the cells from the surface then count them using automatic cell counting or microscopy manual counting. Write the number of cells and the viability.

5. Repeat step 4 at 48 h and 72 h with one flask at each timepoint.

An exemplary experimental setup is demonstrated in Table B.

TABLE B
Experimental setup.

| Day | Action |
|---|---|
| 1 | Seeding 2500 cells/cm2 in T 150 × 3 in 30 mL complete |
| | GMP-brew. |
| 4 | Medium change. |
| 5 | Expected 70% confluency. T 0. 30 mL. GMP-brew without media |
| | supplements. |
| 6 | T 24 |
| 7 | T 48 |
| 8 | T 72 |
| 11 | Shipment for analysis. |

### Collection of Medium

T0: 1 mL from each of the three flasks.
T24: 1 mL×5+24 mL. Count cells. Check viability.
T48: 1 mL×5+24 mL. Count cells. Check viability.
T72: 1 mL×5+24 mL. Count cells. Check viability.

### Thawing Protocol

Thawing is considered a stressful and dangerous step for cells. This protocol is aimed to ensure that a high proportion of the frozen cells survive the thawing procedure.

Reagents and cell culture requirements: Burker chamber, cell culture flasks, Centrifuge (Ninolab Sorvall ST4 Plus REF75009909, Ser. No. 42/706,558), Falcon tubes (15 ml), Falcon tubes (SO ml), Cell culture medium of choice, Trypan Blue (SigmaAldrich, T8154), dry ice.

This procedure can be used for cells frozen with DMSO solution (10% DMSO in FBS) and CryoStor.

1. Collect cryovials on dry ice.

2. Perform the thawing steps quickly (<1 minute) according to step 4 and 5.

3. Pre-warm sterile water or EtOH in a 50 ml tube to 37° C. in the a heating bath.

4. Pre-warm the culture medium to 37° C. in the heating bath.

5. Place 2/3 of the frozen cryovial (not the content) into the water of a 50 ml conical tube (keeping the lid dry).

6. Swirl cryovial until a single mm-large crystal is the only ice remaining in the tube.

7. Add 1 mL of appropriate medium dropwise slowly to the pellet and mix gently.

8. Transfer the cell suspension to a 15 ml tube.

9. Wash the cryovial with 1 mL extra medium and transfer to the 15 mL tube.

10. Add approximately 12 mL warm medium dropwise to the cell suspension during several minutes to let the DMSO diffuse slowly out of the cells. This slow diffusion is better for cell survival.

11. Centrifuge at 300×g for 5 minutes at RT.

12. Aspirate the supernatant and avoid touching the pellet.

13. Determine cell number and viability.

14. Seed the cells in the defined density.

15. Culture the cells at 37° C. in the incubator with 5% CO2 and >95% humidity.

TAF Cell Splitting and Culture Procedure after Initial Seeding

The aim of this protocol is to provide the best conditions to obtain the maximum amount of high-quality mesenchymal stem cells (MSCs) from term amniotic fluid (TAF) samples. High quality is understood as propagation capacity and differentiation capacity of MSCs.

Reagents and cell culture requirements: Burker chambers, Medium 3 (see below), GMP-MSC-brew (see below), T25, TISO, or T75 CellBIND® flasks (Corning), Trypan blue exclusion (Sigma-Aldrich, T8154), Accutase (StemPro, 1110501), 2, 5, 10, 25, 50 ml pipettes, 50 ml tubes, 15 ml tubes, Eppendorf tubes, pipette tips 20, 300, 1000 μL, pipettes, sterile 500 ml filter flasks (431097, Corning).

Medium 3: StemMACS MSC expansion XF (Miltenyi Biotec) (130-104-182) with Supplement. Xenofree and serum free. Supplement (7 ml) is thawed in the incubator and aliquoted into 700-750 UL and refrozen. Date is written on the flask when opened. Medium without supplement is used within one month. Thaw an aliquot of supplement and add 700 μL/50 ml M3-medium to obtain complete medium, and protect from light. Use the complete medium within one week.

MSC-brew GMP Basal medium+ supplements: GMP-BrewStemMACS MSC expansion XF (Miltenyi Biotec) (170-076-322) with Supplements I (170-076-323) and II (170-076-320) is xeno-free, serum-free and GMP-approved. Supplements (5 ml) are thawed in the incubator and aliquoted into 500 UL and refrozen. Date is written on the flask when opened. From a 2 L GMP-brew bag, cut the hose, use 4 500 ml-filter flasks, and move all media over to the flasks. Medium without supplement (basal) is used within one month. Thaw aliquots of supplement I and II and add 500 μL of each/50 ml basal medium to obtain complete medium. Alternatively, if a lot of media is used, add 5 ml of each supplement to a full bottle of 500 ml basal brew and use within two weeks. Protect from light. Complete medium is used within two weeks.

### Methodology:

1. Cell seeding densities (SD)/cm². Day of medium change (MC) and day of harvest.

| | | | |
|---|---|---|---|
| Day 0 | SD 1000 | SD 2500 | SD 5000 |
| Day 1 | | | |
| Day 2 | | | MC |
| Day 3 | MC | MC | Harvest |
| Day 4 | | Harvest | |
| Day 5 | Harvest | | |

2. Cell monitoring:
a. The cells have to be checked under the microscope with low and high magnification one day after seeding and before each medium change and cell dissociation.
b. Check whether there is the presence of debris and/or contamination.

3. Media change:
a. The medium is changed every 2nd/3rd day (see Step 1 table).
b. Remove all the medium with the vacuum pump.
c. Add x ml of cold medium to the flask. (5 ml to T-25, 15 ml to T-75, 30 ml to T-150). Cold medium is used by the production team, so should be used by R&D as well to have equal growth conditions for cells.
d. Close the lid of the flask tightly (make sure it is a filter flask).
e. Keep the flask in the incubator with hypoxia 4% O2 and 5% CO2.

4. Cell dissociation step:
a. Pre-warm M3 or GMP-brew in the bead warming basin.
b. Remove the medium by aspiration with the vacuum pump.
c. Add 700 L Accutase to each T25 flask, 2 ml to T75, 4 ml to T150, and keep them in the incubator for 3 minutes.
d. Check the detachment status of the cells after 3 minutes.
e. When cells start to round up, gently tap the flask for them to loosen.
f. Transfer the cell suspension to a tube.
g. Add the same volume of M3 or GMP-brew as Accutase to the flask to wash all cells off.
h. Add the medium with the remaining cells to the same tube.
i. Check that no cells remain attached to the flask.
j. When counting cells, consider approximately 1 ml medium per expected 1 million cells. An expected harvesting density is 40,000-70,000 cells/cm² and 80,000 cells/cm² is considered over-confluent.
k. Count at least three A-squares and at least 100 cells. Count cells that attach to two sides in the square and do not count the other two sides.

5. After the counting, 1000 to 5000 cells/cm² are seeded in the flasks and they are labelled as follows: name of the sample, medium, passage number, date, initials.

6. Any left-over cells can be frozen and stored for later analysis.

### Cell Counting Protocol

The aim of this protocol is to establish a standardized cell counting method.

Reagents and culture requirements: Burker chamber, Eppendorf tubes (1.5 mL), EtOH 70%, Falcon tube (15 mL), Hand tally counter, Trypan Blue exclusion (Sigma-Aldrich, T8154).

### Methodology:

The Burker chamber has 9 large squares (1 mm² each), divided by double lines (0.05 mm apart) into 16 group squares. The double lines form small 0.0025 mm² squares. The Chamber depth is 0.1 mm. All the 9 squares have a volume of 10-4 cm³ each, since 1 cm³=1 mL the result of the count represents the number of cells/0.1 μL.

To obtain the number of cells/mL, the total number of cells counted in all 9 squares should be divided by 9 and multiplied by 10,000, the dilution of the sample, and sample volume in mL.

However, most often not all 9 squares need to be counted. If there are enough cells, it will be sufficient to count at least 100 cells totally and at least three squares. As an example, 100 cells/A-square (one of the 9 squares) represents 1 million cells/mL in the corresponding sample if not diluted with trypan blue. If diluted 1:2 it represents 2 million cells/mL.

### Preparing the Hemocytometer:

1. Clean the glass of the hemocytometer and the coverslip with alcohol before use. Moisten the part of the coverslip that is not in contact with the counting area with alcohol and affix to the hemocytometer. The presence of Newton's refraction rings under the coverslip indicates proper adhesion.

### Preparing the Cell Suspension:

1. Dissociate cells, centrifuge, and suck off the supernatant.

2. Add an appropriate amount of medium to the pellet after centrifugation.

Add 1 mL medium/expected millions of cells approximately. This will give a counting of 100 cells/A-square if undiluted, and if diluted 50:50 with TB, 50 cells per A-square, which is appropriate to count.

3. Resuspend the pellet gently to ensure that cells are evenly distributed.

4. Before the cells have a chance to settle, take out one part e.g. 50 μL of cell suspension and place into the Eppendorf tube of 1.5 mL.

5. Add one part e.g. 50 μL of Trypan Blue exclusion into the Eppendorf tube of and mix carefully.

6. Take 10 UL of the stained cell suspension and let it get sucked into the Burker chamber. Make sure that the Newton rings are still there.

### Cell Counting:

1. Ensure that the microscope is sterile by spraying it with alcohol.

2. Focus the microscope on the grid lines of the hemocytometer with a 10× objective.

3. Using a hand tally counter, count the live, unstained cells (live cells do not take up Trypan Blue) in one set of 16 squares. When counting, employ a system whereby cells are only counted when they are set within a square or on the right-hand or bottom boundary line. Following the same guidelines, dead cells stained with Trypan Blue can also be counted for a viability estimation. Calculate a minimum of 100 cells.

4. Move the hemocytometer to the next set of 16 squares and carry on counting.

5. If cells are unevenly spread, prepare a new sample.

### Viability:

1. Calculate the mean cell count per A-square. Note: As long as an appropriate number of cells are present, it is sufficient with at least 100 cells totally and three squares.

2. Multiply by 10,000 (104).

3. Multiply by the dilution factor to correct (e.g. 2 for the 1:2 dilution) from the Trypan Blue addition.

4. This value is the number of viable cells/mL in the original cell suspension.

5. Multiply by the cell suspension volume (e.g. 3 for 3 mL).

6. The final value is the number of total viable cells in the original cell suspension.

### Results

On day 1, two vials of skin TAF MSC (sorted based on CD10 expression) p7 (passage 7) batch 1 were thawed and plated to p8 (passage 8). Considering an increasingly slower growth with higher passages, more cells were seeded than normal. Thus, 4600 cells/cm² were seeded. GMP cell culture medium MD57 #3 MA was used.

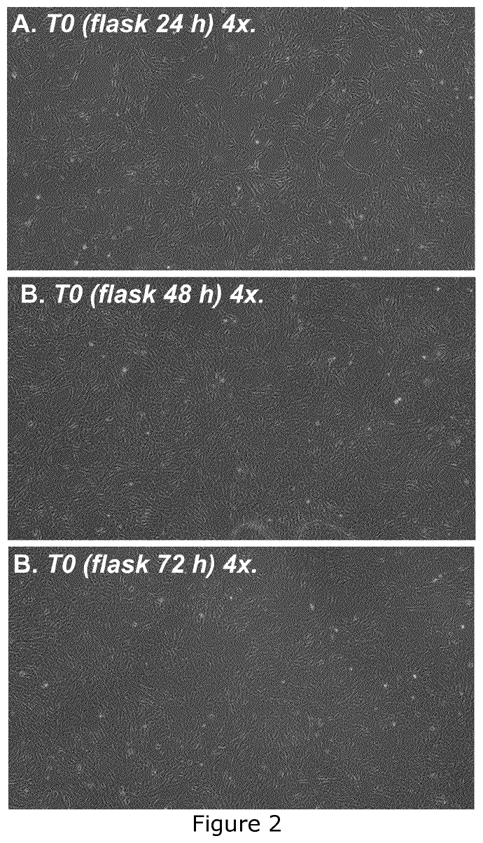

Instead of an expected medium change on day 4, the cells had grown very well (FIG. 2), so the starvation was initiated on the day 4 instead of the day 5.

PBS was used to wash cells twice, before 30 mL of GMP-brew without supplements was added to each flask. One sample per flask was immediately taken as T0, spun down for 10 min at 2000×g and the supernatants were frozen at −80° C. Pictures were taken of the three flasks (FIG. 2A-C).

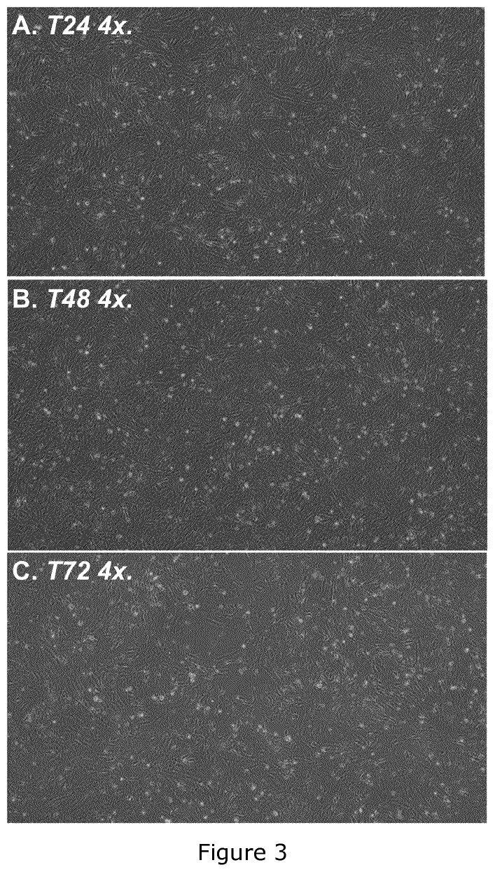

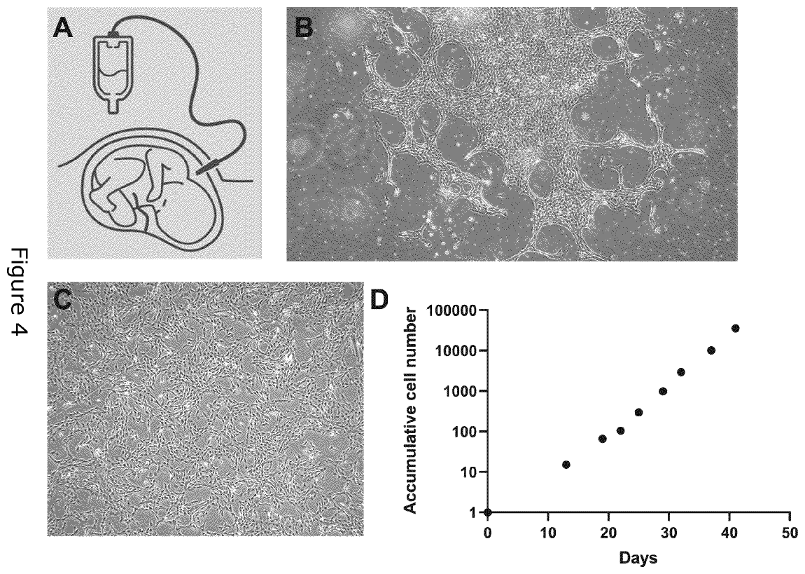

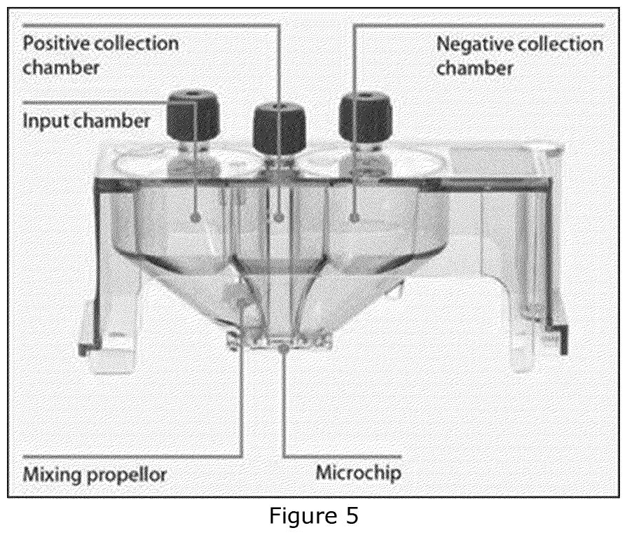

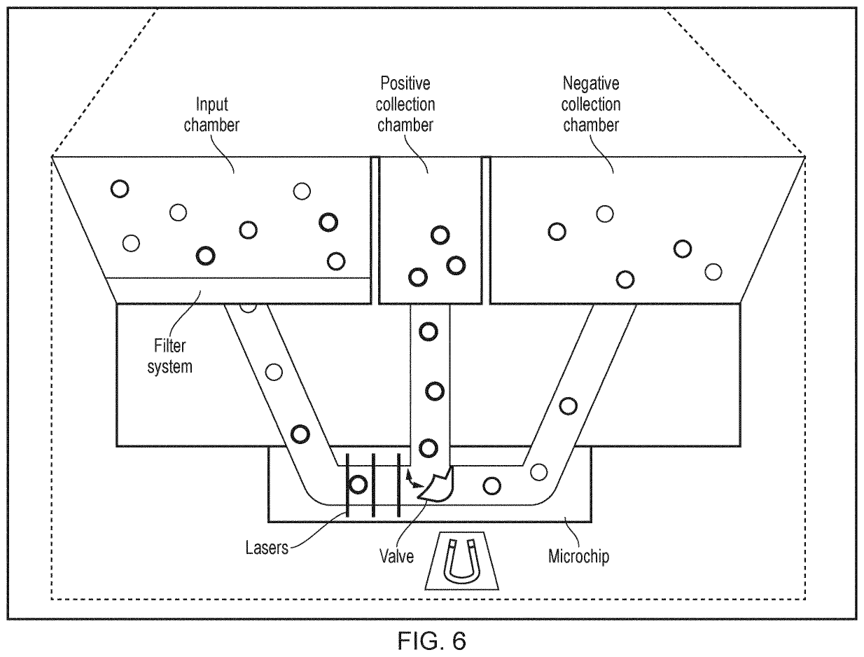

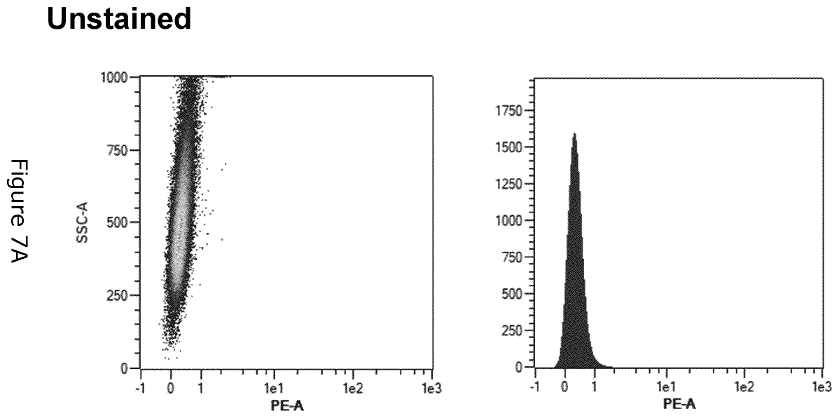

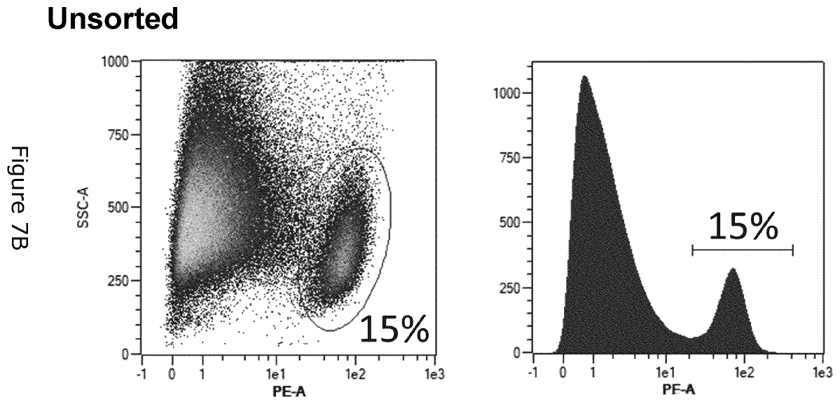

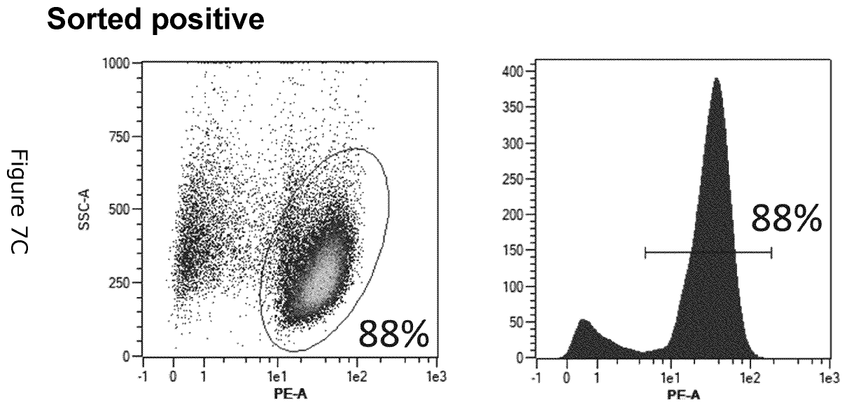

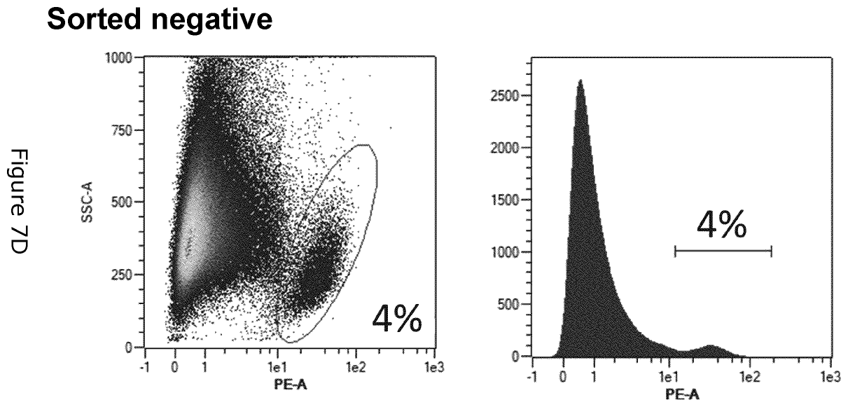

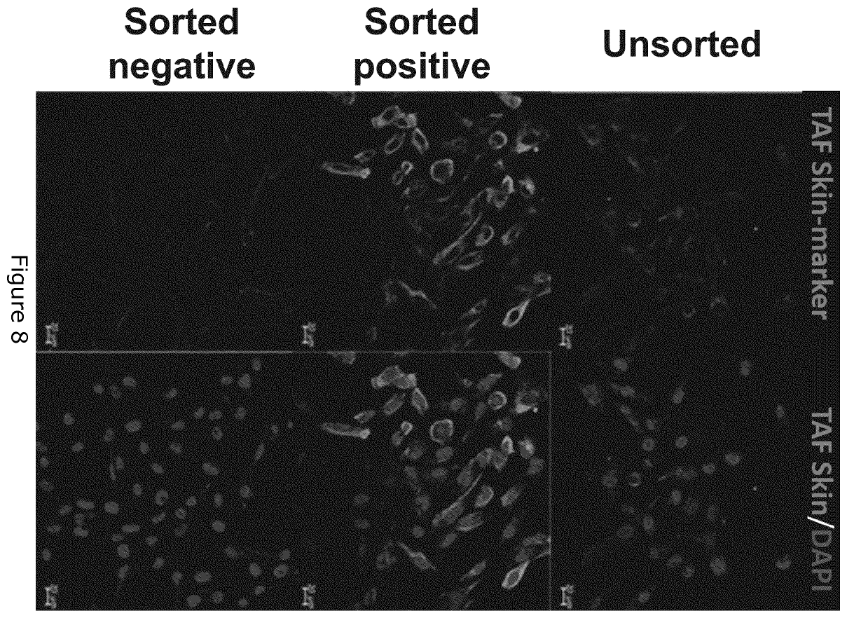

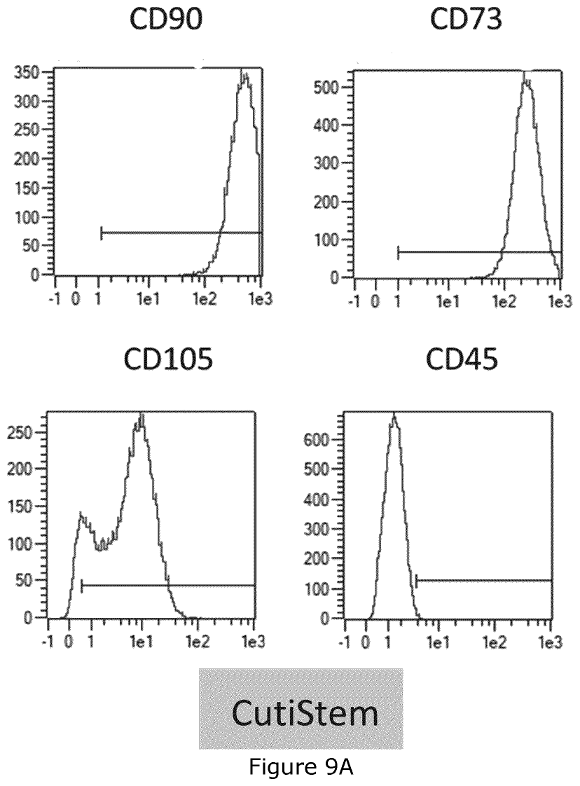

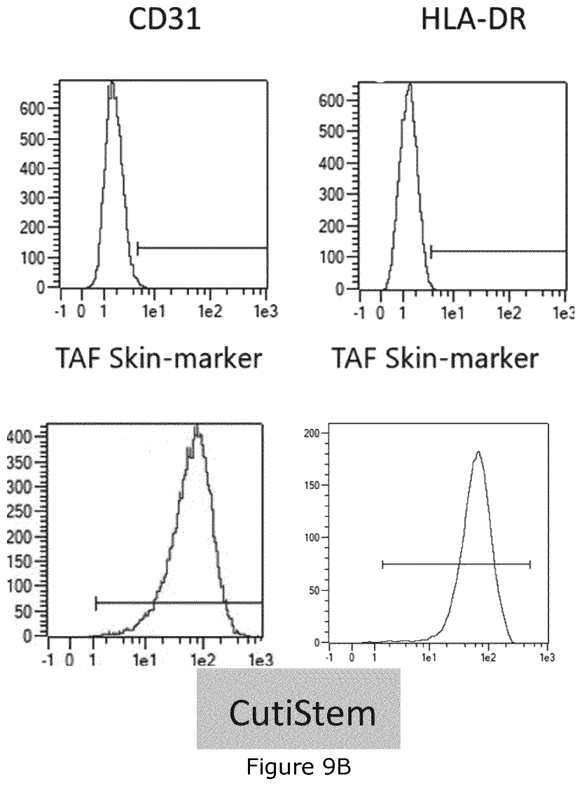

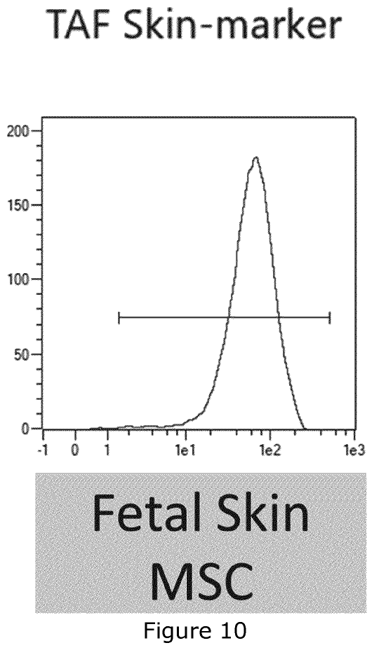

Samples were taken as described at T24, T48, and T72. Pictures were taken at each time point (FIG. 3A-C). Briefly, medium was collected in a 50 mL tube, centrifuged for 10 min at 2000×g and the supernatants were divided into 5×1 mL and 1×24 mL for all time points. Cells were counted at each time point (Table C). EV samples were directly frozen at −80° C. and later shipped to EVerZom on dry ice.

TABLE C
Cell count (Bürker) on time points 24, 48 and 72 h (M = million).

| Time point | Live cells | Dead cells |
|---|---|---|
| 24 h | 4.3M | 0.1M |
| 48 h | 2.3M | 0.2M |
| 72 h | 2.3M | 0.1M |

### Example 2—Characterisation of Extracellular Vesicles by ZetaView

### Materials and Methods

Skin specific TAF MSCs (i.e. TAF MSCs sorted based on expression of CD10) were used to generate these data, following the 2D EV production protocol of Example 1, with 3 T175 flasks containing 30 ml of media, and 3 times of culture (24 h/48 h/72 h).

Nanoparticle Tracking Analysis (NTA) measurements were taken, using ZetaView to assess size and concentration of EVs, and ExoView for tetraspanin measurement.

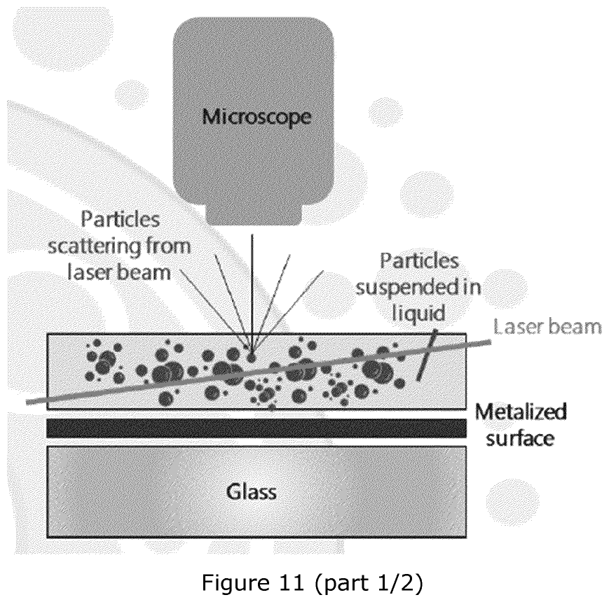

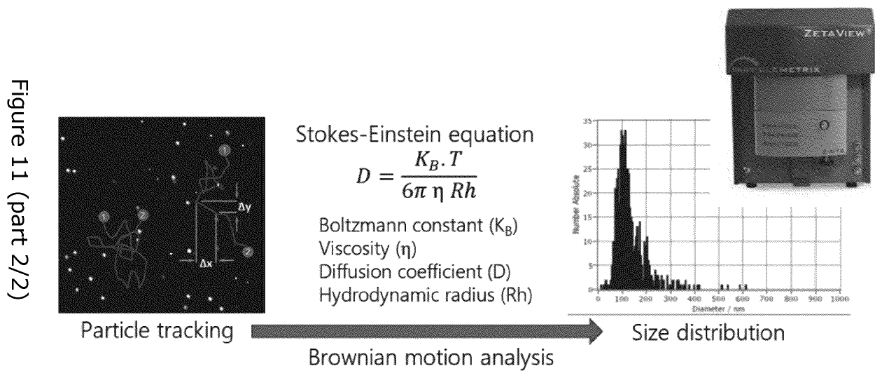

ZetaView (Particle Matrix™) with NTA technology is schematically represented in FIG. 11. NTA technology is based on Brownian particle motion. Individual particle tracking analysis uses particle-by-particle light scattering to provide size information (Stokes-Einstein equation). Each particle is analysed individually but simultaneously by measuring its scattering coefficient. In practice, it detects sizes between 70 and 1000 nm, corresponding to the hydrodynamic diameter of the particles. Concentration measurements are obtained from a direct count of the particles in the sample. Other standard techniques can be used to detect extracellular vesicles at sizes below 70 nm, such as electron microscopy.

### Results

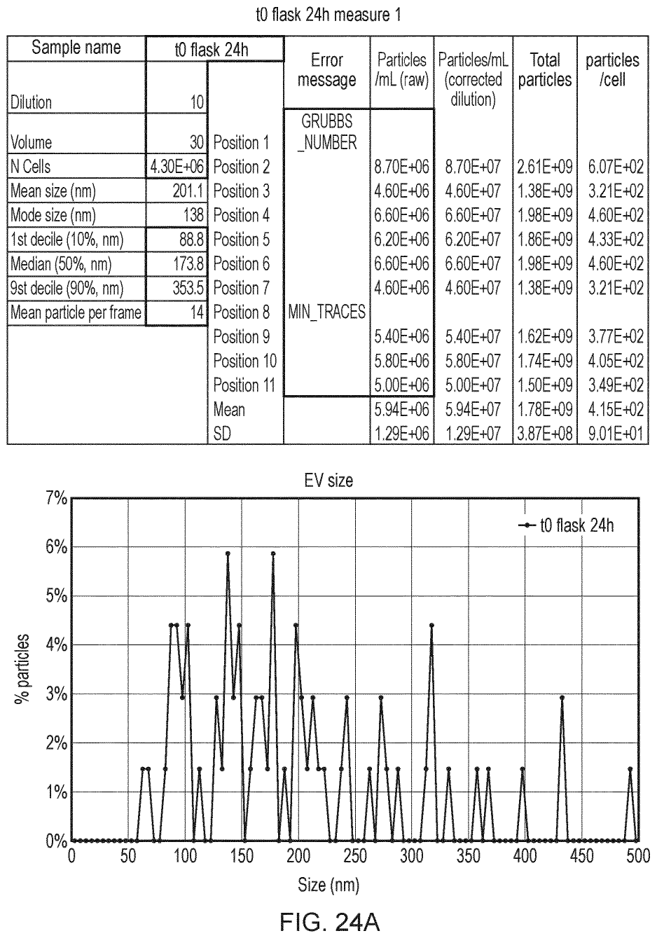

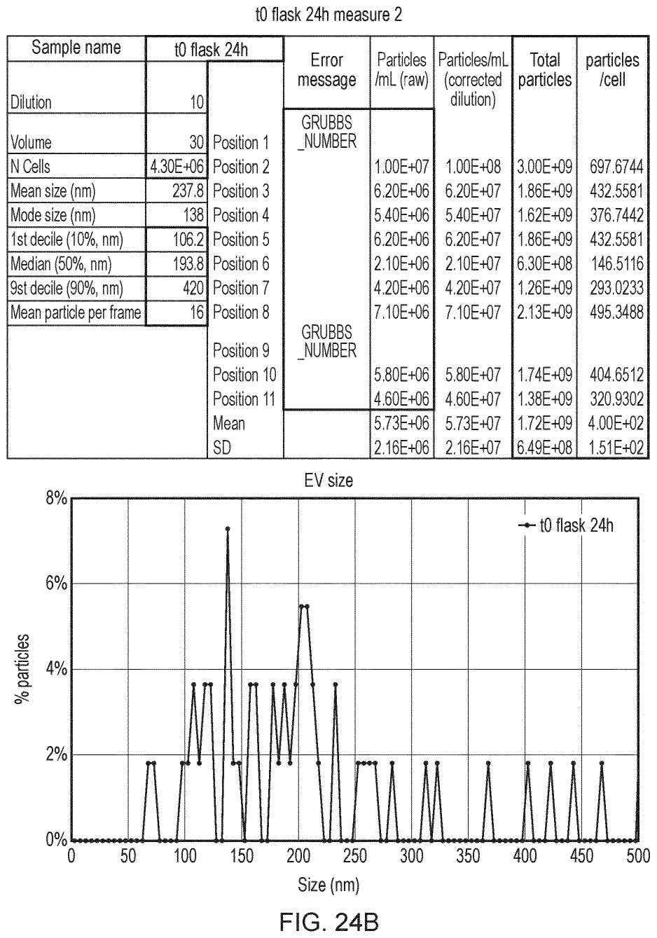

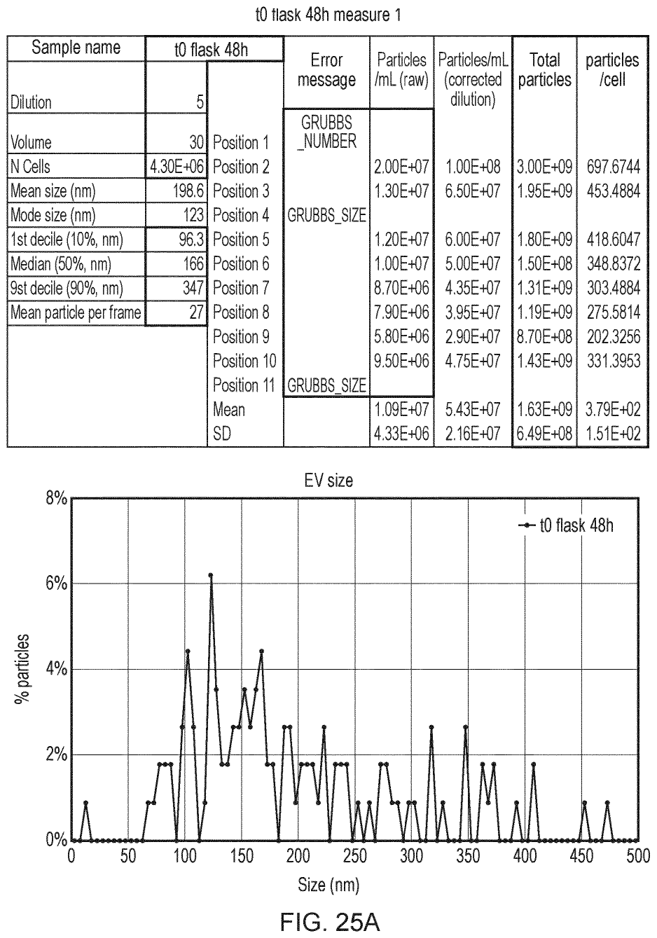

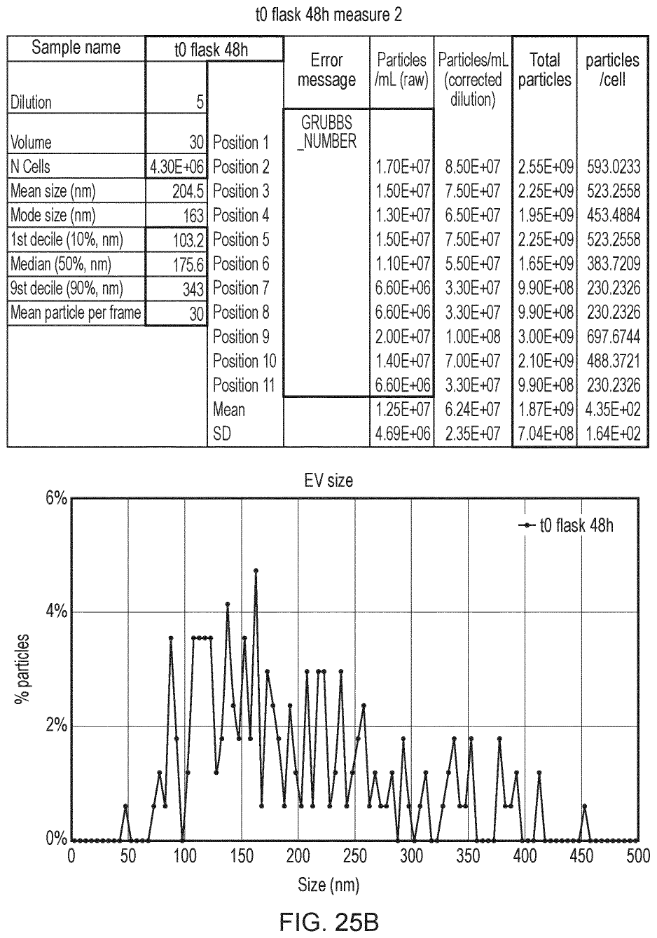

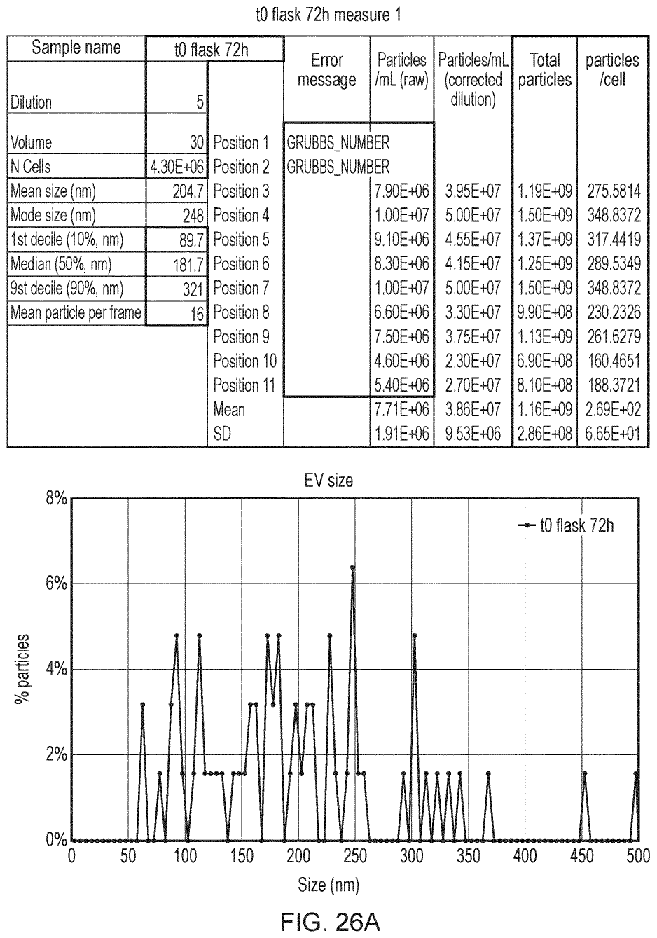

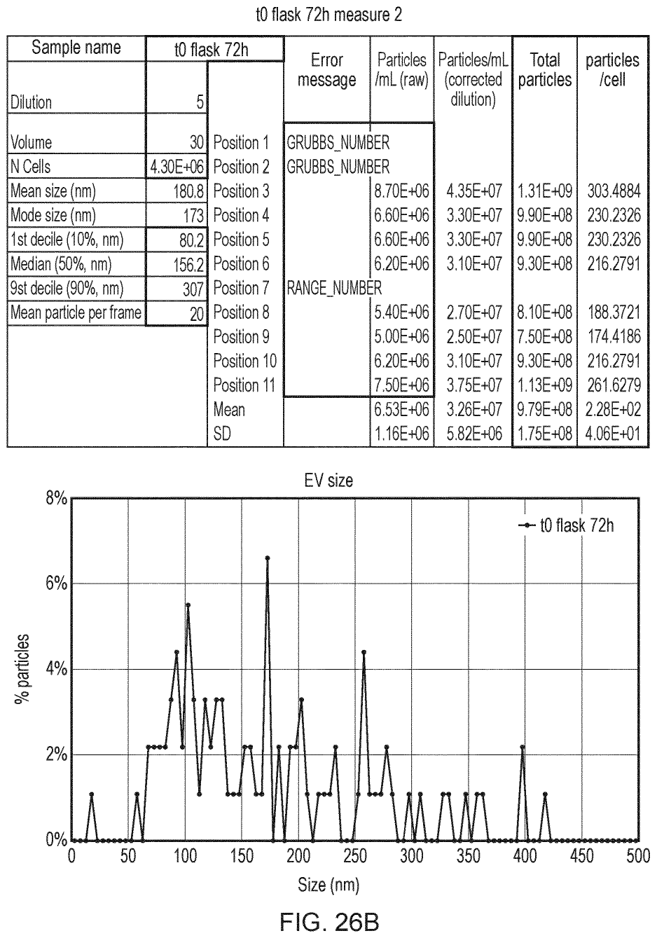

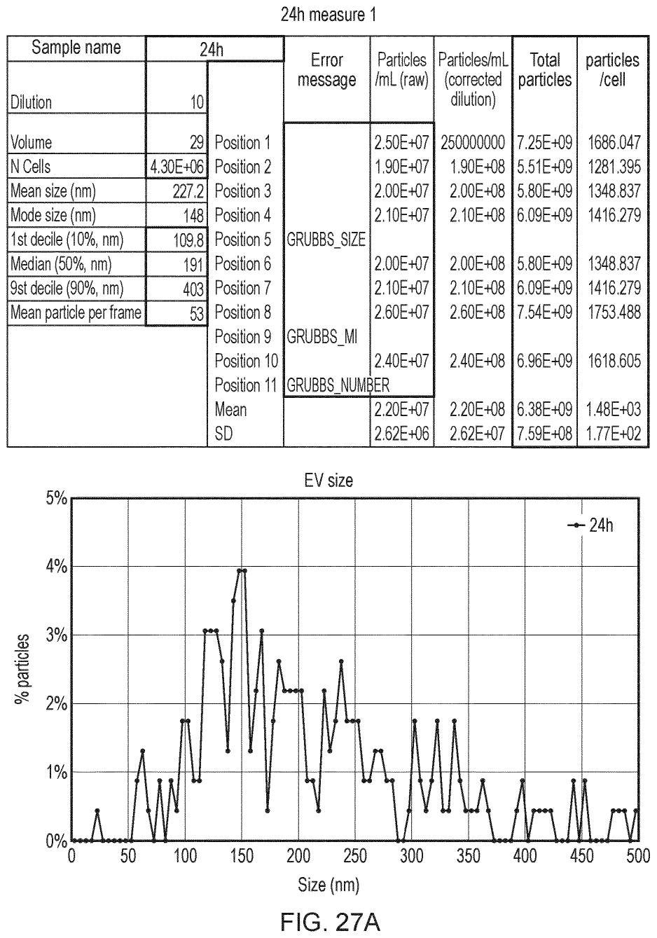

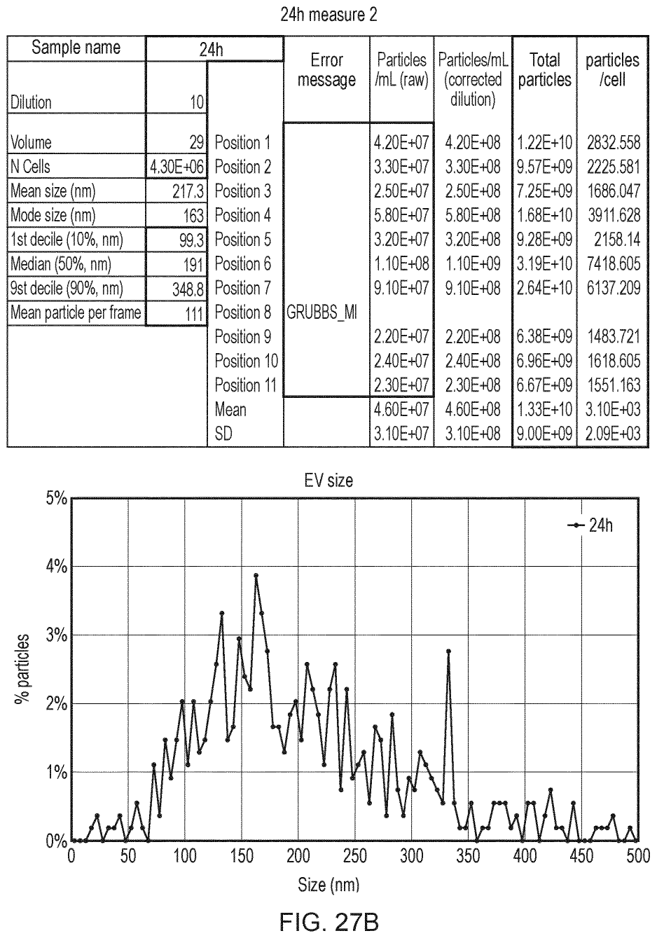

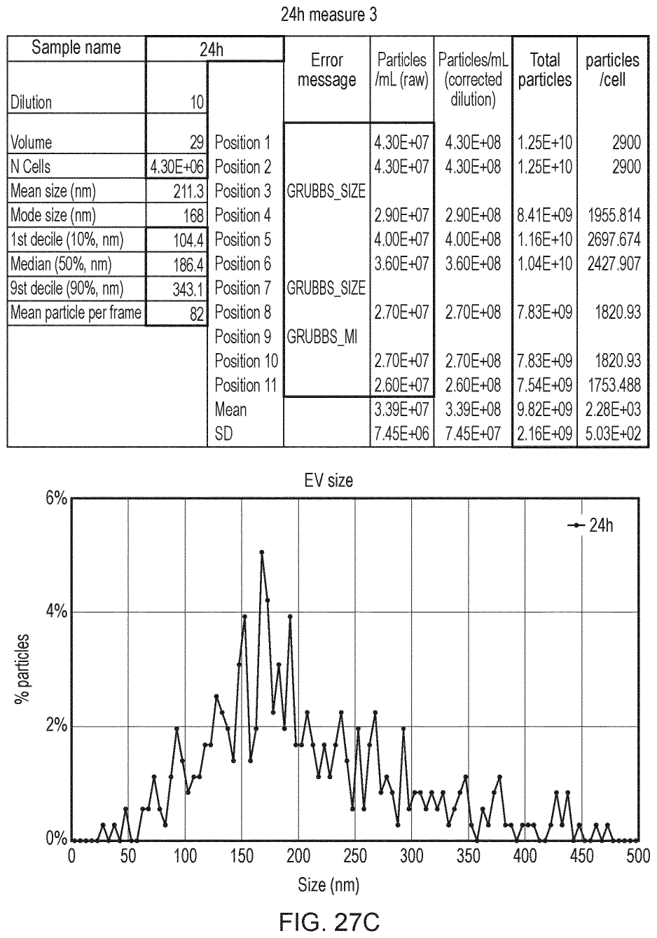

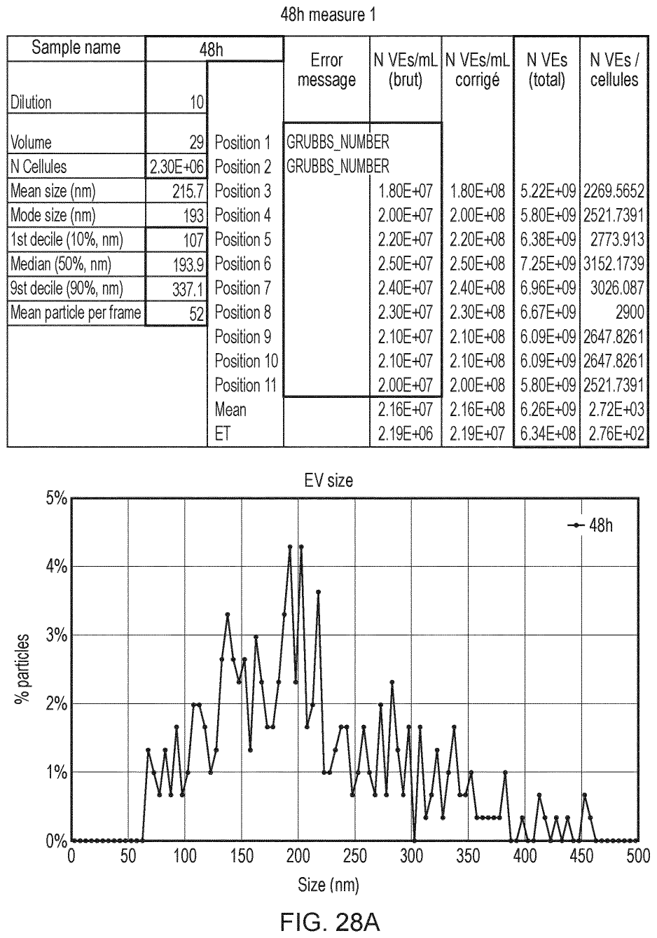

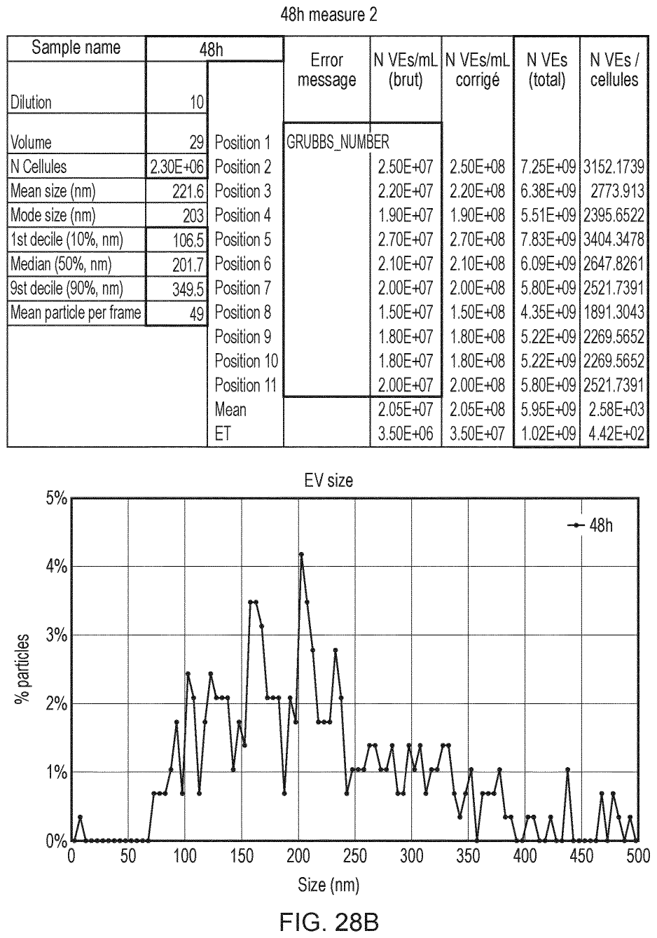

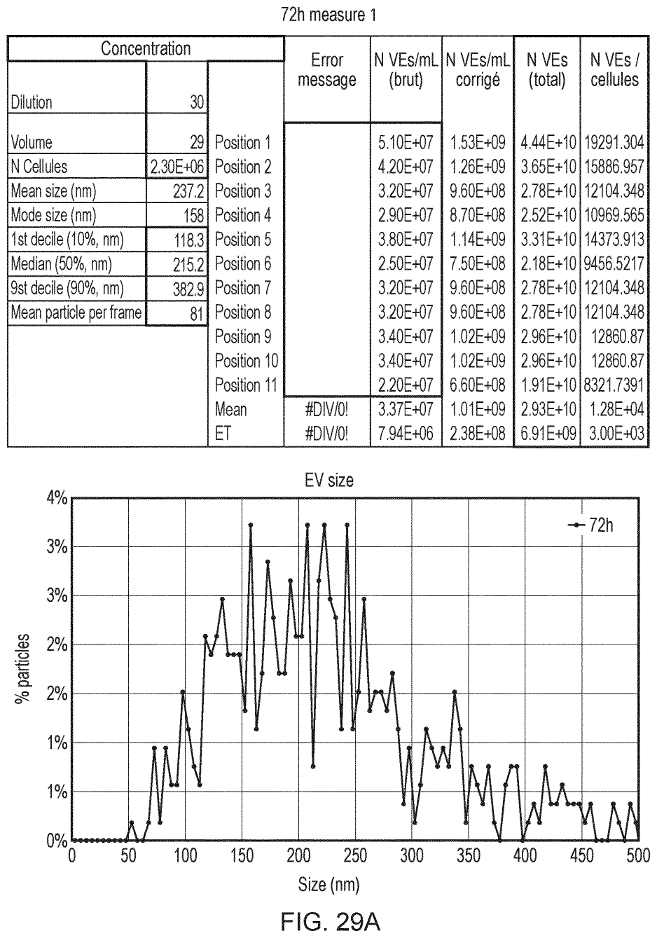

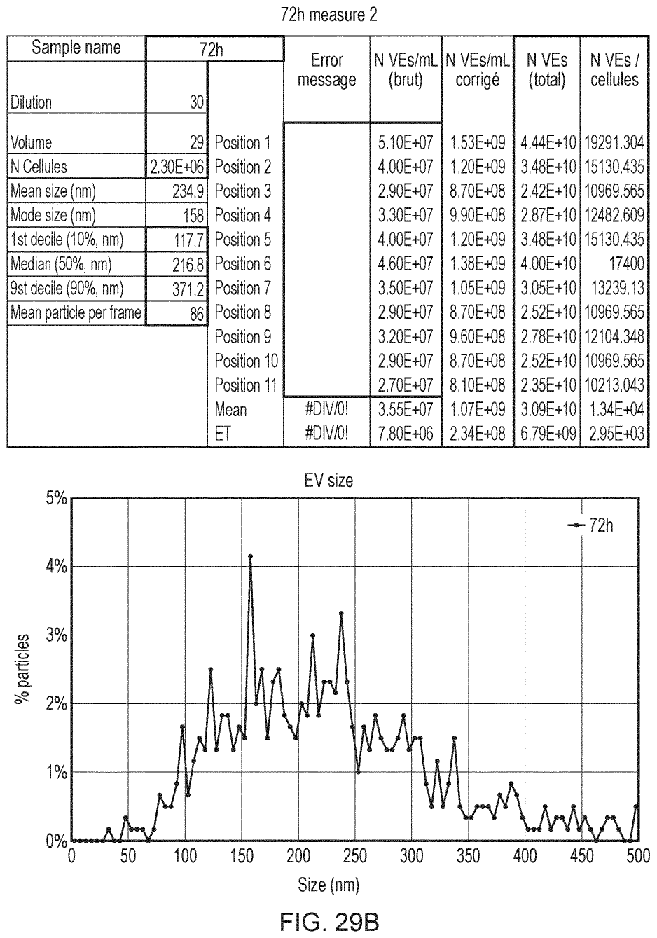

Table D summarises the results of the ZetaView analyses. Additional tabulated and graphical data can be seen in FIGS. 21 and 24-29. Scanning NTA was used to scan 11 positions, the mean of which is presented in Tables D1-D4. In some cases, the scanning NTA had an error message, which resulted in the position being excluded from the dataset as an anomaly.

TABLE D
NTA by ZetaView results (M = million) (the raw data used
to arrive at Table D can be seen in Tables D1, D2, D3 and D4).

| | | 24 h | 48 h | 72 h | Comparison |
|---|---|---|---|---|---|
| | T 0 | 4.3M | 2.3M | 2.3M | with hASC-EV |
| Cell count (% Viability) | (mean) | (98%) | (92%) | (96%) | values (3 D 4 h) |
| Mean size (nm) | 205 | 219 | 219 | 236 | 206 |
| Mode size (nm) | 163 | 159 | 198 | 158 | 186 |
| 1^{st} decile (10%, nm) | 94 | 105 | 107 | 118 | 105 |
| Median (50%, nm) | 175 | 189 | 198 | 216 | 187 |
| 9th decile (90%, nm) | 349 | 365 | 343 | 377 | 306 |
| Mean particle per frame | 21 | 82 | 51 | 84 | |
| Particles/mL (raw) | 8.2E+6 | 3.4E+07 | 2.1E+07 | 3.5E+07 | |
| Particles/mL (corrected dilution) | 50.8E+6 | 3.4E+08 | 2.1E+08 | 1.0E+09 | |
| Particles (total) | 1.5E+9 | 9.8E+09 | 6.1E+09 | 3.0E+10 | |
| Particles/cell | 354.3E+0 | 2.3E+03 | 2.7E+03 | 1.3E+04 | 32,000 |
| Corrected particle/cell | | 1,900 | | 10,000 | |
| Particle volume (nm³/mL) | 2.3E+14 | 1.9E+15 | 1.2E+15 | 7.2E+15 | |

### DISCUSSION

A low particle number was observed at t0, indicating a good production medium. Cell number reduced after 24 h starvation, then remained stable at around 50% until 72 h. Particle production increases after 48 h starvation, with a very high number and slightly higher size.

### Raw Data

TABLE D1
Raw data for the aliquots taken from the 24 h, 48 h and 72 h flasks at time zero (t0). These data represent
the samples taken at time zero from each of the 24 h, 48 h and 72 h flasks. Therefore, the mean in Table
D1 is that of all 6 samples, as all flasks are technically replicates of each other at time zero.

| t0 | 24 h | | 48 h | | 72 h | | Mean |
|---|---|---|---|---|---|---|---|
| Mean size (nm) | 201 | 238 | 199 | 204 | 205 | 181 | 205 |
| Mode size (nm) | 138 | 138 | 123 | 163 | 248 | 173 | 163 |
| 1^{st} decile (10%, nm) | 89 | 106 | 96 | 103 | 90 | 80 | 94 |
| Median (50%, nm) | 174 | 194 | 166 | 176 | 182 | 156 | 175 |
| 9th decile (90%, nm) | 354 | 420 | 347 | 343 | 321 | 307 | 349 |
| Mean particle per frame | 14 | 16 | 27 | 30 | 16 | 20 | 21 |
| Particles/mL (raw) | 5.9E+6 | 5.7E+6 | 10.9E+6 | 12.5E+6 | 7.7E+6 | 6.5E+6 | 8.2E+6 |
| Particles/mL (corrected | 59.4E+6 | 57.3E+6 | 54.3E+6 | 62.4E+6 | 38.6E+6 | 32.6E+6 | 50.8E+6 |
| dilution) | | | | | | | |
| Particles (total) | 1.8E+9 | 1.7E+9 | 1.6E+9 | 1.9E+9 | 1.2E+9 | 978.8E+6 | 1.5E+9 |
| Particles/cell | 414.7E+0 | 400.0E+0 | 378.9E+0 | 435.3E+0 | 269.0E+0 | 227.6E+0 | 354.3E+0 |
| Particle volume | | | | | | | 2.28E+14 |
| (nm³/mL) | | | | | | | |

TABLE D2
Raw data for the 24 h timepoint, n = 3 (SD = standard deviation).

| t 0 | Measure 1 | Measure 2 | Measure 3 | Mean | SD |
|---|---|---|---|---|---|
| Mean size (nm) | 227 | 217 | 211 | 219 | 8 |
| Mode size (nm) | 148 | 163 | 168 | 159 | 10 |
| 1^{st} decile (10%, nm) | 110 | 99 | 104 | 105 | 5 |
| Median (50%, nm) | 191 | 191 | 186 | 189 | 3 |
| 9th decile (90%, nm) | 403 | 349 | 343 | 365 | 33 |
| Mean particle per frame | 53 | 111 | 82 | 82 | 29 |
| Particles/ml (raw) | 22.0E+6 | 46.0E+6 | 33.9E+6 | 3.4E+07 | 120E+07 |
| Particles/mL (corrected | 220.0E+6 | 460.0E+6 | 338.8E+6 | 3.4E+08 | 120E+08 |
| dilution) | | | | | |
| Particles (total) | 6.4E+9 | 13.3E+9 | 9.8E+9 | 9.8E+09 | 3.5E+9 |
| Particles/cell | 1.5E+3 | 3.1E+3 | 2.3E+3 | 2.3E+03 | 809 |
| Particle volume (nm³/mL) | | | | 1.86E+15 | |

TABLE D3
Raw data for the 48 h timepoint, n = 3 (SD = standard deviation).

| t 0 | Measure 1 | Measure 2 | Mean | SD |
|---|---|---|---|---|
| Mean size (nm) | 216 | 222 | 219 | 4 |
| Mode size (nm) | 193 | 203 | 198 | 7 |
| 1^{st} decile (10%, nm) | 107 | 107 | 107 | 0 |
| Median (50%, nm) | 194 | 202 | 198 | 6 |
| 9th decile (90%, nm) | 337 | 350 | 343 | 9 |
| Mean particle per frame | 52 | 49 | 51 | 2 |
| Particles/mL (raw) | 21.6E+6 | 20.5E+6 | 2.1E+07 | 7.46E+05 |
| Particles/mL (corrected | 215.6E+6 | 205.0E+6 | 2.1E+08 | 7.46E+06 |
| dilution) | | | | |
| Particles (total) | 6.3E+9 | 5.9E+9 | 6.1E+09 | 2.16E+08 |
| Particles/cell | 2.7E+3 | 2.6E+3 | 2.7E+03 | 9.41E+01 |
| Particle volume (nm³/mL) | | | 1.15E+15 | |

TABLE D4
Raw data for the 72 h timepoint, n = 3 (SD = standard deviation).

| | Mea- | Mea- | | |
|---|---|---|---|---|
| t 0 | sure 1 | sure 2 | Mean | SD |
| Mean size (nm) | 237 | 235 | 236 | 2 |
| Mode size (nm) | 158 | 158 | 158 | — |
| 1^{st} decile (10%, nm) | 118 | 118 | 118 | 0 |
| Median (50%, nm) | 215 | 217 | 216 | 1 |
| 9th decile (90%, nm) | 383 | 371 | 377 | 8 |
| Mean particle per | 81 | 86 | 84 | 4 |
| frame | | | | |
| Particles/mL (raw) | 33.7E+6 | 35.5E+6 | 3.5E+07 | 1.29E+06 |
| Particles/mL | 1.0E+9 | 1.1E+9 | 1.0E+09 | 3.86E+07 |
| (corrected dilution) | | | | |
| Particles (total) | 29.3E+9 | 30.9E+9 | 3.0E+10 | 1.1E+9 |
| Particles/cell | 12.8E+3 | 13.4E+3 | 1.3E+04 | 486.3 |
| Particle volume | | | 7.16E+15 | |
| (nm³/mL) | | | | |

### Example 3—Characterisation of Extracellular Vesicles by ExoView

### Materials and Methods

ExoView® Tetraspanin Kits were used following manufacturer's instructions for all conditions described in Examples 1 and 2 (24 h, 48 h and 72 h).

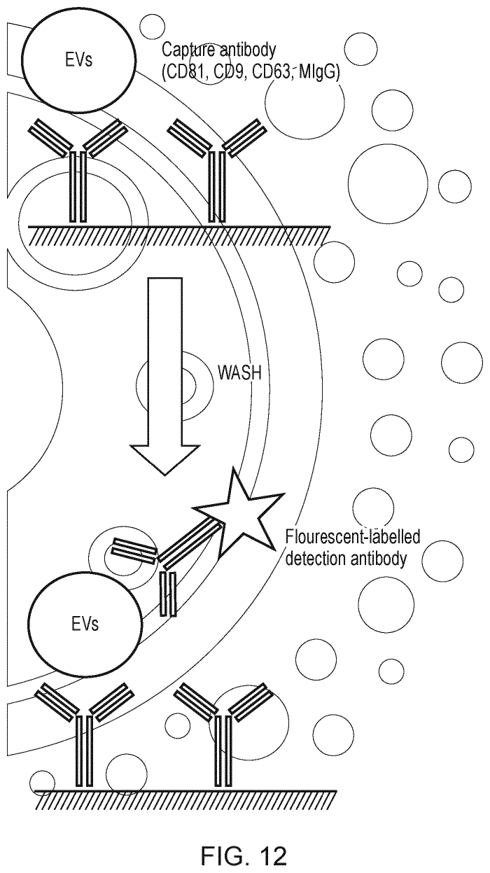

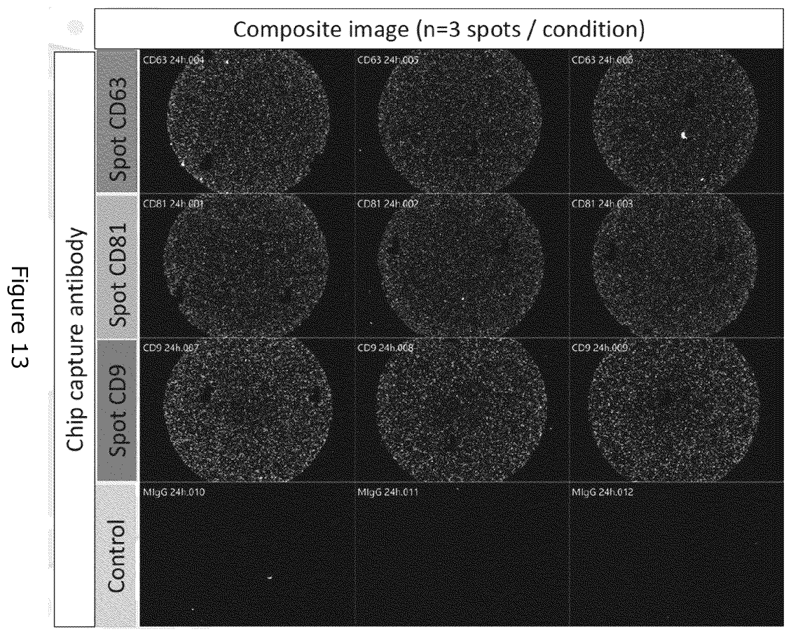

The main addressed research questions for the ExoView analyses are the measurement of particle numbers captured at each spot (based on CD81, CD63 and CD9 as surface markers, with murine IgG used as a negative control), and the level of co-expression of these 3 markers among EVs, with a recommended concentration of 10E+8 particles/mL. Larger batch sizes (i.e. the starting number of cells) can be used to obtain a recommended concentration, or incubation time can be extended (e.g. 72 h incubation increased the number of particles). A schematic of the ExoView analysis is shown in FIG. 12.

Composite images were obtained for three spots per condition, i.e. three replicates of spot CD63, three replicates of spot CD81, three replicates of spot CD9, and three spots of control (MIgG).

### Results

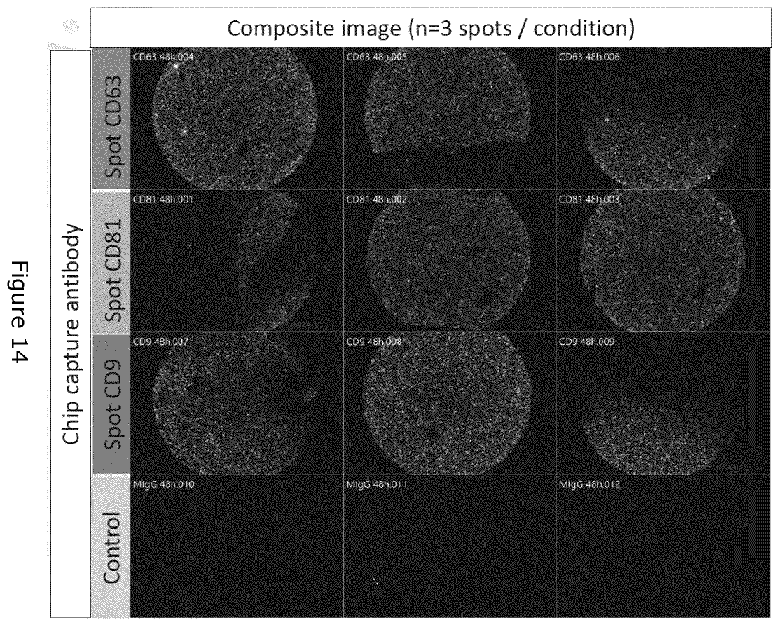

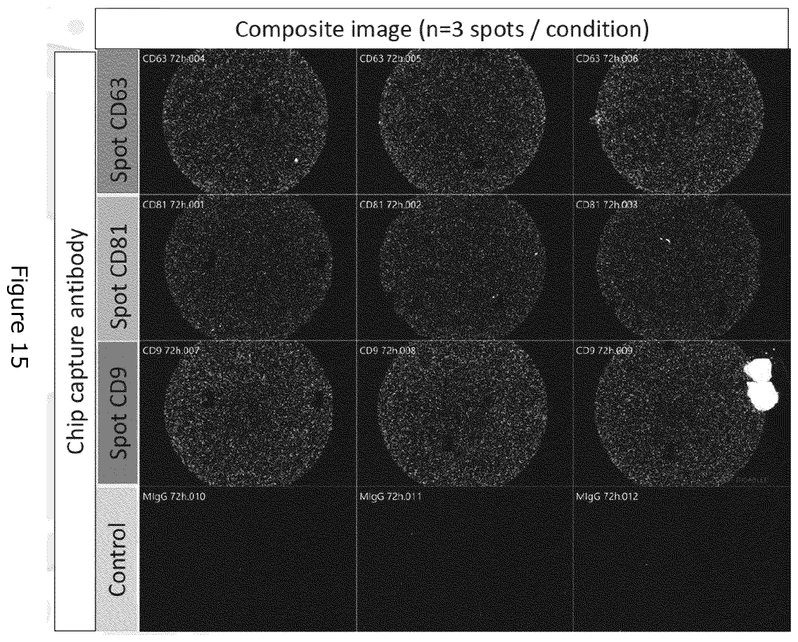

FIG. 13 demonstrates the spot montage obtained from the tetraspanin analysis by ExoView after 24 hours of incubation. Overall, the data show a good appearance, with similarity between the 3 replicates, no saturation, no artefact or strange patterns, and no signal on control spots. Control MIgG demonstrated no significant tetraspanin spots.

FIG. 14 demonstrates the spot montage obtained from the tetraspanin analysis by ExoView after 48 hours of incubation. The data demonstrate similarity between the 3 replicates, no saturation, no artefact or strange patterns, and no signal on control spots. One spot from CD81 (left panel, denoted in FIG. 14 as CD81 48 h.001, see FIG. 16, top panel) was disabled, and one spot from CD63 (middle panel, denoted in FIG. 14 as CD63 48 h.005), as no correct spot analysis could be obtained from these spots, which can be caused by excessive shadowing-such data is therefore excluded from the analysis. Control MIgG demonstrated no significant tetraspanin spots.

FIG. 15 demonstrates the spot montage obtained from the tetraspanin analysis by ExoView after 72 hours of incubation. The data demonstrate similarity between the 3 replicates, no saturation, no artefact or strange patterns, and no signal on control spots. One spot for CD9 (right panel, denoted in FIG. 15 as CD9 72 h.009, see FIG. 16, bottom panel) was disabled. Control MIgG demonstrated no significant tetraspanin spots.

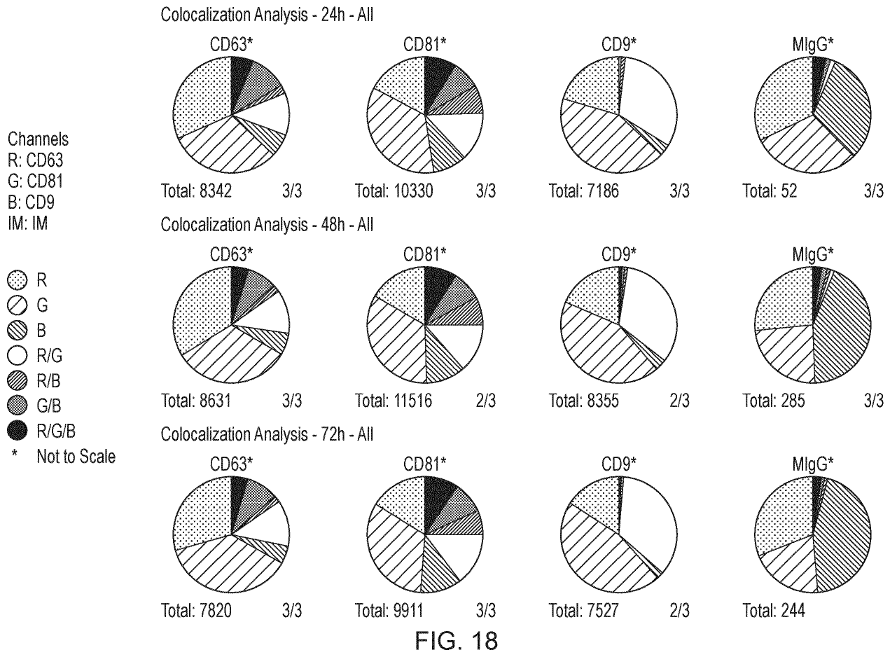

The spot montages are graphically represented in FIGS. 17 and 18, showing the number of positive particles at each spot for each fluorescent channel. A similar profile was observed for the 3 timepoints, with the highest number of positive particles observed for CD81, followed by CD63, with CD9 showing the lowest number of positive particles. A high presence of multi-staining (#33%) was observed. CD81 was over the linear range (>5000 particle/spot/channel), indicating that the number of positive particles to be a potential underestimation.

### Example 4—Comparison NTA and ExoView

### Materials and Methods

An arbitrary ratio (mean detected particles in all fluorescence channels per spot/total particle number incubated per ExoView chip, according to ZetaView) was prepared.

### Results

The results of the comparison NTA and ExoView are summarised in Table E.

TABLE E
ExoView/ZetaView ratio.

| ExoView/ZetaView ratio | CD63 | CD81 | CD9 |
|---|---|---|---|
| 24 h | 2.4E−03 | 3.0E−03 | 2.1E−03 |
| 48 h | 2.5E−03 | 3.3E−03 | 2.4E−03 |
| 72 h | 2.1E−03 | 2.8E−03 | 2.2E−03 |

A similar profile was observed for the three timepoints (24 h, 48 h and 72 h), with a ExoView/ZetaView ratio that was higher than hASC-EVs, indicating that the production of EVs from TAF MSCs achieves a higher purity than production of EVs from hASCs (human adipose stem cells).

All timepoints present EV with a comparable high purity. High CD81 tetraspanin was observed, with a significant presence of the CD63 and CD9 tetraspanins also. Particle size increased for the 72 h timepoint, as well as the quantity of EVs increasing. The total particle number obtained from the 72 h aliquot was 3×10¹⁰ particle number (i.e. number of EVs) in total (see Table D), which is enough for further characterisation of the EVs.

### Discussion

An increase in particle size can occur as they go into a senescent state. Alternatively, the increase in particle size may be due to serum starvation. If the size increases too much, it may be indicative of undesirous apoptotic vesicles forming. However, the level of increase in size is not sufficient to be attributable to the formation of apoptotic vesicles.

### Example 5—MACSPlex and Immunomodulation Assay

### Materials and Methods

Skin specific TAF MSCs were used to generate these data. Sample 1, denoted as «72UC-EV», corresponds to 26 mL of the 72 h secretome purified by ultracentrifugation, and the pellet containing EVs being resuspended in 1 mL PBS. Sample 2, denoted as «<72-WS», corresponds to the 72 h whole secretome (i.e. not purified).

Both samples were dosed for protein content (Bradford Assay) and then frozen at −80° C. Following the thawing of both samples 3 days later, they were submitted to the following characterisation:
1. Nanoparticle Tracking Analysis (NTA) measurement: Size+Concentration by ZetaView;
2. MACSPlex: multiplexed marker analysis by flow cytometry (following manufacturer's protocol); and
3. Immunomodulation assay: action on the NFκB pathway.

### Results—NTA Measurement

The NTA results are summarised in Table F. NTA was performed after one freeze-thaw cycle, which may have resulted in the lower concentration of 72-WS than was previously observed (1.0×10⁹ particles/mL). 72UC-EV retained 74% particles compared to the 72-WS, which is a good concentration yield. There was no significant size different between pelleted EVs and the whole secretome.

TABLE F
NTA by ZetaView (SD = standard deviation).

| | 72UC-EV | | 72-WS | |
|---|---|---|---|---|
| 72UC-EV | Mean | SD | Mean | SD |
| Mean size (nm) | 210 | 5 | 200 | 6 |
| Mode size (nm) | 151 | 18 | 132 | 8 |
| 1^{st} decile (10%, nm) | 106 | 2 | 103 | 6 |
| Median (50%, nm) | 183 | 9 | 180 | 2 |
| 9^{th} decile (90%, nm) | 343 | 9 | 311 | 13 |
| Mean particle per frame | 68 | 10 | 69 | 3 |
| Particles/mL (raw) | 2.8E+07 | 4.4E+06 | 2.9E+07 | 1.4E+06 |
| Particles/mL (corrected | 1.1E+10 | 1.7E+09 | 5.8E+08 | 2.8E+07 |
| dilution) | | | | |

### Results—MACSPlex and Bradford Assay

A Bradford Assay was used to assess the protein concentration of the two samples, as summarised in Table G.

TABLE G
Bradford Assay results.

| Sample | Protein concentration | Particle/μg protein ratio |
|---|---|---|
| 72UC-EV | 24.4 μg/mL | 4.9E+8 |
| 72-WS | 22.5 μg/mL | 2.4E+7 |

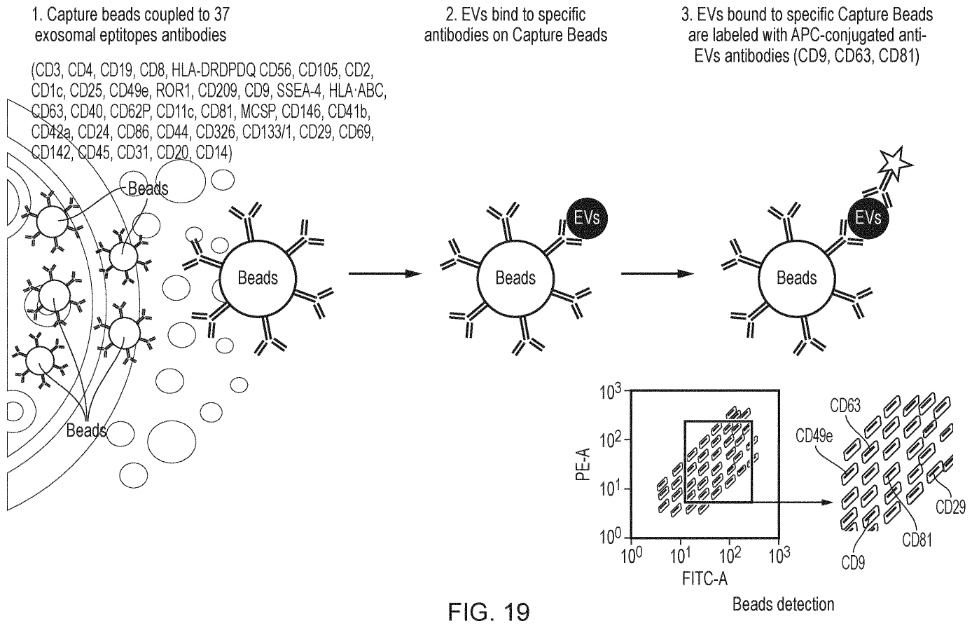

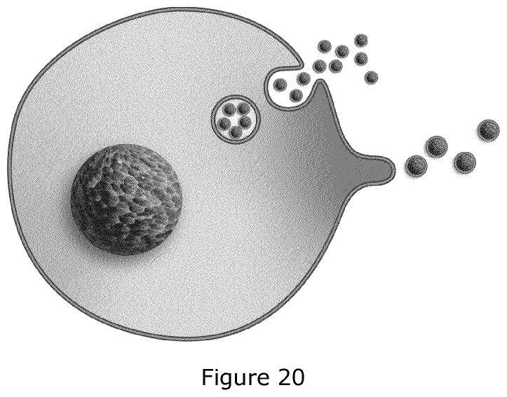

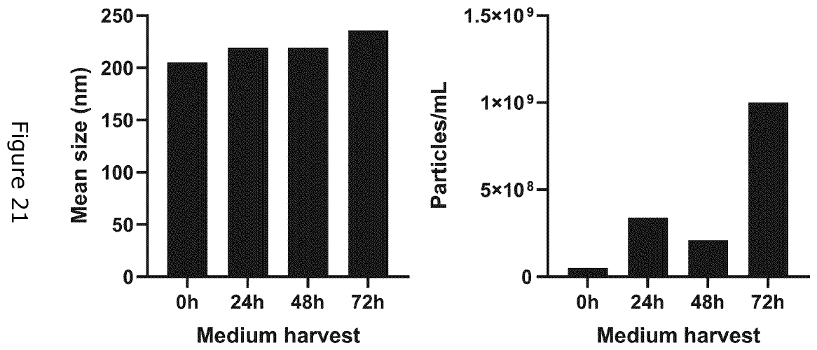

The MACSPlex protocol used capture beads coupled to 37 exosomal epitope antibodies, as diagrammatically represented in FIG. 19. The epitopes were CD3, CD4, CD19, CD8, HLA-DRDPDQ, CD56, CD105, CD2, CD1c, CD25, CD49e, ROR1, CD209, CD9, SSEA-4, HLA-ABC, CD63, CD40, CD62P, CD11c, CD81, MCSP, CD146, CD41b, CD42a, CD24, CD86, CD44, CD326, CD133/1, CD29, CD69, CD142, CD45, CD31, CD20, and CD14. EVs bound to specific capture beads were subsequently labelled with APC-conjugated to anti-EV antibodies (CD9, CD63, CD81), and detected using flow cytometry.

The MASCPlex results are summarised in FIG. 23. Fluorescence was normalised based on a control sample that contained no EVs (i.e. a blank control). Normalised fluorescence >2 indicates a significant presence of a particular marker. Fluorescence level is associated to the quantity of positive particles.

Exosome markers (CD9, CD24, CD29, CD63, CD81) are highly present, and ultracentrifugation further enhanced their concentration. MSC markers (CD105 and CD146) were also positive in the 72UC-EV sample. Ultracentrifugation purification did not remove any positive markers from the EVs of the whole secretome. Immune cell markers were mostly negative for EVs.

### Results—Immunomodulation Assay

THP-1 dual cells were used to generate these data, which are derived from THP-1 monocytic cell line with the stable integration of two inducible reporter constructs. THP-1 dual allows the study of the NFκB pathway by monitoring the activity of SEAP, the reporter protein. The THP-1 dual cells were treated with LPS as a positive control, dexamethasone as a negative control, EVs, or a combination of the aforementioned (see Table H). Optimal EV concentration for these tests was 1E9 EVs/mL. The maximum volume of supernatant was added to each well, as the EV concentration for the samples was too low to reach 1E9 EVs/mL (see Table I). Despite not always having an optimal EV concentration, the EV amounts used showed effects in the immunomodulation assay. Accordingly, a further improved effect would be expected upon using an optimal amount of EVs.

TABLE H
Key for the conditions tested in immunomodulation assay.

| 1 | 2 | 3 | 4 |
|---|---|---|---|
| Control (LPS) | Dexamethasone + | | 72UC-EV + |
| | LPS | | LPS |
| Control (LPS) | Dexamethasone + | 72-WS + | 72UC-EV + |
| | LPS | LPS | LPS |
| Control (LPS) | Dexamethasone + | 72-WS + | 72UC-EV + |
| | LPS | LPS | LPS |
| Control (nothing | Dexamethasone only | 72-WS only | 72UC-EV only |
| added) | | | |
| Control (nothing | Dexamethasone only | 72-WS only | 72UC-EV only |
| added) | | | |
| Control (nothing | Dexamethasone only | | 72UC-EV only |
| added) | | | |

TABLE I
Particle and protein concentrations for the two samples.

| Final concentration | Particle/mL | Protein μg/mL |
|---|---|---|
| 72-WS | 1.9 10⁸ | 7.5 |
| 72UC-EV | 1.0 10⁹ | 2.0 |

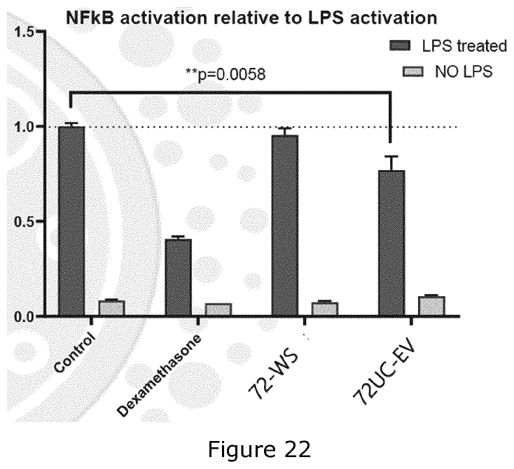

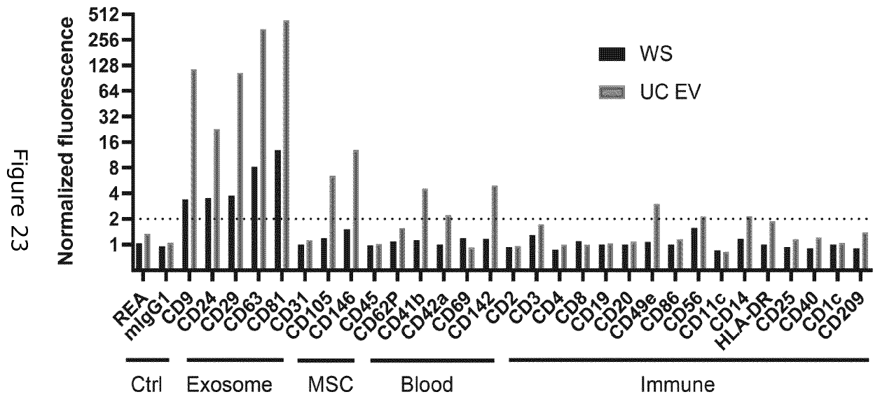

The results of the immunomodulation assay are graphically represented in FIG. 22, in which all values are normalised to LPS activation of the NFκB pathway. EVs and supernatant inherently do not trigger inflammation. EVs at optimal concentration reduce inflammation. 72-WS does not reduce inflammation, which indicates that the EV fraction is responsible for the immunomodulation, and not the free proteins that are also present in the whole secretome.

### Discussion

Given the anti-inflammatory properties observed for the isolated EVs, it follows that such EVs are likely to be effective in treating wounds. Such uses can be readily demonstrated in assays known to the skilled person, such as a scratch test for testing tissue regeneration.

### Items

Item 1. A method for obtaining extracellular vesicles from term amniotic fluid mesenchymal stem cells (TAF MSCs), comprising:
providing term amniotic fluid (TAF);
removing particulate material from the TAF to obtain purified TAF cells;
performing adherence selection on the purified TAF cells to obtain TAF adherence cells;
passaging the TAF adherence cells to obtain a population of cells comprising TAF MSCs;
propagating the TAF MSCs; and
obtaining extracellular vesicles from the TAF MSCs.
   Item 2. The method according to item 1, wherein the method further comprises selecting the TAF MSCs from the population of TAF cells that express at least one surface marker associated with skin TAF MSCs, lung TAF MSCs, kidney TAF MSCs, or neural TAF MSCs;
optionally wherein the selecting step:
   a. enriches the population of TAF MSCs to comprise at least 10%, 20%, 30%, 40%, 50%, 60%, 70%, 80%, 90%, 95%, 96%, 97%, 98%, 99% or 100% skin TAF MSCs, lung TAF MSCs, kidney TAF MSCs, or neural TAF MSCs;
   b. is performed after the passaging step and/or before the propagating step.
      Item 3. The method according to item 1 or 2, wherein the method further comprises preconditioning the TAF MSCs, optionally wherein the preconditioning is by serum-starvation, inflammation and/or hypoxia.
      Item 4. The method according to any preceding item, wherein the extracellular vesicles are exosome vesicles and/or microvesicles.
      Item 5. The method according to any preceding item, wherein the extracellular vesicles are:
a. less than 1000 nm in diameter;
b. greater than 20 nm in diameter (to exclude apoptotic vesicles that are 1000-5000 nm); or
c. 20-1000 nm in diameter;
when measured using Nanoparticle Tracking Analysis (NTA)
   Item 6. The method according to any preceding item, wherein the extracellular vesicles express at least one surface marker associated with skin TAF MSCs, lung TAF MSCs, kidney TAF MSCs, or neural TAF MSCs.
   Item 7. The method according to any preceding item, wherein the at least one surface marker is selected from the group consisting of:
a. MME (CD10);
b. MME (CD10), TNFSF18, PCDH19, NCAM2, TNFSF4, CD248, DDR2, HTR2B, PCDH18, SULF1, ADGRA2, DCSTAMP, PDGFRA, UNC5B, SCUBE3, CEMIP, BDKRB1, FLT1, BDKRB2, FAP, CASP1, and SRPX2;
c. TBC1 domain family member 3K, allograft inflammatory factor 1 like, cadherin related family member 1, sodium/potassium transporting ATPase interacting 4, ATP binding cassette subfamily B member 1, plasmalemma vesicle associated protein, mesothelin, L1 cell adhesion molecule, hepatitis A virus cellular receptor 1, mal, T cell differentiation protein 2 (gene/pseudogene), SLAM family member 7, double C2 domain beta, endothelial cell adhesion molecule, gamma-aminobutyric acid type A receptor beta1 subunit, cadherin 16, immunoglobulin superfamily member 3, desmocollin 3, regulator of hemoglobinization and erythroid cell expansion, potassium voltage-gated channel interacting protein 1, CD70 molecule, GDNF family receptor alpha 1, crumbs cell polarity complex component 3, claudin 1, novel transcript sodium voltage-gated channel alpha subunit 5, fibroblast growth factor receptor 4, potassium two pore domain channel subfamily K member 3, dysferlin, ephrin A1, potassium inwardly rectifying channel subfamily J member 16, membrane associated ring-CH-type finger 1, synaptotagmin like 1, calsyntenin 2, integrin subunit beta 4, vesicle associated membrane protein 8, G protein-coupled receptor class C group 5 member C, CD24 molecule, cadherin EGF LAG seven-pass G-type receptor 2, cadherin 8, glutamate receptor interacting protein 1, dematin actin binding protein, F11 receptor, cell adhesion molecule 1, cadherin 6, coagulation factor II thrombin receptor like 2, LY6/PLAUR domain containing 1, solute carrier family 6 member 6, desmoglein 2, adhesion G protein-coupled receptor G1, cholecystokinin A receptor, oxytocin receptor, integrin subunit alpha 3, adhesion molecule with Ig like domain 2, cadherin EGF LAG seven-pass G-type receptor 1, and EPH receptor B2;
d. PCDH19, DDR1, MME (CD10), IFITM10, BGN, NOTCH3, SULF1, TNFSF18, BDKRB1, FLT1, PDGFRA, TNFSF4, UNC5B, FAP, CASP1, CD248, DDR2, PCDH18, LRRC38, and CRLF1;
e. HAVCR1, CD24, CLDN6, ABCB1, SHISA9, CRB3, AC118754.1, ITGB6, CDH1, LSR, EPCAM, AJAP1, ANO9, CLDN7, EFNA1, MAL2, F11R, L1CAM, GFRA1, IGSF3, TNF, MMP7, FOLR1, TGFA, C3, TNFSF10, PDGFB and WWC1; or
f. HAVCR1, ACKR3, OSCAR, C3, SIRPB1, SLC6A6, CCKAR, TNFSF10, CLSTN2, TENM2, SFRP1, PIK3IP1, SCNN1D, CLDN11, ALDH3B1, and ITGB4.
   Item 8. Isolated extracellular vesicles, wherein:
a. the isolated extracellular vesicles are produced by the method according to any of items 1-7;
b. the purity is a ratio of based on an ExoView/ZetaView ratio;
c. the isolated extracellular vesicles express at least one surface marker associated with skin TAF MSCs, lung TAF MSCs, kidney TAF MSCs, or neural TAF MSCs; and/or
d. the isolated extracellular vesicles express at least one surface marker selected from the group consisting of:
   i. MME (CD10);
   ii. MME (CD10), TNFSF18, PCDH19, NCAM2, TNFSF4, CD248, DDR2, HTR2B, PCDH18, SULF1, ADGRA2, DCSTAMP, PDGFRA, UNC5B, SCUBE3, CEMIP, BDKRB1, FLT1, BDKRB2, FAP, CASP1, and SRPX2;
   iii. TBC1 domain family member 3K, allograft inflammatory factor 1 like, cadherin related family member 1, sodium/potassium transporting ATPase interacting 4, ATP binding cassette subfamily B member 1, plasmalemma vesicle associated protein, mesothelin, L1 cell adhesion molecule, hepatitis A virus cellular receptor 1, mal, T cell differentiation protein 2 (gene/pseudogene), SLAM family member 7, double C2 domain beta, endothelial cell adhesion molecule, gamma-aminobutyric acid type A receptor beta1 subunit, cadherin 16, immunoglobulin superfamily member 3, desmocollin 3, regulator of hemoglobinization and erythroid cell expansion, potassium voltage-gated channel interacting protein 1, CD70 molecule, GDNF family receptor alpha 1, crumbs cell polarity complex component 3, claudin 1, novel transcript sodium voltage-gated channel alpha subunit 5, fibroblast growth factor receptor 4, potassium two pore domain channel subfamily K member 3, dysferlin, ephrin A1, potassium inwardly rectifying channel subfamily J member 16, membrane associated ring-CH-type finger 1, synaptotagmin like 1, calsyntenin 2, integrin subunit beta 4, vesicle associated membrane protein 8, G protein-coupled receptor class C group 5 member C, CD24 molecule, cadherin EGF LAG seven-pass G-type receptor 2, cadherin 8, glutamate receptor interacting protein 1, dematin actin binding protein, F11 receptor, cell adhesion molecule 1, cadherin 6, coagulation factor II thrombin receptor like 2, LY6/PLAUR domain containing 1, solute carrier family 6 member 6, desmoglein 2, adhesion G protein-coupled receptor G1, cholecystokinin A receptor, oxytocin receptor, integrin subunit alpha 3, adhesion molecule with Ig like domain 2, cadherin EGF LAG seven-pass G-type receptor 1, and EPH receptor B2;
   iv. PCDH19, DDR1, MME (CD10), IFITM10, BGN, NOTCH3, SULF1, TNFSF18, BDKRB1, FLT1, PDGFRA, TNFSF4, UNC5B, FAP, CASP1, CD248, DDR2, PCDH18, LRRC38, and CRLF1;
   v. HAVCR1, CD24, CLDN6, ABCB1, SHISA9, CRB3, AC118754.1, ITGB6, CDH1, LSR, EPCAM, AJAP1, ANO9, CLDN7, EFNA1, MAL2, F11R, L1CAM, GFRA1, IGSF3, TNF, MMP7, FOLR1, TGFA, C3, TNFSF10, PDGFB and WWC1; or
   vi. HAVCR1, ACKR3, OSCAR, C3, SIRPB1, SLC6A6, CCKAR, TNFSF10, CLSTN2, TENM2, SFRP1, PIK3IP1, SCNN1D, CLDN11, ALDH3B1, and ITGB4.
      Item 9. A decellularised composition comprising the isolated extracellular vesicles according to item 8,
and optionally comprising at least one pharmaceutically acceptable carrier, excipient or further component such as therapeutic and/or prophylactic ingredient.
   Item 10. Isolated extracellular vesicles according to item 8, or the decellularised composition according to item 8, for use in medicine.
   Item 11. Isolated extracellular vesicles according to item 8, or the decellularised composition according to item 9, for use in preventing and/or treating:
a. a skin condition (such as chronic skin ulcers, also referred to as chronic cutaneous ulcers, diabetic skin ulcers, bedsores);
b. fibrotic diseases (such as scar formation);
c. cancer or tumours, multiple sclerosis, amyotrophic lateral sclerosis, cardiovascular diseases (e.g. stroke, acute and chronic heart failure, atherosclerosis), diabetes, arthritis (e.g. rheumatoid arthritis and osteoarthritis), osteonecrosis, lumbar intervertebral disc degeneration, bowel disease (e.g. Crohn's disease), kidney and liver chronic disease, sepsis, spinal cord contusions, critical limb ischemia, neurodegenerative diseases, atherosclerosis;
d. skin, renal, liver, and neural injuries;
e. an adverse immune response, such as inflammation; and/or
f. transplantation/GVHD.
   Item 12. Isolated extracellular vesicles according to item 8, or the decellularised composition according to item 9, for use in wound healing, optionally wherein the wound is of the skin, lung, kidney, neural, liver, heart (and heart valves), trachea, body parts (such as limbs/digits), pancreas, intestine, colon and combinations thereof.
   Item 13. The isolated extracellular vesicles or the decellularised composition for use according to any of items 10-12, wherein the isolated extracellular vesicles or the decellularised composition are/is administered by means selected from intravenously, topically, intramuscularly, intradermally, and intraarterially.
   Item 14. The isolated extracellular vesicles or the decellularised composition for use according to any of items 10-12, wherein the isolated extracellular vesicles or the decellularised composition are/is administered in combination with a further agent.
   Item 15. A non-therapeutic use of the isolated extracellular vesicles according to item 8, or the decellularised composition according to item 9, in antiaging.
   Item 16. A device comprising and/or embedded with the isolated extracellular vesicles according to item 8, or the decellularised composition according to item 9, optionally wherein the device is selected from the group consisting of a hydrogel, a dressing, a bandage, a suture, and a plaster.

### REFERENCES

The listing or discussion of an apparently prior-published document in this specification should not necessarily be taken as an acknowledgement that the document is part of the state of the art or is common general knowledge. The references disclosed, to the extent that they provide exemplary procedural or other details supplementary to those set forth herein, are specifically incorporated herein by reference.
Casado-Días, Quesada-Gómez and Dorado, Extracellular vesicles derived from mesenchymal stem cells in regenerative medicine: Applications in skin wound healing, Frontiers in bioengineering and biotechnology, March 2020, Vol 8, Article 146.
WO 2021/076042 A1, Amniotics AB.
WO 2021/076043 A1, Amniotics AB.

1. A method for obtaining extracellular vesicles from term amniotic fluid mesenchymal stem cells (TAF MSCs), the method comprising:
providing term amniotic fluid (TAF);
removing particulate material from the TAF to obtain purified TAF cells;
performing adherence selection on the purified TAF cells to obtain TAF adherence cells;
passaging the TAF adherence cells to obtain a population of cells comprising TAF MSCs;
propagating the TAF MSCs; and
obtaining extracellular vesicles from the TAF MSCs.

2-10. (canceled)

11. The method according to claim 1, wherein the method further comprises selecting the TAF MSCs from the population of TAF cells that express at least one surface marker associated with skin TAF MSCs, lung TAF MSCs, kidney TAF MSCs, or neural TAF MSCs.

12. The method according to claim 11, wherein the selecting step:
a. enriches the population of TAF MSCs to comprise at least 10%, 20%, 30%, 40%, 50%, 60%, 70%, 80%, 90%, 95%, 96%, 97%, 98%, 99% or 100% skin TAF MSCs, lung TAF MSCs, kidney TAF MSCs, or neural TAF MSCs; or
b. is performed after the passaging step or before the propagating step.

13. The method according to claim 1, wherein:
the extracellular vesicles express at least one surface marker associated with skin TAF MSCs, lung TAF MSCs, kidney TAF MSCs, or neural TAF MSCs; or
the at least one surface marker is selected from the group consisting of:
a. MME (CD10);
b. MME (CD10), TNFSF18, PCDH19, NCAM2, TNFSF4, CD248, DDR2, HTR2B, PCDH18, SULF1, ADGRA2, DCSTAMP, PDGFRA, UNC5B, SCUBE3, CEMIP, BDKRB1, FLT1, BDKRB2, FAP, CASP1, and SRPX2;
c. TBC1 domain family member 3K, allograft inflammatory factor 1 like, cadherin related family member 1, sodium/potassium transporting ATPase interacting 4, ATP binding cassette subfamily B member 1, plasmalemma vesicle associated protein, mesothelin, L1 cell adhesion molecule, hepatitis A virus cellular receptor 1, mal, T cell differentiation protein 2 (gene/pseudogene), SLAM family member 7, double C2 domain beta, endothelial cell adhesion molecule, gamma-aminobutyric acid type A receptor beta1 subunit, cadherin 16, immunoglobulin superfamily member 3, desmocollin 3, regulator of hemoglobinization and erythroid cell expansion, potassium voltage-gated channel interacting protein 1, CD70 molecule, GDNF family receptor alpha 1, crumbs cell polarity complex component 3, claudin 1, novel transcript sodium voltage-gated channel alpha subunit 5, fibroblast growth factor receptor 4, potassium two pore domain channel subfamily K member 3, dysferlin, ephrin A1, potassium inwardly rectifying channel subfamily J member 16, membrane associated ring-CH-type finger 1, synaptotagmin like 1, calsyntenin 2, integrin subunit beta 4, vesicle associated membrane protein 8, G protein-coupled receptor class C group 5 member C, CD24 molecule, cadherin EGF LAG seven-pass G-type receptor 2, cadherin 8, glutamate receptor interacting protein 1, dematin actin binding protein, F11 receptor, cell adhesion molecule 1, cadherin 6, coagulation factor II thrombin receptor like 2, LY6/PLAUR domain containing 1, solute carrier family 6 member 6, desmoglein 2, adhesion G protein-coupled receptor G1, cholecystokinin A receptor, oxytocin receptor, integrin subunit alpha 3, adhesion molecule with Ig like domain 2, cadherin EGF LAG seven-pass G-type receptor 1, and EPH receptor B2;
d. PCDH19, DDR1, MME (CD10), IFITM10, BGN, NOTCH3, SULF1, TNFSF18, BDKRB1, FLT1, PDGFRA, TNFSF4, UNC5B, FAP, CASP1, CD248, DDR2, PCDH18, LRRC38, and CRLF1;
c. HAVCR, CD24, CLDN6, ABCB1, SHISA9, CRB3, AC118754.1, ITGB6, CDH1, LSR, EPCAM, AJAP1, ANO9, CLDN7, EFNA1, MAL2, F1IR, L1CAM, GFRA1, IGSF3, TNF, MMP7, FOLR1, TGFA, C3, TNFSF10, PDGFB and WWC1; and
f. HAVCR1, ACKR3, OSCAR, C3, SIRPB1, SLC6A6, CCKAR, TNFSF10, CLSTN2, TENM2, SFRP1, PIK3IP1, SCNN1D, CLDN11, ALDH3B1, and ITGB4.

14. Isolated extracellular vesicles, wherein:
a. the purity is a ratio of based on an Exo View/Zeta View ratio;
b. the isolated extracellular vesicles express at least one surface marker associated with skin TAF MSCs, lung TAF MSCs, kidney TAF MSCs, or neural TAF MSCs; or
c. the isolated extracellular vesicles express at least one surface marker selected from the group consisting of:
i. MME (CD10);
ii. MME (CD10), TNFSF18, PCDH19, NCAM2, TNFSF4, CD248, DDR2, HTR2B, PCDH18, SULF1, ADGRA2, DCSTAMP, PDGFRA, UNC5B, SCUBE3, CEMIP, BDKRB1, FLT1, BDKRB2, FAP, CASP1, and SRPX2;
iii. TBC1 domain family member 3K, allograft inflammatory factor 1 like, cadherin related family member 1, sodium/potassium transporting ATPase interacting 4, ATP binding cassette subfamily B member 1, plasmalemma vesicle associated protein, mesothelin, L1 cell adhesion molecule, hepatitis A virus cellular receptor 1, mal, T cell differentiation protein 2 (gene/pseudogene), SLAM family member 7, double C2 domain beta, endothelial cell adhesion molecule, gamma-aminobutyric acid type A receptor beta1 subunit, cadherin 16, immunoglobulin superfamily member 3, desmocollin 3, regulator of hemoglobinization and erythroid cell expansion, potassium voltage-gated channel interacting protein 1, CD70 molecule, GDNF family receptor alpha 1, crumbs cell polarity complex component 3, claudin 1, novel transcript sodium voltage-gated channel alpha subunit 5, fibroblast growth factor receptor 4, potassium two pore domain channel subfamily K member 3, dysferlin, ephrin A1, potassium inwardly rectifying channel subfamily J member 16, membrane associated ring-CH-type finger 1, synaptotagmin like 1, calsyntenin 2, integrin subunit beta 4, vesicle associated membrane protein 8, G protein-coupled receptor class C group 5 member C, CD24 molecule, cadherin EGF LAG seven-pass G-type receptor 2, cadherin 8, glutamate receptor interacting protein 1, dematin actin binding protein, F11 receptor, cell adhesion molecule 1, cadherin 6, coagulation factor II thrombin receptor like 2, LY6/PLAUR domain containing 1, solute carrier family 6 member 6, desmoglein 2, adhesion G protein-coupled receptor G1, cholecystokinin A receptor, oxytocin receptor, integrin subunit alpha 3, adhesion molecule with Ig like domain 2, cadherin EGF LAG seven-pass G-type receptor 1, and EPH receptor B2;
iv. PCDH19, DDR1, MME (CD10), IFITM10, BGN, NOTCH3, SULF1, TNFSF18, BDKRB1, FLT1, PDGFRA, TNFSF4, UNC5B, FAP, CASP1, CD248, DDR2, PCDH18, LRRC38, and CRLF1;
v. HAVCR1, CD24, CLDN6, ABCB1, SHISA9, CRB3, AC118754.1, ITGB6, CDH1, LSR, EPCAM, AJAP1, ANO9, CLDN7, EFNA1, MAL2, F11R, L1CAM, GFRA1, IGSF3, TNF, MMP7, FOLR1, TGFA, C3, TNFSF10, PDGFB and WWC1; and
vi. HAVCR1, ACKR3, OSCAR, C3, SIRPB1, SLC6A6, CCKAR, TNFSF10, CLSTN2, TENM2, SFRP1, PIK3IP1, SCNN1D, CLDN11, ALDH3B1, and ITGB4.

15. A decellularised composition comprising the isolated extracellular vesicles according to claim 14.

16. The decellularised composition of claim 15, further comprising at least one pharmaceutically acceptable carrier, excipient or further component.

17. The decellularised composition of claim 16, wherein the further component comprises a therapeutic or prophylactic ingredient.

18. A method of administering a skin wound healing therapy, comprising administering the isolated extracellular vesicles according to claim 14 to a subject in need thereof.

19. A method of preventing or treating a disease or condition in a subject, comprising:
administering the isolated extracellular vesicles according to claim 14 to a subject having a disease or condition, wherein the disease or condition is:
a skin condition, a fibrotic disease, a cancer, a tumour, multiple sclerosis, amyotrophic lateral sclerosis, a cardiovascular disease, diabetes, arthritis, osteonecrosis, lumbar intervertebral disc degeneration, bowel disease, kidney chronic disease, liver chronic disease, sepsis, spinal cord contusions, critical limb ischemia, neurodegenerative diseases, atherosclerosis, a skin injury, a renal injury, a liver injury, a neural injury, an adverse immune response, inflammation, transplantation, or graft versus host disease (GVHD).

20. The method of claim 19, wherein the skin condition comprises chronic skin ulcers, diabetic skin ulcers, or bedsores.

21. The method of claim 19, wherein the fibrotic disease comprises scar formation.

22. The method of claim 19, wherein the cardiovascular disease comprises stroke, acute heart failure, chronic heart failure, or atherosclerosis.

23. The method of claim 19, wherein the arthritis comprises rheumatoid arthritis or osteoarthritis.

24. The method of claim 19, wherein the bowel disease comprises Crohn's disease.

25. A method of healing a wound in a subject, comprising administering the isolated extracellular vesicles according to claim 14.

26. The method of claim 25, wherein the wound is of the skin, lung, kidney, neural, liver, heart, heart valves, trachea, body part, limb, digit, pancreas, intestine, colon or a combination thereof.

27. A method of promoting antiaging in a subject, comprising administering the isolated extracellular vesicles according to claim 14 to a subject in need thereof.

28. A device comprising or embedded with the isolated extracellular vesicles according to claim 14.

29. The device of claim 28, wherein the device is selected from the group consisting of a hydrogel, a dressing, a bandage, a suture, and a plaster.
